(19) United States
(12) Patent Application Publication
(10) Pub. No.: US 2025/0188417 A1
(43) Pub. Date: Jun. 12, 2025

(54) CARDIOMYOCYTES AND COMPOSITIONS AND METHODS FOR PRODUCING THE SAME

(71) Applicants: President and Fellows of Harvard College, Cambridge, MA (US); THE CHILDREN'S MEDICAL CENTER CORPORATION, Boston, MA (US)

(72) Inventors: Richard T. Lee, Cambridge, MA (US); Jessica Garbern, Brookline, MA (US)

(21) Appl. No.: 18/286,405

(22) PCT Filed: Mar. 29, 2022
(86) PCT No.: PCT/US2022/022402

§ 371 Date: Oct. 11, 2023

Related U.S. Application Data
(60) Provisional application No. 63/173,489, filed on Apr. 11, 2021.

(51) Int. Cl. C12N 5/077; A61K 35/34

(52) U.S. Cl. CPC C12N 5/0657; A61K 35/34; C12N 2501/999; C12N 2513/00

(57) ABSTRACT

Disclosed herein are methods for generating mature cardiomyocytes and compositions including mature cardiomyocytes. Also disclosed herein are methods for enhancing maturation of quiescent cardiomyocytes and compositions including mature quiescent cardiomyocytes.

### CROSS-REFERENCE TO RELATED APPLICATIONS

This application claims the benefit of U.S. Provisional Application No. 63/173,489, filed on Apr. 11, 2021. The entire teachings of the above application are incorporated herein by reference.

### BACKGROUND OF THE INVENTION

Current differentiation protocols to produce cardiomyocytes from human induced pluripotent stem cells (iPSCs) are capable of generating highly pure cardiomyocyte populations as determined by expression of cardiac troponin T. However, these cardiomyocytes remain immature, more closely resembling the fetal state, with a lower maximum contractile force, slower upstroke velocity, and immature mitochondrial function compared with adult cardiomyocytes. Immaturity of iPSC-derived cardiomyocytes may be a significant barrier to clinical translation of cardiomyocyte cell therapies for heart disease because the immature cardiomyocytes display automaticity or pacemaker-like activity, which results in potentially life-threatening ventricular arrhythmias when delivered to adult animal models, as well as having a less organized sarcomere structure preventing adequate contractile force. During development, cardiomyocytes undergo a shift from a proliferative state in the fetus to a more mature but quiescent state after birth. There exists a need for improved methods for leading cardiomyocytes to a quiescent state and thus enhancing cardiomyocyte maturation during differentiation.

### SUMMARY OF THE INVENTION

There is a need for methods or protocols for the generation of mature cardiomyocytes for use in cell therapy and screening, among other uses. In some embodiments, by leading cardiomyocytes to a quiescent state, maturation of the cardiomyocytes may be enhanced.

Disclosed herein are methods of producing a mature cardiomyocyte from an immature cardiomyocyte. The methods comprise contacting the immature cardiomyocyte with at least one cardiomyocyte maturation factor in culture, e.g., in two-dimensional or three-dimensional culture, such as pulsed or steady state culture.

Also disclosed herein are methods of inducing quiescence of a cardiomyocyte. The methods comprise contacting a senescent cardiomyocyte with a cardiomyocyte maturation factor in culture, e.g., two-dimensional or three-dimensional culture, such as pulsed or steady state culture, thereby inducing the senescent cardiomyocyte to transition into a quiescent cardiomyocyte.

In some embodiments, the at least one cardiomyocyte maturation factor comprises a p53 upregulator. In some embodiments, the at least one cardiomyocyte maturation factor comprises an mTOR inhibitor. In some embodiments, the at least one cardiomyocyte maturation factor comprises a p53 upregulator and/or an mTOR inhibitor. In some embodiments, the at least one cardiomyocyte maturation factor comprises a senolytic, a MDM2 inhibitor, and/or an mTOR inhibitor. In some embodiments, the at least one cardiomyocyte maturation factor is selected from the group consisting of a senolytic, a MDM2 inhibitor, an mTOR inhibitor, and combinations thereof. In some embodiments, the at least one cardiomyocyte maturation factor is selected from the group consisting of nutlin-3a, quercetin, Torin1, and combinations thereof. In some embodiments, the at least one cardiomyocyte maturation factor is selected from the group consisting of nutlin-3a, quercetin, and combinations thereof. In some embodiments, the at least one cardiomyocyte maturation factor comprises nutlin-3a and/or quercetin. In some embodiments, the at least one cardiomyocyte maturation factor comprises nutlin-3a, quercetin, and/or Torin1. In some embodiments, the at least one cardiomyocyte maturation factor is contacted with the immature cardiomyocyte via pulse treatment or via continuous treatment.

In some embodiments, the immature cardiomyocyte is derived from an iPS cell, an ES cell, a T cell, or a fibroblast. In some embodiments, the immature cardiomyocyte resembles a fetal cardiomyocyte.

In some embodiments, the immature cardiomyocyte is contacted with the at least one cardiomyocyte maturation factor after the immature cardiomyocyte begins beating. In some embodiments, the immature cardiomyocyte is contacted with the at least one cardiomyocyte maturation factor 1 to 3 days after the immature cardiomyocyte begins beating.

In some embodiments, the mature cardiomyocyte exhibits increased expression of one or more genes of maturation as compared to an immature cardiomyocyte. In some embodiments, the one or more genes of maturation are selected from the group consisting of: TNNI3, TNNT2, MYH6, MYH7, NPPB, HCN4, CACNA1c, SERCA2a, PPARGC1, KCNJ2, REST, RyR, and SCN5a. In some embodiments, the mature cardiomyocyte exhibits increased expression of one or more sarcomeric proteins as compared to an immature cardiomyocyte. In some embodiments, the one or more sarcomeric proteins are selected from the group consisting of: TNNT2, TNNI3, MYH6, and MYH7. In some embodiments, the mature cardiomyocyte exhibits increased expression of one or more ion channel genes as compared to an immature cardiomyocyte. In some embodiments, the one or more ion channel genes are selected from the group consisting of: KCNJ2, HCN4, SCN5a, RYR2, CACNA1c, and SERCA2a. In some embodiments, the mature cardiomyocyte exhibits increased expression of REST and/or GATA4 as compared to an immature cardiomyocyte.

In some embodiments, the mature cardiomyocyte exhibits a decreased beating rate as compared to an immature cardiomyocyte. In some embodiments, the mature cardiomyocyte exhibits a resting membrane potential of ≤−70 mV, a spontaneous beating rate of <40 beats per minute, and/or an upstroke velocity of >200 V/sec. In some embodiments, the mature cardiomyocyte is an electrically, contractility, and/or metabolically mature cardiomyocyte.

Also disclosed herein are non-naturally occurring cardiomyocytes produced by any of the methods disclosed herein.

In some embodiments, the non-naturally occurring cardiomyocyte exhibits increased expression of one or more genes of maturation as compared to an immature cardiomyocyte. In some embodiments, the one or more genes of maturation are selected from the group consisting of: TNNI3, TNNT2, MYH6, MYH7, NPPB, HCN4, CACNA1c, SERCA2a, PPARGC1, KCNJ2, REST, RyR, and SCN5a. In some embodiments, the non-naturally occurring cardiomyocyte exhibits increased expression of one or more sarcomeric proteins as compared to an immature cardiomyocyte. In some embodiments, the one or more sarcomeric proteins are selected from the group consisting of: TNNT2, TNNI3, MYH6, and MYH7. In some embodiments, the non-naturally occurring cardiomyocyte exhibits increased expression of one or more ion channel genes as compared to an immature cardiomyocyte. In some embodiments, the one or more ion channel genes are selected from the group consisting of: KCNJ2, HCN4, SCN5a, RYR2, CACNA1c, and SERCA2a. In some embodiments, the non-naturally occurring cardiomyocyte exhibits increased expression of one or more of TNNT2, TNNI3, KCNJ2, and p53.

In some embodiments, the non-naturally occurring cardiomyocyte exhibits a decreased beating rate as compared to an immature cardiomyocyte. In some embodiments, the non-naturally occurring cardiomyocyte exhibits a resting membrane potential of ≤−70 mV, a spontaneous beating rate of <40 beats per minute, and/or an upstroke velocity of >200 V/sec. In some embodiments, the non-naturally occurring cardiomyocyte is an electrically, contractility, and/or metabolically mature cardiomyocyte.

Disclosed herein are methods of treatment comprising administering to a subject in need thereof a composition comprising a non-naturally occurring cardiomyocyte described herein. Also disclosed herein are methods of treatment comprising administering to a subject in need thereof a pharmaceutical composition produced using an isolated population of one or more non-naturally occurring cardiomyocytes described herein. Also disclosed herein are uses of a composition in the manufacture of a medicament for treatment of a heart condition, wherein the treatment comprises administration of the medicament to a subject in need thereof, wherein the composition comprises at least one non-naturally occurring cardiomyocyte described herein.

In some embodiments, the subject has, or is at risk of developing, a ventricular arrhythmia, decreased systolic heart function, chronic heart failure, congenital heart disease, or other heart disease.

Also disclosed herein are methods of producing a mature cardiomyocyte from an immature cardiomyocyte. The methods comprise contacting the immature cardiomyocyte with at least one cardiomyocyte maturation factor selected from the group consisting of nutlin-3a, quercetin, Torin1, and combinations thereof.

In some embodiments, the cardiomyocyte maturation factor is selected from the group consisting of nutlin-3a, quercetin, and combinations thereof. In some embodiments, the mature cardiomyocytes are produced in three-dimensional culture. In some embodiments, the mature cardiomyocytes are produced in two-dimensional culture. In some embodiments, the immature cardiomyocytes are contacted with the at least one cardiomyocyte maturation factor via a pulse treatment. In some embodiments, the immature cardiomyocytes are contacted with the at least one cardiomyocyte maturation factor via a continuous treatment.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™ McKusick-Nathans Institute of Genetic Medicine, Johns Hopkins University (Baltimore, MD) and National Center for Biotechnology Information, National Library of Medicine (Bethesda, MD), as of May 1, 2010, World Wide Web URL: ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 provides a schematic of a proposal to create electrically mature iPSC-CMs in suspension culture.

FIGS. 2A-2H demonstrate nutlin-3a increases percentage of TNNT2+ iPSC-CMs (FIG. 2A), mean fluorescence intensity (MFI) of TNNT2 (FIG. 2B), % of TNNI3+ (FIG. 2C), mean fluorescence intensity of TNNI3 (FIG. 2D), percentage of TNNT2+ iPSC-CMs expressing Kir2.1 (FIG. 2E), mean fluorescence intensity (MFI) of Kir2.1 (FIG. 2F), % of p53+ TNNT2+ iPSC-CMs (FIG. 2G), and mean fluorescence intensity of TNNI3 and p53 by flow cytometry (FIG. 2F).

FIGS. 3A-3D demonstrate quercetin increases % TNNT2+ iPSC-CMs expressing Kir2.1 (FIG. 3A), mean fluorescence intensity (MFI) of Kir2.1 (FIG. 3B), % of p53+ TNNT2+ iPSC-CMs (FIG. 3C), and MFI of p53 by flow cytometry (FIG. 3D).

FIGS. 4A-4B demonstrate quercetin decreases the percentage of live cells but increases the percentage of cardiomyocytes. FIG. 4A shows percentage of live cells. FIG. 4B shows percentage of TNNT2+ cells out of live cells. Quantification by flow cytometry. **p<0.01, ***p<0.001, ****p<0.0001 by one-way ANOVA.

FIG. 5 shows quercetin increases expression of PPARGC1a. ****p<0.0001 by Kruskal-Wallis test.

FIGS. 6A-6E demonstrate upregulation of p53 with nutlin-3a enhances TNNI3 expression and has a synergistic effect with Torin1. FIG. 6A provides a schematic showing proposed mechanism of cardiomyocyte maturation. T3, triiodothyronine. FIG. 6B shows cell cycle analysis showing percentage of TNNT2+ BJRiPS-CMs in G0, G1, or S/G2/M phases after treatment with vehicle (DMSO), nutlin-3a (10 mol/L) for 24 hours, Torin1 (200 nmol/L) for 7 days or simultaneous treatment with nutlin-3a (10 mol/L for first 24 hours of Torin1 treatment) with Torin1 (200 nmol/L) for 7 days starting ≈2 days after onset of beating. n=3 per group, *P<0.05, ***P<0.001, ****P<0.0001 by 2-way ANOVA with Tukey multiple comparisons test. FIG. 6C provides representative Western blot of MDM2, p53, TNNI3, and β-tubulin from BJRiPS-derived cardiomyocyte lysates treated with control (DMSO), nutlin-3a Å˜24 hours (10 μmol/L), Torin1 Å˜7 days (200 nmol/L), or Torin1 Å˜7 days (200 nmol/L)+nutlin-3a (10 μmol/L) for 24 hours beginning ≈2 days after onset of beating (nutlin-3a was only administered during the first 24 hours of Torin1 treatment). Cells harvested on final day of treatment, BJRiPS-CMs. FIG. 6D shows densitometry of p53 bands by Western analysis, **P<0.01 by 2-way ANOVA with Tukey multiple comparisons test, n=3 per condition. FIG. 6E shows densitometry of TNNI3 bands by Western analysis, *P<0.05, **P<0.01 by 2-way ANOVA with Tukey multiple comparisons test, n=3 per condition, BJRIPS-CMs. DMSO indicates dimethylsulfoxide; mTOR, mechanistic target of rapamycin; and TNNT2+ BJRiPS-CMs, troponin T2, cardiac type-positive BJ fibroblast-derived RNA-induced pluripotent stem cell-derived cardiomyocytes.

FIGS. 7A-7B demonstrate Torin1+/−Nutlin-3a increases TNNT2 expression (FIG. 7A) and increases mitochondrial membrane polarization (FIG. 7B) in 2D culture. This suggests more mature mitochondria in the cardiomyocytes. FACS N=3/group, MitoTracker Red CMXRos flow cytometry, 1-way ANOVA with Tukey's multiple comparisons test.

FIG. 8 shows Nutlin-3a treatment in 2D culture increases mean fluorescence intensity of superoxide signal. Reactive oxygen species may regulate cardiomyocyte maturation. FACS, n=3/group.

FIGS. 9A-9B demonstrate Torin1+Nutlin-3a treatment increases TNNT2 purity (FIG. 9A) and TNNT2 expression (FIG. 9B) in iPSC-cardiomyocytes differentiated in 3D suspension culture. Cells pulse-treated for 1 hour every 24 hours×2 days in 3D suspension culture starting on day 10 of cardiomyocyte differentiation. Data from flow cytometry, n=3 per group.

FIGS. 10A-10I demonstrates Torin1 treatment increases cellular quiescence of induced pluripotent stem cell (iPSC)-derived cardiomyocytes (CMs). FIG. 10A provides a schematic of a differentiation protocol, with Torin1 treatment performed for 7 days starting ˜2 days after onset of beating, unless otherwise noted. FIG. 10B provides a western analysis of phospho-S6 and phospho-Akt at baseline and 30 minutes, and 2, 4, 10, 24, and 48 hours after a single treatment of iPSC-derived CMs with Torin1 (200 nM), BJRiPS-CMs. FIG. 10C shows cell counts per well of a 12 well plate during differentiation. Torin1 treatment started on day 9, approximately 2 days after onset of CM beating, BJRiPS-CMs, n=3 per group per time point. FIG. 10D shows percentage of TNNT2+ CMs out of live cell population by flow cytometry, Gibco iPS-CMs, n=3 per condition, n.s. by one-way ANOVA. FIG. 10E shows qPCR of selected quiescence markers (TP53, RB1, RBL2 (p130), CDKN1a (p21), CDKN1b (p27), CDKN2a (p16) and HES1) and proliferation markers (MKI67, CCNA1, CCNB1, CCNC1, CCND1, CDK3, and E2F11 iPSC-derived cardiomyocytes after treatment with Torin1 (10 nM, 50 nM, or 200 nM) or vehicle control (0.02% DMSO) for 7 days starting ˜2 days after onset of beating. n=3 per condition, *p<0.05, ****p<0.001 by multiple t-tests with Holm-Sidak method to compare to control for each gene, BJRiPS-CMs. FIG. 10F provides representative flow cytometry plots of iPSC-derived CMs stained with Hoechst 33342 and Pyronin Y to distinguish between G0, G1, and S/G2/M phases in control, 10 nM Torin1-treated, or 200 nM Torin1-treated cells, Gibco iPS-CMs. FIG. 10G shows percentage of TNNT2+ cardiomyocytes in G₀, G₁ or S/G₂/M phases after Torin1-treatment (200 nM) for 1 week starting ˜2 days after onset of beating. n=3 per condition, ***p<0.001, ****p<0.0001 by two-way ANOVA with Tukey's multiple comparisons test, Gibco iPS-CMs. FIG. 10H shows percentage of TNNT2+ CMs in G₀, G₁ or S/G₂/M phases in control CMs after 0.02% DMSO-treatment for 1 week starting ˜2 days after onset of beating followed by 10% FBS or no serum control. n=3 per group, **p<0.01 by two-way ANOVA with Sidak's multiple comparisons test, Gibco iPSC-CMs. FIG. 10I shows percentage of TNNT2+ CMs in G₀, G₁ or S/G₂/M phases after Torin1-treatment (200 nM) for 1 week starting ˜2 days after onset of beating followed by 10% FBS or no serum control. n=3 per group, **p<0.01 by two-way ANOVA with Sidak's multiple comparisons test, Gibco iPSC-CMs. DMSO, dimethylsulfoxide; IWP-4, inhibitor of Wnt production-4; RI, ROCK (Rho-associated, coiled coil containing protein kinase) inhibitor (Y-27632); RPMI, Roswell Park Memorial Institute 1640 medium.

FIGS. 11A-11I demonstrate Torin1-treatment increases expression of sarcomere genes and enhances contractility of iPSC-derived cardiomyocytes. FIG. 11A provides qPCR of selected sarcomere genes (MYH6, MYH7, TNNT2, TNNI3) iPSC-derived cardiomyocytes after treatment with Torin1 (10 nM, 50 nM, or 200 nM) or vehicle control (0.02% DMSO) for 7 days starting ˜2 days after onset of beating. n=3 per condition, *p<0.05, ****p<0.001 by two-way ANOVA with Tukey's multiple comparisons test, BJRiPS-CMs. FIG. 11B provides representative of western blot analysis of TNNT2 and TNNI3 after Torin1 treatment for 7 days starting ˜2 days after onset of beating, β-tubulin depicted as loading control, Gibco iPS-CMs. FIG. 11C shows mean fluorescence intensity of TNNT2-Alexa Fluor 647 of TNNT2+ cells. n=3 per condition, **p<0.01, ****p<0.0001 by one-way ANOVA with Tukey's multiple comparisons test, Gibco iPS-CMs. FIG. 11D provides representative image of MTF in diastole and systole, with schematic of MTF on side view in diastole and systole. FIG. 11E provides representative force versus time plot of muscular thin film (MTF) seeded with iPSC-derived cardiomyocytes treated with Torin1 (200 nM) or vehicle for 7 days starting ˜2 days after onset of beating, BJRiPS-CMs. FIG. 11F shows relative maximum systolic force (normalized to control) as quantified using muscular thin films. n=8-9 per group, **p<0.01 by Kruskal-Wallis test, combined from 3 batches of BJRiPS-CMs (2 batches) and Gibco iPS-CMs (1 batch). FIG. 11G provides representative immunostained images showing cardiomyocytes treated with or without Torin1 (200 nM×7 days), blue=DAPI, green=alpha-actinin, yellow=F-actin, magenta=TNNT2, BJRiPS-CMs, scale bar=10 μm. FIG. 11H shows sarcomere length of control (n=13 cells) and Torin1-treated (n=17 cells) BJRiPS-CMs, n.s by unpaired t-test. FIG. 11I shows mean fluorescence intensity of TNNT2-Alexa Fluor 647 of TNNT2+ cells after treatment with DMSO×7 days (control), DMSO×7 days followed by 10% fetal bovine serum (FBS)×2 days, Torin1 (200 nM)×7 days, or Torin1 (200 nM)×7 days followed by 10% FBS×2 days. n=3 per condition, *p<0.01, **p<0.0001 by one-way ANOVA with Sidak's multiple comparisons test, UCSD-CMs. DMSO, dimethylsulfoxide; TBP, TATA-binding protein.

FIGS. 12A-12F demonstrate Torin1 increases oxygen consumption rate and mitochondrial polarization of iPSC-derived cardiomyocytes. FIG. 12A provides a profile of average oxygen consumption rate normalized to baseline versus time as evaluated by the Seahorse Mito Stress Test. Open circles=control (n=70 wells), closed squares=Torin1 (69 wells, 200 nM×7 days; BJRiPS-CMs replated into Seahorse plate for 24-48 hours prior to assay), data from two independent experiments combined. FIG. 12B shows oxygen consumption rate normalized to baseline of control (open bars, n=70 wells) versus closed bars (open bars, n=69 wells) for maximum OCR, respiratory reserve capacity, and non-mitochondrial OCR, *p<0.05, ***p<0.001 by two-way ANOVA with Sidak's multiple comparisons test, BJRiPS-CMs. FIG. 12C shows mean fluorescence intensity of Mitotracker Green FM of BJRiPS-CMs after treatment with Torin1 (200 nM) or vehicle control (0.02% DMSO) for 7 days starting ˜2 days after onset of beating. *p<0.05 by unpaired t-test, n=3 per group, BJRiPS cell line. FIG. 12D shows mitochondrial (ND1) to nuclear (B2M) DNA ratio of BJRiPS-CMs after treatment with Torin1 (200 nM) or vehicle control (0.02% DMSO) for 7 days starting ˜2 days after onset of beating. *p<0.05 by unpaired t-test, n=3 per group, BJRiPS cell line. FIG. 12E shows MitoProbe JC-1 relative ratio of red to green fluorescence in BJRiPS-CMs after treatment with Torin1 (200 nM) or vehicle control (0.02% DMSO) for 7 days starting ˜2 days after onset of beating, assay run on final day of Torin1 treatment. n=6 per group, **p<0.01 by Kruskal-Wallis test. FIG. 12F provides qPCR of selected genes associated with fatty acid metabolism (PPARGC1a (PGC1α), CD36, SLC27A1 (FATP1), SLC27A6 (FATP6), and LPIN1) or glucose metabolism (GLUT1, GLUT4, PFK, and PYGM) of BJRiPS-CMs after treatment with Torin1 (10 nM, 50 nM, or 200 nM) or vehicle control (0.02% DMSO) for 7 days starting ˜2 days after onset of beating. *p<0.05, **p<0.01, ***p<0.001, ****p<0.0001 by two-way ANOVA with Tukey's multiple comparison's test, n=3 per group. DMSO, dimethylsulfoxide; FCCP, 2-[2-[4-(trifluoromethoxy)phenyl]hydrazinylidene]-propanedinitrile.

FIGS. 13A-13J demonstrate Torin1-treatment increases expression selected ion channels and increases peak rise time and downstroke velocity of the action potential profile. FIG. 13A provides qPCR of selected ion channels (KCNJ2, CACNA1c, RY2, ATP2a2, SCN5a, HCN4) of iPSC-derived cardiomyocytes after treatment with Torin1 (10 nM or 200 nM) or vehicle control (0.02% DMSO) for 7 days starting ˜2 days after onset of beating. n=3 per group, *p<0.05, ****p<0.001 by two-way ANOVA with Tukey's multiple comparisons test, BJRiPS line. FIG. 13B provides flow cytometry analysis of mean fluorescence intensity of Kir2.1 (encoded by KCNJ2) after Torin1 treatment for 7 days starting ˜2 days after onset of beating. n=3 per group, *p<0.01 by unpaired t-test, BJRiPS line. FIG. 13C shows spontaneous beating rate of cardiomyocytes with or without Torin1-treatment, n=6-10 wells per condition, *p<0.05, ***p<0.001 by one-way ANOVA with Tukey's multiple comparisons test. FIG. 13D provides representative images showing cell segmentation performed automatically by the CyteSeer software. Blue=Hoechst stain, Green=FluoVolt. FIG. 13E shows representative action potential profile depicting a Torin1-treated cardiomyocyte with a more prolonged plateau phase versus control. CTD25 (25% duration of the calcium transient, or duration at 25% decline from maximum amplitude), CTD75 (75% duration of the calcium transient, or duration at 75% decline from maximum amplitude), T75-25 (time for voltage to decay from 75% to 25% of maximum). FIG. 13F shows peak rise time (msec), ****p<0.0001, UCSD-CMs. FIG. 13G shows downstroke velocity (msec), ****p<0.0001, UCSD-CMs. FIG. 13H shows CTD25 time (msec), n.s., UCSD-CMs. FIG. 13I shows CTD75 time (msec), *p<0.05, UCSD-CMs. FIG. 13J shows T75-25 time (msec), ***p<0.001, UCSD-CMs. For Fluovolt data, control n=531 cells, Torin1 n=315 cells, analysis by unpaired t-test.

FIGS. 14A-14D demonstrate Torin1 increases p53 expression and effects are inhibited by pifithrin-α. FIG. 14A provides representative western blot of p53, phospho-53, p21 (CDKN1a), GATA4, NKX2.5, and β-tubulin from Gibco iPS-CM lysates treated with 0 (DMSO), 10, 50, or 200 nM Torin1 for 7 days starting ˜2 days after onset of beating. Cells harvested on final day of treatment. FIG. 14B provides cell cycle analysis showing percentage of TNNT2+ BJRiPS-CMs in G₀, G₁ or S/G₂/M phases after treatment with vehicle (DMSO, dimethylsulfoxide), pifithrin-α (10 μM) for 1 week, Torin1 (200 nM) for 7 days or simultaneous treatment with pifithrin-α (10 μM) and Torin1 (200 nM) for 7 days starting ˜2 days after onset of beating. n=3 per group, *p<0.05, **p<0.01, ***p<0.001, ****p<0.0001 by two-way ANOVA with Tukey's multiple comparisons test. FIG. 14C provides representative western blot of p53, TNNI3, p21, and β-tubulin from BJRiPS-derived cardiomyocyte lysates treated with control (DMSO), Torin1 (200 nM), pifithrin-α (10 PM) or Torin1 (200 nM)+pifithrin-α (10 μM) for 7 days beginning ˜2 days after onset of beating. Cells harvested on final day of treatment. FIG. 14D shows densitometry of TNNI3 bands by western analysis, n=3 per group, **p<0.01 by two-way ANOVA with Tukey's multiple comparisons test, BJRiPS-CMs.

FIGS. 15A-15H demonstrate NanoString gene expression analysis from PanCancer Pathways Panel comparing cells treated with or without Torin1 followed by treatment with or without 10% fetal bovine serum (FBS), Gibco iPS-CMs. FIG. 15A shows unsupervised hierarchical clustering of Cell Cycle pathway genes. FIG. 15B shows unsupervised hierarchical clustering of Metabolism of Proteins pathway genes. FIGS. 15C-15E provide volcano plots showing differential gene expression analysis of Cell Cycle pathway genes (FIG. 15C, Control+FBS versus Control; FIG. 15D, Control versus Torin1; FIG. 15E, Torin1+FBS versus Torin1). FIGS. 15F-15H provide volcano plots showing differential gene expression analysis of Metabolism of Proteins pathway genes (FIG. 15F, Control+FBS versus Control; FIG. 15G, Control versus Torin1; FIG. 15H, Torin1+FBS versus Torin1). QC, quality control.

FIGS. 16A-16C provide an evaluation of proliferation in non-dissociated cells. Cardiomyocytes were differentiated in 12-well plates then fixed and stained in the original plates to minimize potential for selection of cells that survive dissociation. FIG. 16A provides representative images of cells stained with Ki67/TNNT2/DAPI or phospho-H3/TNNT2/DAPI. Scale bar=100 μm. Blue=DAPI, yellow=Ki67, green=pH3, magenta=TNNT2. FIG. 16B provides pie charts depicting the percentage of total DAPI/TNNT+ cardiomyocytes that are Ki67+ or Ki67− in control (n=1088 cells) versus Torin1-treated cells (n=837 cells). ****p<0.0001 by chi-squared analysis. FIG. 16C provides pie charts depicting the percentage of total DAPI/TNNT+ cardiomyocytes that are pH3+ or pH3− in control (n=962 cells) versus Torin1-treated cells (n=779 cells). DMSO, dimethylsulfoxide.

FIGS. 17A-17I provide qPCR analysis of selected genes (FIG. 17A, TNNT2; FIG. 17B, TNNI3; FIG. 17C, PPARGC1a; FIG. 17D, RYR2; FIG. 17E, KCNJ2; FIG. 17F, CACNA1c; FIG. 17G, TP53; FIG. 17H, CDKN1a (p21); FIG. 17I, GATA4) compared to benchmarking samples of commercially available human fetal and adult heart RNA. N=6-12 per group, with data combined from 2-4 independent experiments. *p<0.05, **p<0.01 by Kruskal-Wallis test, BJRiPS-CMs. Vertical line indicates that fetal and adult heart RNA were not included in statistical analysis due to lack of biological replicates (both were from single tubes purchased from commercial vendors). Error bars not shown on fetal and adult heart RNA due to lack of biological replicates. DMSO, dimethylsulfoxide; TBP, TATA-binding protein.

FIGS. 18A-18B provide evaluation of different time periods of Torin1 treatment. FIG. 18A shows mean fluorescence intensity of TNNT2-AlexaFluor647 after treatment with control (DMSO vehicle) or 200 nM Torin1 from days 8-11, 8-15, 9-11, 9-16, 11-14, or 11-18 of differentiation. n=3 per group, *p<0.05 by one-way ANOVA with Dunnett's multiple comparisons test to compare to control. FIG. 18B shows mean fluorescence intensity of Kir2.1 (extracellular) after treatment with control (DMSO vehicle) or 200 nM Torin1 from days 8-11, 8-15, 9-11, 9-16, 11-14, or 11-18 of differentiation. n=3 per group *p<0.05, **p<0.01, ****p<0.0001 by one-way ANOVA with Dunnett's multiple comparisons test to compare to control.

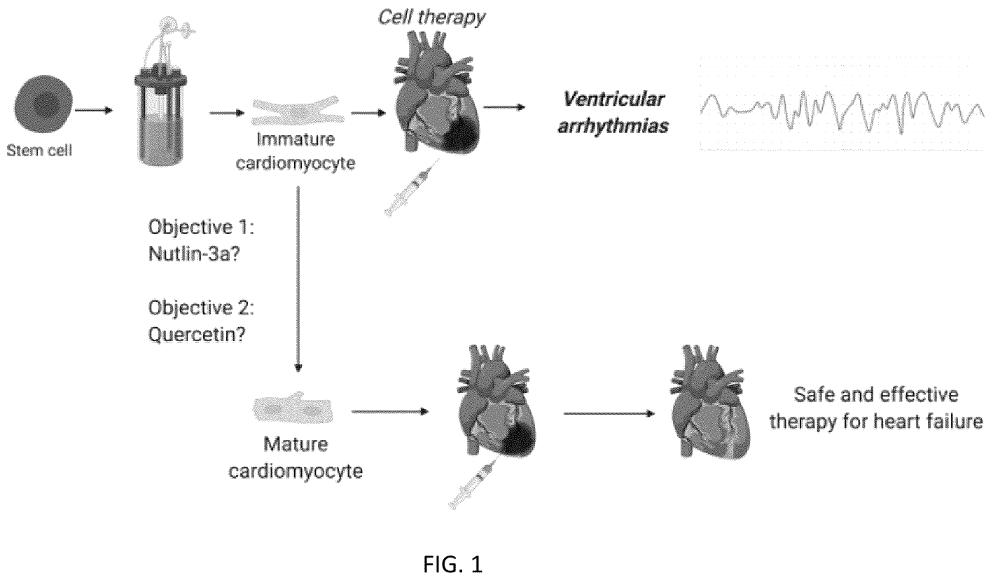

### DETAILED DESCRIPTION OF THE INVENTION

Current differentiation protocols to produce cardiomyocytes from human induced pluripotent stem cells (iPSCs) are capable of generating highly pure cardiomyocyte populations as determined by expression of cardiac troponin T. However, these cardiomyocytes remain immature, more closely resembling the fetal state, with a lower maximum contractile force, slower upstroke velocity, and immature mitochondrial function compared with adult cardiomyocytes. Immaturity of iPSC-derived cardiomyocytes may be a significant barrier to clinical translation of cardiomyocyte cell therapies for heart disease. During development, cardiomyocytes undergo a shift from a proliferative state in the fetus to a more mature but quiescent state after birth. The mechanistic target of rapamycin (mTOR) signaling pathway plays a key role in nutrient sensing and growth.

Cardiomyocytes were differentiated from iPSC lines using small molecules to modulate the Wnt pathway. In some embodiments, an upregulator of p53 is used at various time points and the contractile, metabolic, and electrophysiological properties of matured iPSC-derived cardiomyocytes were quantified. In some embodiments, an inhibitor of the mTOR pathway was used at various time points. In some embodiments, a small molecule inhibitor was used to inhibit p53 signaling. In some embodiments, the cardiomyocytes are differentiated and/or matured in a two-dimensional or three-dimensional culture medium.

Aspects of the disclosure relate to compositions, methods, kits, and agents for generating cardiomyocytes (referred to herein as non-naturally occurring cardiomyocytes, non-native cardiomyocytes, quiescent cardiomyocytes, or mature cardiomyocytes) from at least one stem cell, and mature or quiescent cardiomyocytes produced by those compositions, methods, kits, and agents for use in cell therapies, assays, and various methods of treatment.

The in vitro-produced cardiomyocytes generated according to the methods described herein demonstrate many advantages; for example, they are electrically mature (e.g., exhibit decreased automaticity), contractility mature, and metabolically mature. In addition, the generated cardiomyocytes may provide a new platform for cell therapy (e.g., transplantation into a subject in need of additional and/or functional cardiomyocytes) and research.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro.

As used herein, the term “adult cell” refers to a cell found throughout the body after embryonic development.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

As used herein “quiescence” or “cellular quiescence” is used to refer to a cellular resting state triggered by nutrient deprivation and is characterized by the ability to re-enter the cell cycle in response to appropriate stimuli. Quiescent cells retain metabolic and transcriptional activity. Cells can have varying depths of quiescence, including a transitional entry period into G₀, deep G₀, and a Gₐₗₑᵣₜ state, which is a more shallow state of quiescence during which cells are more responsive to stimuli triggering return to the cell cycle. Quiescent cardiomyocytes may exhibit expression of one or more quiescence markers, including p16 and p130.

The terms “endogenous cardiomyocyte” or “endogenous mature cardiomyocyte” are used herein to refer to a mature cardiomyocyte. A mature cardiomyocyte may exhibit electrical maturity, contractile maturity, and/or metabolic maturity. The phenotype of a cardiomyocyte is well known by persons of ordinary skill in the art, and includes, for example, ability to spontaneously beat, expression of markers such as cardiac troponin, TNNT2, TNNI3, myosin heavy chain, MYH6, MYH7, ryanodine receptor (RyR), sodium channel protein SCN5a, potassium voltage-gated channel KCNJ2, ATP2A2, PPARGC1a, Cx43, as well as distinct morphological characteristics such as organized sarcomeres, having rod shaped cells, and having T-tubules.

As used herein “cardiomyocyte,” “non-naturally occurring cardiomyocyte,” “non-native cardiomyocyte,” “quiescent cardiomyocyte,” and “mature cardiomyocyte,” all refer to cardiomyocytes produced by the methods as disclosed herein. The cardiomyocytes may be ventricular-, atrial-, and/or nodal-type cardiomyocytes, or a mixed population of cardiomyocytes. Cardiomyocytes may exhibit one or more features which may be shared with endogenous cardiomyocytes, including, but not limited to, capacity to beat spontaneously, are electrically mature, metabolically mature, contractility mature, exhibit appropriate expression of one or more gene markers (e.g., TNNI3, TNNT1, MYH6, MYH7, KCNJ2, RyR, and REST), exhibit appropriate expression of one or more quiescence markers (e.g., p16 and p130), exhibit appropriate morphological characteristics (e.g., rod shaped cells and organized sarcomeres), and expandability in culture. However non-naturally occurring cardiomyocytes are not identical to and are distinguishable from endogenous cardiomyocytes as described herein, including distinction on the basis of gene expression.

The term “cardiomyocyte marker” refers to, without limitation, proteins, peptides, nucleic acids, polymorphism of proteins and nucleic acids, splice variants, fragments of proteins or nucleic acids, elements, and other analytes which are specifically expressed or present in endogenous cardiomyocytes. Exemplary cardiomyocyte markers include, but are not limited to, cardiac troponin T (TNNT2), cardiac troponin I (TNNT3), potassium channel KCNJ2, repressor element-1 silencing transcription actor (REST), ryanodine receptor (RyR), sodium channel (SCN5a), and those described in Yang et al. Circ. Res. 2014; 114(3):511-23.

The term “immature cardiomyocyte” as used herein is meant a cardiomyocyte that is immature (e.g., electrical, metabolic, and/or contractile). Immature cardiomyocytes display automaticity or pacemaker-like activity, have a higher resting membrane potential and slower upstroke velocity, have a less organized sarcomere structure, and lower maximum contractile force, do not have T-tubules, predominantly acquire energy through glycolysis (rather than oxidative phosphorylation), and may be a senescent state rather than a quiescent state.

As used herein, the term “proliferation” means growth and division of cells. In some embodiments, the term “proliferation” as used herein in reference to cells refers to a group of cells that can increase in number over a period of time.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as a cardiomyocyte precursors), and then to an end-stage differentiated cell, which plays a characteristic role in a certain tissue type, and may or may not retain the capacity to proliferate further.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one immature cardiomyocyte or a precursor thereof with a maturation factor, or combination of maturation factors) is intended to include incubating the differentiation medium and/or agent and the cell together in vitro (e.g., adding the maturation factors to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one immature cardiomyocyte or a precursor thereof with a maturation factor as in the embodiments described herein can be conducted in any suitable manner. For example, the cells may be treated in three-dimensional culture. In some embodiments, the cells are treated in conditions that promote the formation of cardiomyocytes. The disclosure contemplates any conditions which promote the formation of mature cardiomyocytes. Examples of conditions that promote the formation of mature cardiomyocytes include, without limitation, suspension culture in low attachment tissue culture plates, spinner flasks, aggrewell plates. In some embodiments, the inventors have observed that mature cardiomyocytes have remained stable in media. In some aspects, serum (e.g., heat inactivated fetal bovine serum) is added prior to dissociating and re-plating the cells.

It is understood that the cells contacted with a maturation factor (e.g., a cardiomyocyte maturation factor) can also be simultaneously or subsequently contacted with another agent, such as other differentiation agents or environments to stabilize the cells, or to differentiate or mature the cells further.

Similarly, at least one immature cardiomyocyte or a precursor thereof can be contacted with at least one cardiomyocyte maturation factor and then contacted with at least another cardiomyocyte maturation factor. In some embodiments, the cell is contacted with at least one cardiomyocyte maturation factor, and the contact is temporally separated, and in some embodiments, a cell is contacted with at least one cardiomyocyte maturation factor substantially simultaneously. In some embodiments, the cell is contacted with at least two, at least three, at least four, at least five, at least six, at least seven, at least eight, at least nine, or at least 10 cardiomyocyte maturation factors

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a cardiomyocyte described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of cardiomyocytes, refers to a population of cells that contain fewer than about 20%, more preferably fewer than about 15%, 10%, 8%, 7%, most preferably fewer than about 5%, 4%, 3%, 2%, 1%, or less than 1%, of cells that are not cardiomyocytes as defined by the terms herein. In some embodiments, the present invention encompasses methods to expand a population of cardiomyocytes, wherein the expanded population of cardiomyocytes is a substantially pure population of cardiomyocytes.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

As used herein, the term “DNA” is defined as deoxyribonucleic acid.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate or dedifferentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms “treat”, “treating”, “treatment”, etc. refer to providing medical or surgical attention, care, or management to an individual. The individual is usually ill or injured, or at increased risk of becoming ill relative to an average member of the population and in need of such attention, care, or management. It may include administering to a subject an effective amount of a composition so that the subject exhibits a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. The term “treatment” includes prophylaxis. Those in need of treatment include those already diagnosed with a condition (e.g., muscle disorder or disease), as well as those likely to develop a condition due to genetic susceptibility or other factors.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing cardiomyocytes (e.g., mature cardiomyocytes) or precursors thereof, germ cells may be used in place of, or with, the stem cells to provide at least one cardiomyocyte, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006). One possible application of ES cells is to generate new cardiomyocytes for the cell replacement therapy of heart failure (e.g., chronic heart failure), by first producing cardiac progenitors, from, e.g., hESCs, and then further differentiating the cardiac progenitors into at least one immature cardiomyocyte or precursor thereof, and then further differentiating the at least one immature cardiomyocyte or precursor thereof into a cardiomyocyte (e.g., mature cardiomyocyte).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad 7-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one cardiomyocyte maturation factor according to the methods as disclosed herein, whereas in other examples it may be desirable to differentiate the stem cells to one or more intermediate cell types prior to exposure of the at least one cardiomyocyte maturation factor (s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature cardiomyocytes did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497; Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic microenvironment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

### Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Alternatively, pluripotent SCs can be maintained in an undifferentiated state even without feeder cells. The environment for feeder-free cultures includes a suitable culture substrate, particularly an extracellular matrix such as MATRIGEL® (gelatinous protein mixture) or laminin. Typically, enzymatic digestion is halted before cells become completely dispersed (˜5 min with collagenase IV). Clumps of ˜10 to 2,000 cells are then plated directly onto the substrate without further dispersal.

### Generating Cardiomyocytes

Aspects of the disclosure relate to generating cardiomyocytes (e.g., mature, quiescent cardiomyocytes). Generally, the cardiomyocytes produced according to the methods disclosed herein demonstrate several hallmarks of functional mature, quiescent cardiomyocytes, including, but not limited to, being electrically mature (e.g., exhibit decreased automaticity), contractility mature, and metabolically mature.

The cardiomyocytes can be produced according to any suitable culturing protocol or series of culturing protocols to differentiate a stem cell or pluripotent cell to a desired stage of differentiation. In some embodiments, the cardiomyocytes or the precursors thereof are produced by culturing at least one pluripotent cell for a period of time and under conditions suitable for the at least one pluripotent cell to differentiate into the cardiomyocytes or the precursors thereof. In some embodiments, the cardiomyocytes are produced by shifting an immature cardiomyocyte from a senescent state to a quiescent state, thereby enhancing maturation of the cardiomyocytes.

In some embodiments, the cardiomyocytes are a substantially pure population of cardiomyocytes. In some embodiments, a population of cardiomyocytes or precursors thereof comprises a mixture of pluripotent cells or differentiated cells. In some embodiments, a population of cardiomyocytes or precursors thereof is substantially free or devoid of embryonic stem cells or pluripotent cells or iPS cells.

In some embodiments, a somatic cell, e.g., a fibroblast, can be isolated from a subject, for example as a tissue biopsy, such as, for example, a skin biopsy, and reprogrammed into an induced pluripotent stem cell for further differentiation to produce a cardiomyocyte or precursor thereof for use in the compositions and methods described herein. In some embodiments, a somatic cell, e.g., a fibroblast, is maintained in culture by methods known by one of ordinary skill in the art, and in some embodiments, propagated prior to being converted into cardiomyocytes by the methods as disclosed herein.

In some embodiments, the cardiomyocytes or precursors thereof are maintained in culture by methods known by one of ordinary skill in the art, and in some embodiments, propagated prior to being converted into cardiomyocytes by the methods as disclosed herein.

Further, cardiomyocytes or precursors thereof, e.g., immature cardiomyocytes, can be from any mammalian species, with non-limiting examples including a murine, bovine, simian, porcine, equine, ovine, or human cell. For clarity and simplicity, the description of the methods herein refers to a mammalian cardiomyocytes or precursor thereof, but it should be understood that all of the methods described herein can be readily applied to other cell types of cardiomyocytes or precursors thereof. In some embodiments, the cardiomyocytes or precursors thereof are derived from a human individual.

Aspects of the disclosure involve immature, senescent cardiomyocytes. Immature cardiomyocytes of use herein can be derived from any source or generated in accordance with any suitable protocol. In some aspects, pluripotent stem cells, e.g., iPSCs or hESCs, are differentiated to immature cardiomyocytes. In some aspects, the immature cardiomyocytes are further matured to mature cardiomyocytes. In some embodiments, pluripotent stem cells are differentiated to immature cardiomyocytes using a differentiation protocol described by Lian et al. (Nat Protoc. 2012; 8(1): 162-175), which is incorporated herein by reference. In some embodiments, the differentiation protocol described by Lian was modified as described herein. In some embodiments, pluripotent stem cells are contacted with one or more small molecules to manipulate the Wnt pathway, and thereby differentiating the pluripotent stem cells into immature cardiomyocytes. In some aspects, the one or more small molecules are selected from the group consisting of CHIR 99021 and IWP4. In some embodiments, a population of pluripotent stem cells is contacted with a first Wnt pathway modulator (e.g., CHIR 99021), and is then contacted with a second Wnt pathway modulator (e.g., IWP4).

Aspects of the disclosure involve cardiomyocytes (e.g., mature cardiomyocytes). Cardiomyocytes of use herein can be derived from any source or generated in accordance with any suitable protocol. In some aspects, senescent cardiomyocytes (e.g., immature cardiomyocytes) are induced into quiescent cardiomyocytes (e.g., mature cardiomyocytes). Cellular quiescence may facilitate cardiomyocyte maturation. In some aspects, immature cardiomyocytes are induced to mature into mature cardiomyocytes.

In some aspects, the disclosure provides a method for inducing quiescence in cardiomyocytes (e.g., shifting cardiomyocytes to a quiescent state). In certain aspects, inducing quiescence in cardiomyocytes enhances maturation of the cardiomyocytes. In some embodiments, cardiomyocytes are shifted to a quiescent state by upregulating expression of a cell cycle regulator in the cardiomyocytes. In certain embodiments, cardiomyocytes are shifted to a quiescent state by upregulating expression of p53 in the cardiomyocytes. Expression of p53 may be upregulated in cardiomyocytes by contacting the cardiomyocytes with an activator of p53. In some embodiments, an activator of p53 is a small molecule. In certain embodiments, an activator of p53 is Torin1. In certain embodiments, an activator of p53 is not Torin1. In some embodiments, an activator of p53 is an inhibitor of MDM2. In certain embodiments, an activator of p53 is nutlin-3a. In some embodiments, an activator of p53 is a senolytic. In certain embodiments, an activator of p53 is quercetin. In some embodiments, an activator of p53 is a combination of two or more agents.

In some embodiments, cardiomyocytes are shifted to a quiescent state by inhibiting mTOR signaling and upregulating expression of p53. In some embodiments, cardiomyocytes are shifted to a quiescent state by upregulating expression of p53 and not inhibiting mTOR signaling. In some aspects, mTOR signaling may be inhibited in cardiomyocytes by contacting the cardiomyocytes with an inhibitor of mTORC1 and mTORC2. In some embodiments, the inhibitor of mTORC1 and mTORC2 is Torin1. In some embodiments, the inhibitor of mTORC1 and mTORC2 is not Torin1. In certain embodiments, p53 expression is upregulated and mTOR signaling is inhibited by contacting cardiomyocytes with a single agent. In certain embodiments, the single agent is Torin1. In other embodiments, the single agent is not Torin1.

In some aspects, the disclosure provides a method for generating mature cardiomyocytes (e.g., electrically mature, contractility mature, and/or metabolically mature) from immature cardiomyocytes, the method comprising inducing quiescence in the cardiomyocytes (i.e., shifting cardiomyocytes towards a quiescent state, i.e., a mature state). In some embodiments, the cardiomyocytes are shifted towards a quiescent state (e.g., a mature state) by upregulating p53 expression. The cardiomyocytes may be shifted towards a quiescent state by contacting the immature cardiomyocytes with an activator of p53 (e.g., nutlin-3a or quercetin). In some embodiments, the cardiomyocytes are shifted towards a quiescent state (e.g., a mature state) by upregulating p53 expression and downregulating mTOR signaling. In some embodiments, the cardiomyocytes are shifted towards a quiescent state by upregulating p53 expression and not downregulating mTOR signaling. In some embodiments, the cardiomyocytes are shifted towards a quiescent state by contacting immature, senescent cardiomyocytes with Torin1. In some embodiments, the cardiomyocytes are shifted towards a quiescent state by contacting immature, senescent cardiomyocytes with an agent that is not Torin1.

In some aspects, the disclosure provides a method for generating mature cardiomyocytes (e.g., electrically mature, contractility mature, and/or metabolically mature) from immature cardiomyocytes, the method comprising contacting a population of cells comprising immature cardiomyocytes with at least one cardiomyocyte maturation factor comprising a p53 activator and/or an inhibitor of mTOR, to induce the maturation (e.g., in vitro maturation) of at least one immature cardiomyocyte in the population into a cardiomyocyte. In some embodiments, a population of cells comprising immature cardiomyocytes is contacted with at least one cardiomyocyte maturation factor (e.g., p53 activator and/or mTOR inhibitor). In some aspects, p53 expression is upregulated in combination with mTOR inhibition to enhance the maturation of cardiomyocytes derived from stem cells. In some aspects, p53 expression is upregulated without inhibiting mTOR to enhance the maturation of cardiomyocytes derived from stem cells.

The disclosure contemplates the use of any p53 activator that encourages immature cardiomyocytes to shift from a senescent state to a quiescent state (e.g., differentiate and/or mature into cardiomyocytes (e.g., alone or in combination with another cardiomyocyte maturation factor (e.g., an mTOR inhibitor))). In some embodiments, the p53 activator is an upregulator of p53 expression. Upregulation of p53 may include an increase in total p53 and phosphorylated p53 protein. Examples of p53 activators or upregulators include small molecule, nucleic acid, amino acid, metabolite, polypeptide, antibody and antibody-like molecules, aptamers, macrocycles, and other molecules. In some aspects, an upregulator of p53 is an MDM2 inhibitor. In certain aspects, an upregulator of p53 is nutlin-3a. In some aspects, an upregulator of p53 is a senolytic. In certain aspects, an upregulator of p53 is quercetin. In some aspects, an upregulator of p53 is Torin1. In some aspects, an upregulator of p53 is an agent that is not Torin1. In some aspects, an upregulator of p53 is an agent that is not an mTOR inhibitor. In some aspects, an upregulator of p53 is a combination of nutlin-3a and Torin1. In some aspects, an upregulator of p53 is a combination of nutlin-3a and quercetin. In some aspects, an upregulator of p53 is a combination of nutlin-3a, quercetin, and Torin1.

In some aspects, the disclosure provides a method for generating mature cardiomyocytes (e.g., electrically mature, contractility mature, and/or metabolically mature) from immature cardiomyocytes, the method comprising contacting a population of cells comprising immature cardiomyocytes with at least one cardiomyocyte maturation factor comprising an mTOR inhibitor, to induce the maturation (e.g., in vitro maturation) of at least one immature cardiomyocyte in the population into a cardiomyocyte. In some embodiments, a population of cells comprising immature cardiomyocytes is contacted with at least one cardiomyocyte maturation factor (e.g., mTOR inhibitor, PI3K inhibitor, or Akt inhibitor). In some aspects, the PI3K/Akt/mTOR pathway is manipulated (e.g., inhibited) to enhance the maturation of cardiomyocytes derived from stem cells.

The disclosure contemplates the use of any mTOR inhibitor that encourages immature cardiomyocytes to differentiate and/or mature into cardiomyocytes (e.g., alone or in combination with another cardiomyocyte maturation factor (e.g., a p53 upregulator)). In some embodiments, mTOR comprises mTORC1 and/or mTORC2. In some embodiments, the mTOR inhibitor is an inhibitor of mTORC1 and/or mTORC2. In some embodiments, the mTOR inhibitor inhibits phosphorylation of 4E-BP1. Inhibiting phosphorylation of 4E-BP1 may affect regulation of the oxidative phosphorylation pathway. Inhibiting phosphorylation of 4E-BP1 may degrade p21 and thereby upregulate p53. Non-limiting examples of modulators of the oxidative phosphorylation pathway include 4EGI-1, JR-AB2-011 (an mTORC2 inhibitor), AICAR (an AMPK activator), metformin (an AMPK activator and mTORC1/2 inhibitor), and HLM006474 (an E2F inhibitor). In some embodiments, the mTOR inhibitor inhibits phosphorylation of 4E-BP1 and Ribosomal protein S6. In some embodiments, the mTOR inhibitor comprises Torin1, Torin2, rapamycin, everolimus, and/or temsirolimus. In certain embodiments, a population of cells comprising immature cardiomyocytes is contacted with Torin1, to induce the maturation of at least one immature cardiomyocyte in the population into a cardiomyocyte (e.g., a mature cardiomyocyte). In some embodiments, a population of cells comprising immature cardiomyocytes is contacted with Torin2, to induce the maturation of at least one immature cardiomyocyte in the population into a cardiomyocyte (e.g., a mature cardiomyocyte).

In some embodiments, contacting may be performed by maintaining the at least one immature cardiomyocyte or a precursor thereof in culture medium comprising the one or more cardiomyocyte maturation factors. In some embodiments, the contacting is performed by maintaining the at least one immature cardiomyocyte or a precursor thereof in two-dimensional (2D) culture medium comprising the one or more cardiomyocyte maturation factors. In other embodiments, the contacting is performed by maintaining the at least one immature cardiomyocyte or a precursor thereof in three-dimensional (3D) culture medium comprising the one or more cardiomyocyte maturation factors. In some aspects, the one or more cardiomyocyte maturation factors are applied to the culture medium (e.g., the 2D or 3D culture medium) with a pulse treatment. In some embodiments, pulse treatment occurs for 1 to 24 hours, 5 to 18 hours, or 10 to 12 hours. In some embodiments, pulse treatment occurs for 1 hour or longer. In some embodiments, pulse treatment occurs for 24 hours or less. In some embodiments, pulse treatment occurs for a period of 1 to 5 or 2 to 3 days. In one embodiment, a pulse treatment occurs at a pre-determined time and for a pre-determined length of time for 2-3 days, thereby mimicking a circadian cycle. In some aspects, the one or more cardiomyocyte maturation factors are applied to the culture medium (e.g., the 2D or 3D culture medium) with a continuous treatment.

In some embodiments at least one immature cardiomyocyte or a precursor thereof can be genetically engineered. In some embodiments, at least one immature cardiomyocyte or a precursor thereof can be genetically engineered to express one or more cardiomyocyte (e.g., mature cardiomyocyte) markers as disclosed herein, for example express at least one polypeptide selected from TNNI3, KCNJ2, REST/NRSF, Ryr, and SCN5a, or an amino acid sequence substantially homologous thereof, or functional fragments or functional variants thereof.

Where the immature cardiomyocytes or precursors thereof are maintained under in vitro conditions, conventional tissue culture conditions and methods can be used, and are known to those of skill in the art. Isolation and culture methods for various cells are well within the abilities of one skilled in the art.

In the methods of the disclosure at least one cardiomyocyte or a precursor thereof can, in general, be cultured under standard conditions of temperature, pH, and other environmental conditions, e.g., as adherent cells in tissue culture plates or in 3D culture in Erlenmeyer flasks at 37° C. in an atmosphere containing 5-10% CO₂. The cells and/or the culture medium are appropriately modified to achieve conversion to cardiomyocytes as described herein.

In certain examples, the cardiomyocyte maturation factors can be used to induce the differentiation of at least one immature cardiomyocyte or precursor thereof by exposing or contacting at least one immature cardiomyocyte or precursor thereof with an effective amount of a cardiomyocyte maturation factor described herein to differentiate the at least one immature cardiomyocyte or precursor thereof into at least one cardiomyocyte (e.g., a mature cardiomyocyte). In some aspects, the exposing or contacting of the immature cardiomyocyte with a cardiomyocyte maturation factor occurs continuously, or in other aspects, occurs via a pulse treatment.

Accordingly, included herein are cells and compositions made by the methods described herein. The exact amount and type of cardiomyocyte maturation factor can vary depending on the number of immature cardiomyocytes or precursors thereof, the desired differentiation stage and the number of prior differentiation stages that have been performed.

In certain examples, a cardiomyocyte maturation factor is present in an effective amount. As used herein, “effective amount” refers to the amount of the compound that should be present for the differentiation of at least 10% or at least 20% or at least 30% of the cells in a population of immature cardiomyocytes or precursors thereof into cardiomyocytes.

In additional examples, cardiomyocyte maturation factors can be present in the culture medium of the at least one immature cardiomyocyte or precursor thereof, or alternatively, the cardiomyocyte maturation factors may be added to the at least one immature cardiomyocytes or precursor thereof during some stage of growth.

In some embodiments, immature cardiomyocytes are contacted with a cardiomyocyte maturation factor (e.g., an mTOR inhibitor and/or p53 upregulator) after the immature cardiomyocytes begin beating. In some aspects, immature cardiomyocytes are beating for a period of 1 to 5 days, 1 to 4 days, 1 to 3 days, 1 to 2 days, 1 day, 2 days, 3 days, 4 days, or 5 days before being contacted with a cardiomyocyte maturation factor. In some aspects, immature cardiomyocytes are beating for a period of 1 to 40 days, 2 to 35 days, 3 to 30 days, 4 to 25 days, 5 to 20 days, 7 to 35 days, 14 to 30 days, or 21 to 28 days before being contacted with a cardiomyocyte maturation factor. In some aspects immature cardiomyocytes are not contacted with a cardiomyocyte maturation factor (e.g., an mTOR inhibitor and/or p53 upregulator) if the immature cardiomyocytes have not begun beating.

In some embodiments, immature cardiomyocytes are contacted with a cardiomyocyte maturation factor (e.g., an mTOR inhibitor and/or p53 upregulator) after the immature cardiomyocytes begin expressing troponin T (TNNT2) and myosin heavy chain 6 (MYH6). In some embodiments, immature cardiomyocytes are contacted with a cardiomyocyte maturation factor (e.g., an mTOR inhibitor and/or p53 upregulator) after the immature cardiomyocytes begin expressing troponin T (TNNT2), troponin I (TNNI3), myosin heavy chain 6 (MYH6), and myosin heavy chain 7 (MYH7).

Where the at least one immature cardiomyocyte or a precursor thereof is maintained under in vitro conditions, conventional tissue culture conditions and methods can be used, and are known to those of skill in the art. Isolation and culture methods for various cells are well within the abilities of one skilled in the art.

In some aspects, pluripotent stem cells are cultured in RPMI+B27 and are contacted with a GSK3 inhibitor/WNT activator (e.g., CHIR 99021) at Day 0 of a differentiation protocol. At Day 2 of the protocol the WNT activator (e.g., CHIR 99021) is removed. At Day 3 of the protocol a WNT inhibitor (e.g., IWP4) is added and at D5 the WNT inhibitor (e.g., IWP4) is removed. At Day 7 insulin is added to the culture and ever 2-3 days the media is changed. At Day 11 an mTOR inhibitor (e.g., Torin1) is added and is maintained in the culture until Day 18. At the completion of the differentiation protocol, mature cardiomyocytes are obtained from the culture media.

In some aspects, pluripotent stem cells are cultured in RPMI+B27 and are contacted with a GSK3 inhibitor/WNT activator (e.g., CHIR 99021) from days 0 to 2 of the differentiation protocol. From days 2 to 4 of the protocol a WNT inhibitor (e.g., IWP4) is added. At Day 7 insulin is added to the culture and ever 2-3 days the media is changed. Upon beating, cardiomyocytes were treated with Torin1 beginning at approximately 2 days after onset of beating for a period of 7 days. In alternative aspects, upon beating, cardiomyocytes were treated with a cardiomyocyte maturation factor that was not Torin1 (e.g., nutlin-3a and/or quercetin) beginning at approximately 2 days after onset of beating for a period of 5 days. Upon completion of treatment, media was switched back to RPMI/B27/insulin and maintained with media change every 2-3 days. At the completion of the differentiation protocol, mature cardiomyocytes are obtained from the culture media.

In some embodiments, the differentiation protocol for obtaining cardiomyocytes from immature cardiomyocytes or precursors thereof occurs in a two-dimensional culture system. In some embodiments, the differentiation protocol for obtaining cardiomyocytes from immature cardiomyocytes or precursors thereof occurs in a three-dimensional culture system (e.g., using a 3D bioreactor system).

Aspects of the disclosure involve generating cardiomyocytes which resemble endogenous mature cardiomyocytes in form and function, but nevertheless are distinct from native cardiomyocytes. In some embodiments, the morphology of the cardiomyocytes resembles the morphology of endogenous cardiomyocytes. In some embodiments, the cardiomyocytes are quiescent. In some embodiments, the cardiomyocytes exhibit increased expression of quiescence markers (e.g., p16, p53, and p130). In some embodiments, the cardiomyocytes exhibit decreased expression of proliferative markers (e.g., cyclin C1, E2F1, and Ki67). In some embodiments, the cardiomyocytes exhibit increased expression of inhibitor E2F factors (e.g., E2F3b-8). In some embodiments, the cardiomyocytes exhibit decreased expression of stimulatory E2F factors (e.g., E2F1-3a).

In some embodiments, the cardiomyocytes are mature. In some embodiments, the cardiomyocytes exhibit increased expression of sarcomeric proteins (e.g., TNNT2, TNNI3, MYH6, and/or MYH7). In some embodiments, the cardiomyocytes exhibit decreased beating rate as compared to fetal or immature cardiomyocytes. In some embodiments, the cardiomyocytes exhibit increased expression of ion channels (e.g., KCNJ2, HCN4, SCN5a, RYR2, CACNA1c, and/or SERCA2a (ATP2a2)). In some embodiments, the cardiomyocytes exhibit increased expression of brain natriuretic peptide. In some embodiments, the cardiomyocytes exhibit increased expression of a nuclear transcription factor (e.g., REST/NRSF, GATA4, TBX20, BRG1, YAP, ERBB2, PITX2, MEIS1, ATA4, Nkx2-5, TBX5, NFAT, TEAD, HAND, CHF1, Fox03/FoxO4, CHF1/Hey2, CHF1/Hey2, Fox01, Mef2C, SRF, p53, NFkB, and combinations thereof). In some embodiments, the cardiomyocytes exhibit increased expression of a regulator of the oxidative phosphorylation pathway (e.g., PGC1-alpha).

Generating cardiomyocytes by conversion or maturation of at least one immature cardiomyocyte or a precursor thereof using the methods of the disclosure has a number of advantages. First, the methods of the disclosure allow one to generate autologous cardiomyocytes, which are cell specific to and genetically matched with an individual. In general, autologous cells are less likely than non-autologous cells to be subject to immunological rejection. The cells are derived from at least one immature cardiomyocyte or a precursor thereof, e.g., a cardiac progenitor obtained by reprogramming a somatic cell (e.g., a fibroblast) from the individual to an induced pluripotent state, and then culturing the pluripotent cells to differentiate at least some of the pluripotent cells to at least one immature cardiomyocyte or precursor, followed by the induced maturation in vitro of the at least one immature cardiomyocyte into a cardiomyocyte (e.g., a mature cardiomyocyte).

In some embodiments, a subject from which at least one immature cardiomyocyte or precursor thereof are obtained is a mammalian subject, such as a human subject. In some embodiments, the subject is suffering from a cardiac disorder. In some embodiments, the subject is suffering from chronic heart failure. In some embodiments, the subject is suffering from ventricular arrhythmias. In such embodiments, the at least one immature cardiomyocyte or precursor thereof can be differentiated into a cardiomyocyte ex vivo by the methods as described herein and then administered to the subject from which the cells were harvested in a method to treat the subject for the cardiac disorder (e.g., heart failure).

In some embodiments, at least one immature cardiomyocyte or a precursor thereof is located within a subject (in vivo) and is converted to become a cardiomyocyte by the methods as disclosed herein in vivo. In some embodiments, conversion of at least one immature cardiomyocyte or a precursor thereof to a cardiomyocyte in vivo can be achieved by administering to a subject a composition comprising at least one, at least two, at least three, at least four, or more cardiomyocyte maturation factors as described herein. In some embodiments, conversion of at least one immature cardiomyocyte or a precursor thereof to a cardiomyocyte in vivo can be achieved by administering to a subject a composition comprising at least one, at least two, at least three, or at least four cardiomyocyte maturation factors as described herein.

### Cardiomyocytes

In some embodiments, the disclosure provides mature cardiomyocytes. The cardiomyocytes disclosed herein share many distinguishing features of native cardiomyocytes, but are different in certain aspects (e.g., gene expression profiles). In some embodiments, the cardiomyocyte is non-native or non-naturally occurring. As used herein, “non-native” or “non-naturally occurring” means that the cardiomyocytes are markedly different in certain aspects from cardiomyocytes which exist in nature, i.e., native cardiomyocytes. It should be appreciated, however, that these marked differences typically pertain to structural features which may result in the cardiomyocytes exhibiting certain functional differences, e.g., although the gene expression patterns of cardiomyocytes differs from native cardiomyocytes, the cardiomyocytes behave in a similar manner to native cardiomyocytes but certain functions may be altered (e.g., improved) compared to native cardiomyocytes.

The cardiomyocytes of the disclosure share many characteristic features of native cardiomyocytes which are important for normal cardiomyocyte function. Characteristics of mature cardiomyocytes are described in Yang et al. Circ. Res. 2014; 114(3):511-23.

In some embodiments, the cardiomyocytes are quiescent. In some embodiments, cardiomyocytes retain metabolic and transcriptional activity in the quiescent state. In some embodiments, the quiescent state facilitates cardiomyocyte maturation. In some embodiments, cardiomyocytes express, or express at an increased level (i.e., compared to a control) certain quiescent markers, including p16, p53, and p130. In some embodiments, cardiomyocytes do not express, or express at a reduced level (i.e., compared to a control), proliferative markers, such as Ki67, cyclin C1, and E2F1. In some embodiments, cardiomyocytes exhibit increased expression of inhibitory E2F factors (e.g., E2F3b, E2F4, E2F5, E2F6, E2F7, and E2F8). In some embodiments, the cardiomyocytes exhibit decreased expression of stimulatory E2F factors (e.g., E2F1, E2F2, and E2F3).

In some embodiments, the cardiomyocytes are electrically mature cardiomyocytes. In some embodiments, the cardiomyocytes exhibit decreased automaticity. Native mature adult human cardiomyocytes beat at 20-30 beats per minute naturally. In some aspects, the cardiomyocytes described herein exhibit a slower intrinsic beating rate. In some embodiments, the cardiomyocytes beat at 15 to 35 beats per minute, 15 to 20 beats per minute, or 30 to 35 beats per minute. In certain embodiments, the cardiomyocytes exhibit a spontaneous beating rate of less than 40 beats per minute. Slower intrinsic beating rate may suggest decreased automaticity, and cardiomyocytes with decreased automaticity (i.e., decreased drive to beat spontaneously) may decrease the risk of arrhythmias in cell therapy.

In some embodiments, the cardiomyocytes are contractility mature cardiomyocytes. In some embodiments, the cardiomyocytes exhibit increased RNA and protein expression of contractile proteins (e.g., sarcomeric contractile proteins) (i.e., as compared to immature cardiomyocytes). In some aspects, the cardiomyocytes exhibit increased RNA and protein expression of at least one of cardiac troponin T (TNNT2), cardiac troponin I (TNNI3), myosin heavy chain protein 6 (MYH6), and myosin heavy chain protein 7 (MYH7). In some embodiments, the cardiomyocytes exhibit increased RNA expression of the proteins in a dose-dependent manner (e.g., upon treating an immature cardiomyocyte with an mTOR inhibitor). Increased expression of one or more sarcomeric proteins may enhance contractility of the cardiomyocytes. In some aspects, cardiomyocytes exhibit increased overall content or amount of TNNI3, and in some aspects increased TNNI3 content or amount relative to the slow skeletal form of TNNI3. In some aspects, cardiomyocytes exhibit increased MYH7 content or protein relative to MYH6 content or protein (e.g., in humans). In some embodiments, TNNI3 expression in the cardiomyocytes is increased by nearly 5-fold, 6-fold, 7-fold, 8-fold, 9-fold, 10-fold, 11-fold, 12-fold, 13-fold, 14-fold, or 15-fold as compared to an immature cardiomyocyte. In some aspects, contractility of cardiomyocytes may be measured by measuring cell motion (e.g., using one or more imaging methods). A soft substrate that will bend with cardiomyocyte contraction may be used in conjunction with one or more imaging methods to quantify contractility. In some aspects, a cardiomyocyte has increased contractility as compared to an immature cardiomyocyte.

In some embodiments, the cardiomyocytes are metabolically mature cardiomyocytes. In some aspects, a cardiomyocyte has increased metabolic activity as compared to an immature cardiomyocyte. In some embodiments, a cardiomyocyte has increased oxygen consumption and/or extracellular acidification rate as compared to immature cardiomyocytes. Metabolic maturity may be quantified using a Seahorse mito stress metabolic assay (Agilent). The assay may be used to measure oxygen consumption rate and extracellular acidification rate in response to one or more compounds (e.g., small molecule compounds) that affect mitochondrial function.

In some embodiments, the cardiomyocytes exhibit a morphology that resembles the morphology of an endogenous mature cardiomyocyte. In some embodiments, the cardiomyocytes form rod-shaped cells. In some embodiments, the cardiomyocytes exhibit an organized sarcomere structure. In some aspects, the average sarcomere length is 1.0 to 4.0 μm, 1.5 to 3.5 μm, or 2.0 to 3.0 μm. In some aspects, the average sarcomere length is about 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, or 3.0 μm.

In some aspects the cardiomyocytes exhibit a mature ion channel expression profile. In some embodiments, the cardiomyocytes exhibit increased ion channel expression (i.e., compared to immature cardiomyocytes). Ion channel expression may be increased for one or more of KCNJ2, HCN4, SCN5a, RYR2, CACNA1c, or SERCA2a (ATP2a2). In some aspects, KCNJ2 expression in the cardiomyocytes increases in a dose-dependent manner (e.g., upon treating an immature cardiomyocyte with one or more cardiomyocyte maturation factors in 2D or 3D culture). In some embodiments, the cardiomyocytes exhibit increased expression of SCN5a, KCNJ2, and RYR2 (i.e., compared to immature cardiomyocytes). In some embodiments, the resting membrane potential of cardiomyocytes is within the range of −70 to −150 mV, −75 to −125 mV, −80 to −100 mV, or −85 to −95 mV. In some embodiments, the resting membrane potential of the cardiomyocytes is about −85 mV, −86 mV, −87 mV, −88 mV, −89 mV, −90 mV, −91 mV, −92 mV, −93 mV, −94 mV, or −95 mV. In certain embodiments, the resting membrane potential of the cardiomyocytes is less than or equal to −70 mV. In some embodiments, the upstroke velocity of the cardiomyocytes is within the range of 150 to 350 V/sec, 175 to 325 V/sec, 200 to 300 V/sec, or 225 to 275 V/sec. In some embodiments, the upstroke velocity of the cardiomyocytes is about 200 V/sec, 210 V/sec, 220 V/sec, 230 V/sec, 240 V/sec, 250 V/sec, 260 V/sec, 270 V/sec, 280 V/sec, 290 V/sec, or 300 V/sec. In certain embodiments, the upstroke velocity of the cardiomyocytes is greater than 200 V/sec, e.g., about 250 V/sec.

In some embodiments, the cardiomyocytes exhibit increased expression of a transcription factor (e.g., a nuclear transcription factor). In some embodiments, the cardiomyocytes exhibit increased expression of one or more transcription factors. In some embodiments, the cardiomyocytes exhibit increased expression of nuclear transcription factor repressor element-1 silencing transcription factor (REST), also known as neuron restrictive silencer factor (i.e., as compared to immature cardiomyocytes). In some aspects the expression of REST, suppresses expression of HCN4. In some embodiments, the cardiomyocytes exhibit increased expression of REST in a dose-dependent manner (e.g., upon treating an immature cardiomyocyte with one or more cardiomyocyte maturation factors in 2D or 3D culture). In some embodiments, the cardiomyocytes exhibit increased expression of transcription factors selected from the group consisting of REST/NRSF, GATA4, TBX20, BRG1, YAP, ERBB2, PITX2, MEIS1, ATA4, Nkx2-5, TBX5, NFAT, TEAD, HAND, CHF1, FoxO3/FoxO4, CHF1/Hey2, CHF1/Hey2, FoxO1, Mef2C, SRF, p53, NFkB, and combinations thereof.

In some embodiments, the cardiomyocytes exhibit increased expression of a regulator of an oxidative phosphorylation pathway (i.e., as compared to an immature cardiomyocyte). In some embodiments, the cardiomyocytes exhibit increased expression of a regulator of an oxidative phosphorylation pathway PGC1-alpha (PPARGC1a). Mature cardiomyocytes switch from deriving energy from glycolysis to oxidative phosphorylation. Upregulation of PGClalpha may enhance pathways associated with oxidative phosphorylation. In some embodiments, metabolism occurs predominantly from fatty acids, for example from fatty acid β-oxidation (e.g., instead of glycolysis).

In some embodiments, the cardiomyocytes exhibit increased expression of a brain natriuretic peptide (BNP) (i.e., as compared to an immature cardiomyocyte). In some embodiments, the cardiomyocytes exhibit increased expression of natriuretic peptide B (NPPB).

In some embodiments, the cardiomyocytes exhibit increased mitochondrial content (i.e., as compared to immature cardiomyocytes). For example, the cardiomyocytes may have increased mitochondria length and increased mitochondrial membrane potential. In some embodiments, mitochondria occupy about 5 to 70%, 10 to 60%, 15 to 50%, or 20 to 40% of the cardiomyocyte by volume. In some aspects, the mitochondria are distributed throughout the cardiomyocyte in a crystal-like lattice pattern.

In some embodiments, the cardiomyocytes have a conduction velocity of about 0.15 to 2.5 meters/sec, 0.2 to 2.0 meters/sec, 0.25 to 1.5 meters/sec, or 0.3 to 1.0 meters/sec. In some embodiments, the cardiomyocytes have a conduction velocity of about 0.3, 0.4, 0.5, 0.6, 0.7, 0.8, 0.9, or 1.0 meters/sec.

In some embodiments, the cardiomyocytes express at least one marker characteristic of an endogenous mature cardiomyocyte selected from the group consisting of TNNT2, TNNI3, MYH7, MYH6, KCNJ2, HCN4, SCN5a, RYR2, CACNA1c, SERCA2a (ATP2a2), NPPB (BNP), REST, and PPARGC1a (PGC1a). In some embodiments, the cardiomyocytes express at least one marker characteristic of an endogenous mature cardiomyocyte selected from the group consisting of TNNT2, TNNI3, KCNJ2, REST, RyR, and SCN5a.

The cardiomyocytes are differentiated in vitro from any starting cell as the invention is not intended to be limited by the starting cell from which the cardiomyocytes are derived. Exemplary starting cells include, without limitation, immature cardiomyocytes or any precursor thereof such as a cardiac progenitor cell, a pluripotent stem cell, an embryonic stem cell, and induced pluripotent stem cell. In some embodiments, the cardiomyocytes are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), or a transdifferentiated cell. In some embodiments, the cardiomyocytes disclosed herein can be differentiated in vitro from an immature cardiomyocyte or a precursor thereof. In some embodiments, the cardiomyocyte is differentiated in vitro from a precursor selected from the group consisting of an immature cardiomyocyte, a cardiac progenitor cell, and a pluripotent stem cell. In some embodiments, the pluripotent stem cell is selected from the group consisting of an embryonic stem cell and induced pluripotent stem cell. In some embodiments, the cardiomyocyte or the pluripotent stem cell from which the cardiomyocyte is derived is human. In some embodiments, the cardiomyocyte is human.

In some embodiments, the cardiomyocyte is not genetically modified. In some embodiments, the cardiomyocyte obtains the features it shares in common with native cardiomyocytes in the absence of a genetic modification of cells. In some embodiments, the cardiomyocyte is genetically modified.

In some aspects, the disclosure provides a cell line comprising a cardiomyocyte described herein. In some embodiments, the cardiomyocytes can be frozen, thawed, and passaged. The cardiomyocytes may be passaged at least 5 times without significant morphological changes.

Aspects of the disclosure relate to isolated populations of cardiomyocytes produced according to methods described herein. In some embodiments, a population of cardiomyocytes is produced by contacting at least one immature cardiomyocyte with at least one cardiomyocyte maturation factor described herein.

Aspects of the disclosure involve microcapsules comprising isolated populations of cells described herein (e.g., cardiomyocytes). Microcapsules are well known in the art. Suitable examples of microcapsules are described in the literature (e.g., Orive et al., “Application of cell encapsulation for controlled delivery of biological therapeutics”, Advanced Drug Delivery Reviews (2013), dx.doi.org/10.1016/j.addr.2013.07.009; Hernandez et al., “Microcapsules and microcarriers for in situ cell delivery”, Advanced Drug Delivery Reviews 2010; 62:711-730; Murua et al., “Cell microencapsulation technology: Towards clinical application”, Journal of Controlled Release 2008; 132:76-83; and Zanin et al., “The development of encapsulated cell technologies as therapies for neurological and sensory diseases”, Journal of Controlled Release 2012; 160:3-13). Microcapsules can be formulated in a variety of ways. Exemplary microcapsules comprise an alginate core surrounded by a polycation layer covered by an outer alginate membrane. The polycation membrane forms a semipermeable membrane, which imparts stability and biocompatibility. Examples of polycations include, without limitation, poly-L-lysine, poly-L-ornithine, chitosan, lactose modified chitosan, and photopolymerized biomaterials. In some embodiments, the alginate core is modified, for example, to produce a scaffold comprising an alginate core having covalently conjugated oligopeptides with an RGD sequence (arginine, glycine, aspartic acid). In some embodiments, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some embodiments, microcapsules are composed of enzymatically modified alginates using epimerases. In some embodiments, microcapsules comprise covalent links between adjacent layers of the microcapsule membrane. In some embodiment, the microcapsule comprises a subsieve-size capsule comprising alginate coupled with phenol moieties. In some embodiments, the microcapsule comprises a scaffold comprising alginate-agarose. In some embodiments, the cardiomyocyte is modified with PEG before being encapsulated within alginate. In some embodiments, the isolated populations of cells, e.g., cardiomyocytes are encapsulated in photoreactive liposomes and alginate. It should be appreciated that the alginate employed in the microcapsules can be replaced with other suitable biomaterials, including, without limitation, PEG, chitosan, PES hollow fibers, collagen, hyaluronic acid, dextran with RGD, EHD and PEGDA, PMBV and PVA, PGSAS, agarose, agarose with gelatin, PLGA, and multilayer embodiments of these.

In some embodiments, compositions comprising populations of cardiomyocytes produced according to the methods described herein can also be used as the functional component in a mechanical device. For example, a device may contain a population of cardiomyocytes (e.g., produced from populations of immature cardiomyocytes or precursors thereof) behind a semipermeable membrane that prevents passage of the cell population, retaining them in the device. Other examples of devices include those contemplated for either implantation into a cardiac patient, or for extracorporeal therapy.

Aspects of the disclosure involve assays comprising isolated populations of cardiomyocytes described herein (e.g., mature cardiomyocytes). In some embodiments, the assays can be used for identifying one or more candidate agents which promote or inhibit a mature cardiomyocyte fate. In some embodiments, the assays can be used for identifying one or more candidate agents which promote the differentiation of at least one immature cardiomyocyte or a precursor thereof into cardiomyocytes. In some embodiments, the assays can be used for identifying one or more candidate agents which promote the shift from immature cardiomyocytes in a senescent state to mature cardiomyocytes in a quiescent state.

The disclosure contemplates methods in which cardiomyocytes are generated according to the methods described herein from iPS cells derived from cells extracted or isolated from individuals suffering from a disease (e.g., heart failure, or a cardiac-related disorder), and those cardiomyocytes are compared to normal cardiomyocytes from healthy individuals not having the disease to identify differences between the cardiomyocytes and normal cardiomyocytes which could be useful as markers for disease (e.g., epigenetic and/or genetic). In some embodiments, cardiomyocytes are obtained from an individual suffering from heart failure and compared to normal cardiomyocytes, and then the cardiomyocytes are reprogrammed to iPS cells and the iPS cells are analyzed for genetic and/or epigenetic markers which are present in the cardiomyocytes obtained from the individual suffering from heart failure but not present in the normal cardiomyocytes, to identify markers (e.g., pre-heart failure). In some embodiments, the iPS cells and/or cardiomyocytes derived from patients are used to screen for agents (e.g., agents which are able to modulate genes contributing to a heart failure phenotype).

### Confirmation of the Presence and the Identification of Cardiomyocytes

One can use any means common to one of ordinary skill in the art to confirm the presence of a quiescent cardiomyocyte as compared to the presence of a senescent cardiomyocyte. One can use any means common to one of ordinary skill in the art to confirm the presence of a cardiomyocyte, e.g. a mature cardiomyocyte produced by the differentiation of at least one immature cardiomyocyte or precursor thereof by exposure to at least one cardiomyocyte maturation factor as described herein.

In some embodiments, the presence of quiescent markers can be done by detecting the presence or absence of one or more markers indicative of a quiescent state. In some embodiments, the method can include detecting the positive expression of quiescence markers p16, p53, and/or p130. In some embodiments, the method can include detecting the negative expression of proliferative markers Ki67, cyclin C1, and/or E2F.

In some embodiments, the presence of cardiomyocyte markers, e.g. chemically induced cardiomyocytes, can be done by detecting the presence or absence of one or more markers indicative of an endogenous cardiomyocyte. In some embodiments, the method can include detecting the positive expression (e.g. the presence) of a marker for cardiomyocytes. In some embodiments the method can include detecting the positive expression of one or more sarcomeric proteins (e.g., cardiac troponin T (TNNT2), cardiac troponin I (TNNI3), myosin heavy chain protein 6 (MYH6) and myosin heavy chain protein 7 (MYH7)). In some embodiments the method can include detecting the positive expression of one or more ion channels (e.g., KCNJ2, HCN4, SCN5a, RYR2, CACNA1c, and SERCA2a (ATP2a2)). In some embodiments the method can include detecting the positive expression of brain natriuretic peptide (BNP). In some embodiments the method can include detecting the positive expression of one or more transcription factors (e.g., REST). In some embodiments the method can include detecting the positive expression of one or more regulators of the oxidative phosphorylation pathway (e.g., PGC1-alpha (PPARGC1a)). In some embodiments, the marker can be detected using a reagent, e.g., a reagent for the detection of TNNT2, TNNI3 and/or KCNJ2. In one aspect, cardiomyocytes herein express TNNI3 and KCNJ2, and do not express significant levels of other markers which would be indicative of immature cardiomyocytes. In one aspect, cardiomyocytes herein express TNNT2 and KCNJ2, and do not express significant levels of other markers which would be indicative of immature cardiomyocytes. Cardiomyocytes can also be characterized by the down-regulation of specific markers. For example, cardiomyocytes may be characterized by a statistically significant down-regulation of HCN4.

A reagent for a marker can be, for example, an antibody against the marker or primers for a RT-PCR or PCR reaction, e.g., a semi-quantitative or quantitative RT-PCR or PCR reaction. Such markers can be used to evaluate whether a cardiomyocyte has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one immature cardiomyocyte from a senescent state to a quiescent state can be monitored by determining the expression of markers characteristic of quiescent cardiomyocytes. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of quiescent cardiomyocytes as well as the lack of significant expression of markers characteristic of senescent cardiomyocytes from which it was derived is determined.

The progression of at least one immature cardiomyocyte or precursor thereof to a cardiomyocyte can be monitored by determining the expression of markers characteristic of mature cardiomyocytes. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of mature cardiomyocytes as well as the lack of significant expression of markers characteristic of immature cardiomyocytes or precursors thereof from which it was derived is determined.

As described in connection with monitoring the production of a cardiomyocyte (e.g., a mature cardiomyocyte) from an immature cardiomyocyte, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of technique such as quantitative-PCR by methods ordinarily known in the art. Additionally, techniques for measuring extracellular marker content, such as ELISA, may be utilized.

It is understood that the present invention is not limited to those markers listed as cardiomyocyte markers herein, and the present invention also encompasses markers such as cell surface markers, antigens, and other gene products including ESTs, RNA (including microRNAs and antisense RNA), DNA (including genes and cDNAs), and portions thereof.

### Enrichment, Isolation and Purification of a Cardiomyocyte

Another aspect of the present invention relates to the isolation of a population of cardiomyocytes (e.g., mature cardiomyocytes) from a heterogeneous population of cells, such as a mixed population of cells comprising mature cardiomyocytes and immature cardiomyocytes or precursors thereof from which the mature cardiomyocyte was derived. In some aspects, a population of quiescent cardiomyocytes are isolated from a heterogenous population of cells, such as a mixed population of cells comprising senescent cardiomyocytes and quiescent cardiomyocytes. A population of cardiomyocytes produced by any of the above-described processes can be enriched, isolated and/or purified by using any cell surface marker present on the cardiomyocyte which is not present on the immature cardiomyocyte or precursor thereof from which it was derived. Such cell surface markers are also referred to as an affinity tag which is specific for a cardiomyocyte (e.g., a mature cardiomyocyte). Examples of affinity tags specific for cardiomyocytes are antibodies, ligands or other binding agents that are specific to a marker molecule, such as a polypeptide, that is present on the cell surface of a cardiomyocyte but which is not substantially present on other cell types (e.g. immature cardiomyocytes). In some processes, an antibody which binds to a cell surface antigen on a cardiomyocyte is used as an affinity tag for the enrichment, isolation or purification of chemically induced (e.g. by contacting with at least one cardiomyocyte maturation factor as described herein) cardiomyocytes produced by the methods described herein. Such antibodies are known and commercially available.

The skilled artisan will readily appreciate the processes for using antibodies for the enrichment, isolation and/or purification of cardiomyocytes. For example, in some embodiments, the reagent, such as an antibody, is incubated with a cell population comprising cardiomyocytes, wherein the cell population has been treated to reduce intercellular and substrate adhesion. The cell population is then washed, centrifuged and resuspended. In some embodiments, if the antibody is not already labeled with a label, the cell suspension is then incubated with a secondary antibody, such as an FITC-conjugated antibody that is capable of binding to the primary antibody. The cardiomyocytes are then washed, centrifuged and resuspended in buffer. The cardiomyocyte suspension is then analyzed and sorted using a fluorescence activated cell sorter (FACS). Antibody-bound, fluorescent reprogrammed cells are collected separately from non-bound, non-fluorescent cells, thereby resulting in the isolation of cardiomyocytes from other cells present in the cell suspension, e.g. immature cardiomyocytes or precursors thereof.

In another embodiment of the processes described herein, the isolated cell composition comprising cardiomyocytes can be further purified by using an alternate affinity-based method or by additional rounds of sorting using the same or different markers that are specific for cardiomyocytes. For example, in some embodiments, FACS sorting is used to first isolate a cardiomyocyte which expresses TNNT2, either alone or with the expression of KCNJ2, or alternatively with a cardiomyocyte marker disclosed herein from cells that do not express one of those markers (e.g. negative cells) in the cell population. In some aspects, TNNI3 and/or MYH7 are also used as markers for FACS sorting, either alone or in combination with TNNT2 and/or KCNJ2. A second FACS sorting, e.g. sorting the positive cells again using FACS to isolate cells that are positive for a different marker than the first sort enriches the cell population for reprogrammed cells.

In an alternative embodiment, FACS sorting is used to separate cells by negatively sorting for a marker that is present on most immature cardiomyocytes but is not present on cardiomyocytes (e.g., mature cardiomyocytes).

In some embodiments of the processes described herein, cardiomyocytes are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above.

In addition to the procedures just described, chemically induced cardiomyocytes may also be isolated by other techniques for cell isolation. Additionally, cardiomyocytes may also be enriched or isolated by methods of serial subculture in growth conditions which promote the selective survival or selective expansion of the cardiomyocytes. Such methods are known by persons of ordinary skill in the art.

Using the methods described herein, enriched, isolated and/or purified populations of cardiomyocytes can be produced in vitro from immature cardiomyocytes or precursors thereof (which were differentiated from pluripotent stem cells by the methods described herein). In some embodiments, preferred enrichment, isolation and/or purification methods relate to the in vitro production of human cardiomyocytes from human immature cardiomyocytes or precursors thereof, which were differentiated from human pluripotent stem cells, or from human induced pluripotent stem (iPS) cells. In such an embodiment, where cardiomyocytes are differentiated from immature cardiomyocytes, which were previously derived from iPS cells, the cardiomyocytes can be autologous to the subject from whom the cells were obtained to generate the iPS cells.

Using the methods described herein, isolated cell populations of cardiomyocytes are enriched in cardiomyocyte (e.g., mature cardiomyocyte) content by at least about 1- to about 1000-fold as compared to a population of cells before the chemical induction of the immature cardiomyocyte or precursor population. In some embodiments the population of cardiomyocytes is induced, enhances, enriched, or increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more as compared to a population of cells before the chemical induction of immature cardiomyocyte or precursor population.

### Compositions Comprising Cardiomyocytes

Some embodiments of the present invention relate to cell compositions, such as cell cultures or cell populations, comprising cardiomyocytes, wherein the cardiomyocytes have been derived from at least one immature cardiomyocyte. In some embodiments, the cell compositions comprise immature cardiomyocytes.

In some embodiments, the cell compositions comprise quiescent cardiomyocytes, wherein the quiescent cardiomyocytes have been derived from at least one senescent cardiomyocyte. In some embodiments, the cell compositions comprise senescent cardiomyocytes.

In accordance with certain embodiments, the chemically induced cardiomyocytes are mammalian cells, and in a preferred embodiment, such cardiomyocytes are human cardiomyocytes. In some embodiments, the immature cardiomyocytes have been derived from pluripotent stem cells (e.g., human pluripotent stem cells).

In some embodiments, the cell compositions comprise mature cardiomyocytes, wherein the mature cardiomyocytes have been derived from at least one immature cardiomyocyte using methods described herein. For example, the cell compositions comprise mature cardiomyocytes obtained from the culturing of immature cardiomyocytes in 2D or 3D culture, wherein the immature cardiomyocytes were contacted with one or more cardiomyocyte maturation factors.

Other embodiments of the present invention relate to compositions, such as an isolated cell population or cell culture, comprising cardiomyocytes produced by the methods as disclosed herein. In such embodiments, the cardiomyocytes comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the cardiomyocyte population. In some embodiments, the composition comprises a population of cardiomyocytes which make up more than about 90% of the total cells in the cell population, for example about at least 95%, or at least 96%, or at least 97%, or at least 98% or at least about 99%, or about at least 100% of the total cells in the cell population are cardiomyocytes.

Certain other embodiments of the present invention relate to compositions, such as an isolated cell population or cell cultures, comprising a combination of cardiomyocytes (e.g., mature cardiomyocytes) and immature cardiomyocytes or precursors thereof from which the cardiomyocytes were derived. In some embodiments, the immature cardiomyocytes from which the cardiomyocytes are derived comprise less than about 25%, less than about 20%, less than about 15%, less than about 10%, less than about 5%, less than about 4%, less than about 3%, less than about 2% or less than about 1% of the total cells in the isolated cell population or culture.

Additional embodiments of the present invention relate to compositions, such as isolated cell populations or cell cultures, produced by the processes described herein and which comprise chemically induced cardiomyocytes as the majority cell type. In some embodiments, the methods and processes described herein produce an isolated cell culture and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% cardiomyocytes.

In another embodiment, isolated cell populations or compositions of cells (or cell cultures) comprise human cardiomyocytes. In other embodiments, the methods and processes as described herein can produce isolated cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% cardiomyocytes. In preferred embodiments, isolated cell populations can comprise human cardiomyocytes. In some embodiments, the percentage of cardiomyocytes in the cell cultures or populations is calculated without regard to the feeder cells remaining in the culture.

Still other embodiments of the present invention relate to compositions, such as isolated cell populations or cell cultures, comprising mixtures of cardiomyocytes and immature cardiomyocytes or precursors thereof from which they were differentiated or matured from. For example, cell cultures or cell populations comprising at least about 5 cardiomyocytes for about every 95 immature cardiomyocytes or precursors thereof can be produced. In other embodiments, cell cultures or cell populations comprising at least about 95 cardiomyocytes for about every 5 immature cardiomyocytes or precursors thereof can be produced. Additionally, cell cultures or cell populations comprising other ratios of cardiomyocytes to immature cardiomyocytes or precursors thereof are contemplated. For example, compositions comprising at least about 1 cardiomyocyte for about every 1,000,000, or at least 100,000 cells, or at least 10,000 cells, or at least 1000 cells or 500, or at least 250 or at least 100 or at least 10 immature cardiomyocytes or precursors thereof can be produced.

Further embodiments of the present invention relate to compositions, such as cell cultures or cell populations, comprising human cells, including human cardiomyocytes, which displays at least one characteristic of an endogenous cardiomyocyte.

In preferred embodiments of the present invention, cell cultures and/or cell populations of cardiomyocytes comprise human cardiomyocytes that are non-recombinant cells. In such embodiments, the cell cultures and/or cell populations are devoid of or substantially free of recombinant human cardiomyocytes.

### Cardiomyocyte Maturation Factors

Aspects of the disclosure involve contacting immature cardiomyocytes or precursors thereof with cardiomyocyte maturation factors, for example, to induce the maturation of the immature cardiomyocytes or differentiation of the precursors thereof into cardiomyocytes (e.g., mature cardiomyocytes). The term “cardiomyocyte maturation factor” refers to an agent that promotes or contributes to the conversion of at least one immature cardiomyocyte or a precursor thereof to a cardiomyocyte. In some embodiments, the cardiomyocyte maturation factor induces the differentiation of pluripotent cells (e.g., iPSCs or hESCs) into immature cardiomyocytes, e.g., in accordance with a method described herein. In some embodiments, the cardiomyocyte maturation factor induces the maturation of immature cardiomyocytes into cardiomyocytes, e.g., in accordance with a method described herein. In some embodiments, a cardiomyocyte maturation factor induces a senescent cardiomyocyte to transition to a quiescent cardiomyocyte.

Generally, at least one cardiomyocyte maturation factor described herein can be used alone, or in combination with other cardiomyocyte maturation factors, to generate cardiomyocytes according to the methods as disclosed herein. In some embodiments, at least two, at least three, at least four, at least five, at least six, at least seven, at least eight, at least nine, or at least ten cardiomyocyte maturation factors described herein are used in the methods of generating cardiomyocytes (e.g., mature cardiomyocytes).

In some embodiments, a cardiomyocyte maturation factor comprises a modulator (e.g., inhibitor) of the phosphoinositide 3-kinase (PI3K)/Akt/mTOR pathway. In some embodiments, a cardiomyocyte maturation factor comprises an inhibitor of the mTOR pathway. In some embodiments, a cardiomyocyte maturation factor comprises an inhibitor of PI3K and/or Akt. In some embodiments, a cardiomyocyte maturation factor comprises a small molecule, nucleic acid, amino acid, metabolite, polypeptide, antibody and antibody-like molecules, aptamers, macrocycles, or other molecules. In some embodiments, a cardiomyocyte maturation factor is selected from the group consisting of Torin1, Torin2, rapamycin, everolimus, and temsirolimus. In some embodiments, a cardiomyocyte maturation factor is Torin1. In some embodiments, a cardiomyocyte maturation factor is Torin2. In some embodiments, a cardiomyocyte maturation factor is rapamycin. In some embodiments, a cardiomyocyte maturation factor is everolimus. In some embodiments, a cardiomyocyte maturation factor is temsirolimus.

In some embodiments, a cardiomyocyte maturation factor comprises a modulator of senescent cells. For example, a modulator of senescent cells may be a senolytic. In some embodiments, a cardiomyocyte maturation factor reduces, and in certain aspects eliminates, senescent cells. In some embodiments, a cardiomyocyte maturation factor is selected from the group consisting of fisetin, luteolin, curcumin, geldanamycin, tanespimycin, alvespimyycin, piperlongumine, FOXO4-related peptide, nutlin-3a, ouabain, proscillaridin A, digoxin, quercetin, dasatinib, navitoclax, and combinations thereof. In certain embodiments, a cardiomyocyte maturation factor is quercetin. In some aspects, quercetin increases expression of p53 and/or Kir2.1.

In some embodiments, a cardiomyocyte maturation factor comprises a modulator (e.g., upregulator) of the cell cycle regulator p53. In some embodiments, a cardiomyocyte maturation factor comprises an upregulator or activator of p53. In some embodiments, a cardiomyocyte maturation factor comprises a small molecule, nucleic acid, amino acid, metabolite, polypeptide, antibody and antibody-like molecules, aptamers, macrocycles, or other molecules. In some embodiments, a cardiomyocyte maturation factor is an MDM2 inhibitor. In some embodiments, a cardiomyocyte maturation factor is selected from the group consisting of RG7112, idasanutlin, AMG-232, APG-115, BI-907828, CGM097, siremadlin, milademetan, nutlin-3a, and combinations thereof. In some embodiments, a cardiomyocyte maturation factor is an MDM2 inhibitor (e.g., nutlin-3a). In some embodiments, a cardiomyocyte maturation actor is a senolytic (e.g., quercetin).

In some embodiments, a cardiomyocyte maturation factor is selected from the group consisting of Torin1, nutlin-3a, and quercetin. In some embodiments, a cardiomyocyte maturation factor is Torin1. In some embodiments, a cardiomyocyte maturation factor in nutlin-3a. In some embodiments, a cardiomyocyte maturation factor is quercetin. In some embodiments, p53 is upregulated (e.g., synergistically) by administering one or more of nutlin-3a, quercetin, and Torin1. In some embodiments, a cardiomyocyte maturation factor is not Torin1. In some embodiments, a cardiomyocyte maturation factor is not an mTOR inhibitor.

### Compositions and Kits

Described herein are compositions which comprise a cardiomyocyte described herein (e.g., a mature and/or quiescent cardiomyocyte). In some embodiments, the composition also includes a maturation factor described herein and/or cell culture media. Described herein are also compositions comprising the compounds described herein (e.g., cell culture media comprising one or more of the compounds described herein).

Also described herein are kits for practicing methods disclosed herein and for making cardiomyocytes (e.g., mature and/or quiescent cardiomyocytes) disclosed herein. Also described herein are kits for treating chronic heart failure and reducing the incidence of ventricular arrhythmias. In one aspect, a kit includes at least one immature and/or senescent cardiomyocyte or precursor thereof and at least one maturation factor as described herein, and optionally, the kit can further comprise instructions for converting at least one immature cardiomyocyte or precursor thereof to a population of mature cardiomyocytes using a method described herein (e.g., using 2D or 3D culture). In some embodiments, the kit comprises at least two maturation factors. In some embodiments, the kit comprises at least three maturation factors. In some embodiments, the kit comprises any combination of maturation factors.

In some embodiment, the compound in the kit can be provided in a watertight or gas tight container which in some embodiments is substantially free of other components of the kit. The compound can be supplied in more than one container, e.g., it can be supplied in a container having sufficient reagent for a predetermined number of reactions e.g., 1, 2, 3 or greater number of separate reactions to induce immature and/or senescent cardiomyocytes, or precursors thereof, into mature and/or quiescent cardiomyocytes. A maturation factor can be provided in any form, e.g., liquid, dried or lyophilized form. It is preferred that a compound(s) (e.g., maturation factor) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises informational material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a maturation factor) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit can include one or more containers for the composition containing at least one maturation factor as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for cardiomyocytes, e.g., for a marker described herein, e.g., a reagent for the detection of mature cardiomyocytes. Or in some embodiments, the kit can also comprise reagents for the detection of negative markers of cardiomyocytes for the purposes of negative selection of mature cardiomyocytes or for identification of cells which do not express these negative markers (e.g., cardiomyocytes). The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit can include cardiomyocytes, e.g., mature and/or quiescent cardiomyocytes derived from the same type of immature and/or senescent cardiomyocyte or precursor thereof, for example for the use as a positive cell type control.

### Methods of Administering a Cell

In one embodiment, the cells described herein, e.g. a population of mature cardiomyocytes are transplantable, e.g., a population of cardiomyocytes can be administered to a subject. In some embodiments, the cells described herein, e.g. a population of mature, quiescent cardiomyocytes are transplantable, e.g., a population of cardiomyocytes can be administered to a subject. In some embodiments, the subject who is administered a population of cardiomyocytes is the same subject from whom a pluripotent stem cell used to differentiate into a cardiomyocyte was obtained (e.g. for autologous cell therapy). In some embodiments, the subject is a different subject. In some embodiments, a subject is suffering from chronic heart failure, or is a normal subject. For example, the cells for transplantation (e.g. a composition comprising a population of cardiomyocytes) can be a form suitable for transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of cardiomyocytes can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

### Pharmaceutical Compositions

For administration to a subject, a cell population produced by the methods as disclosed herein, e.g. a population of cardiomyocytes (produced by contacting at least one immature cardiomyocyte with at least one maturation factor (e.g., any one, two, three, or more maturation factors as described herein) can be administered to a subject, for example in pharmaceutically acceptable compositions. These pharmaceutically acceptable compositions comprise a therapeutically-effective amount of a population of mature cardiomyocytes as described above, formulated together with one or more pharmaceutically acceptable carriers (additives) and/or diluents. In some aspects, the pharmaceutically acceptable compositions comprise a therapeutically-effective amount of a population of mature, quiescent cardiomyocytes as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., mature cardiomyocytes, or composition comprising mature cardiomyocytes of the present invention which is effective for producing some desired therapeutic effect in at least a sub-population of cells in an animal at a reasonable benefit/risk ratio applicable to any medical treatment. For example, an amount of a population of mature cardiomyocytes administered to a subject that is sufficient to produce a statistically significant, measurable change in at least one symptom of chronic heart failure, such as systolic heart function or incidence of ventricular arrhythmias, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

By “treatment”, “prevention” or “amelioration” of a disease or disorder is meant delaying or preventing the onset of such a disease or disorder, reversing, alleviating, ameliorating, inhibiting, slowing down or stopping the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the term “administer” refers to the placement of a composition into a subject by a method or route which results in at least partial localization of the composition at a desired site such that the desired effect is produced. Routes of administration suitable for the methods of the invention include both local and systemic administration. Generally, local administration results in more of the administered cardiomyocytes being delivered to a specific location as compared to the entire body of the subject, whereas, systemic administration results in delivery of the cardiomyocytes to essentially the entire body of the subject.

In the context of administering a compound treated cell, the term “administering” also include transplantation of such a cell in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantation between members of different species).

Mature cardiomyocytes or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, ingestion, or topical application. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection. In other preferred embodiments, the compositions are administered via a cell patch. In some embodiments, the compositions are administered via a three-dimensional structure (e.g., a matrix or scaffold). In some embodiments, the compositions are administered via a micro-tissue.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with decreased systolic heart function or ventricular arrhythmias. In addition, the methods and compositions described herein can be used to treat domesticated animals and/or pets.

A subject can be one who has been previously diagnosed with or identified as suffering from or having a disorder characterized with decreased systolic heart function or ventricular arrhythmias. A subject may be someone who has been previously diagnosed with or identified as having heart failure (e.g., chronic heart failure). In some aspects, a subject may be someone who has been previously diagnosed with or identified as having a cardiac-related disease or disorder. In some aspects, a subject may be someone who has been previously diagnosed with congenital heart disease (e.g., systolic heart disease or heart disease as a result of tissue engineering).

In some embodiments of the aspects described herein, the method further comprises diagnosing and/or selecting a subject for decreased systolic heart function or ventricular arrhythmias before treating the subject. In some aspects, the method further comprises diagnosing and/or selecting a subject for a cardiac-related disease or disorder before treating the subject. In some aspects, the method further comprises diagnosing and/or selecting a subject for congenital heart disease before treating the subject.

A cardiomyocyte composition described herein can be administered in combination with a mechanical support device (e.g., ventricular assist devices (VADs) or extracorporeal membrane oxygenation (ECMO) systems used to support ventricular recovery), or in combination with cardiac catheterization procedures to revascularize the heart (e.g., stent placement or balloon angioplasty of coronary arteries, or surgical bypass grafting). A cardiomyocyte composition described herein can be co-administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

The composition comprising cardiomyocytes and/or a pharmaceutically active agent can be administrated to the subject in the same pharmaceutical composition or in different pharmaceutical compositions (at the same time or at different times). When administrated at different times, the composition comprising cardiomyocytes and/or the pharmaceutically active agent can be administered within 5 minutes, 10 minutes, 20 minutes, 60 minutes, 2 hours, 3 hours, 4, hours, 8 hours, 12 hours, 24 hours of administration of the other. When the composition comprising cardiomyocytes and/or the pharmaceutically active agent are administered in different pharmaceutical compositions, routes of administration can be different. In some embodiments, a subject is administered a composition comprising cardiomyocytes. In other embodiments, a subject is administered a composition comprising a pharmaceutically active agent. In another embodiment, a subject is administered a composition comprising a population of cardiomyocytes mixed with a pharmaceutically active agent. In another embodiment, a subject is administered a composition comprising a population of cardiomyocytes and a composition comprising a pharmaceutically active agent, where administration is substantially at the same time, or subsequent to each other.

Toxicity and therapeutic efficacy of administration of compositions comprising a population of cardiomyocytes can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of cardiomyocytes that exhibit large therapeutic indices are preferred.

The amount of a composition comprising a population of cardiomyocytes can be tested using several well-established animal models.

In some embodiments, data obtained from the cell culture assays and in animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of cardiomyocytes can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alterations to a treatment regimen. The dosing schedule can vary from once a week to daily depending on a number of clinical factors. The desired dose can be administered at one time or divided into subdoses, e.g., 2-4 subdoses and administered over a period of time, e.g., at appropriate intervals through the day or other appropriate schedules. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of cardiomyocytes as disclosed herein. In one embodiment of the invention, an isolated population of cardiomyocytes as disclosed herein may be used for the production of a pharmaceutical composition, for the use in transplantation into subjects in need of treatment, e.g. a subject that has, or is at risk of developing a ventricular arrhythmia or decreased systolic heart function (e.g., chronic heart failure). In one embodiment, an isolated population of cardiomyocytes may be genetically modified. In another aspect, the subject may have or be at risk of ventricular arrhythmias or decreased systolic heart function. In some embodiments, an isolated population of cardiomyocytes as disclosed herein may be autologous and/or allogeneic. In some embodiments, the subject is a mammal, and in other embodiments the mammal is a human.

One embodiment of the invention relates to a method of treating chronic heart failure in a subject comprising administering an effective amount of a composition comprising a population of cardiomyocytes as disclosed herein to a subject with chronic heart failure. Other embodiments relate to a method of treating a ventricular arrhythmia in a subject comprising administering an effective amount of a composition comprising a population of cardiomyocytes as disclosed herein to a subject with a ventricular arrhythmia. In a further embodiment, the invention provides a method for treating decreased systolic heart function, comprising administering a composition comprising a population of cardiomyocytes as disclosed herein to a subject with decreased systolic heart function. In another embodiment, the invention provides a method for treating congenital heart disease comprising administering an effective amount of a composition comprising a population of cardiomyocytes as disclosed herein to a subject with congenital heart disease.

In some embodiments, a population of cardiomyocytes as disclosed herein may be administered in any physiologically acceptable excipient, where the cardiomyocytes may find an appropriate site for replication, proliferation, and/or engraftment. In some embodiments, a population of cardiomyocytes as disclosed herein can be introduced by injection, catheter, or the like. In some embodiments, a population of cardiomyocytes as disclosed herein can be frozen at liquid nitrogen temperatures and stored for long periods of time, and is capable of use on thawing. If frozen, a population of cardiomyocytes will usually be stored in a 10% DMSO, 50% FCS, 40% RPMI 1640 medium or other cryoprotective solution. Once thawed, the cells may be expanded by use of growth factors and/or feeder cells associated with culturing cardiomyocytes as disclosed herein.

In some embodiments, a population of cardiomyocytes as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of cardiomyocytes as disclosed herein will be adapted in accordance with the route and device used for administration. In some embodiments, a composition comprising a population of cardiomyocytes can also comprise or be accompanied with one or more other ingredients that facilitate the engraftment or functional mobilization of the cardiomyocytes. Suitable ingredients include matrix proteins that support or promote adhesion of the cardiomyocytes, or complementary cell types. In another embodiment, the composition may comprise resorbable or biodegradable matrix scaffolds.

Gene therapy can be used to either modify a cell to replace a gene product, to facilitate regeneration of tissue, to treat disease, or to improve survival of the cells following implantation into a subject (i.e. prevent rejection).

In one aspect of the present invention, a population of cardiomyocytes as disclosed herein is suitable for administering systemically or to a target anatomical site. A population of cardiomyocytes can be grafted into or nearby a subject's heart, for example, or may be administered systemically, such as, but not limited to, intra-arterial or intravenous administration. In alternative embodiments, a population of cardiomyocytes of the present invention can be administered in various ways as would be appropriate to implant in the cardiac system, including but not limited to parenteral, including intravenous and intraarterial administration, intrathecal administration, intraventricular administration, intraparenchymal, intracranial, intracisternal, intrastriatal, and intranigral administration. Optionally, a population of cardiomyocytes is administered in conjunction with an immunosuppressive agent.

In some embodiments, a population of cardiomyocytes can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of cardiomyocytes can be administered to a subject at the following locations: clinic, clinical office, emergency department, hospital ward, intensive care unit, operating room, catheterization suites, and radiologic suites.

In other embodiments, a population of cardiomyocytes is stored for later implantation/infusion. A population of cardiomyocytes may be divided into more than one aliquot or unit such that part of a population of cardiomyocytes is retained for later application while part is applied immediately to the subject. Moderate to long-term storage of all or part of the cells in a cell bank is also within the scope of this invention, as disclosed in U.S. Patent Publication No. 2003/0054331 and Patent Publication No. WO 03/024215, and is incorporated by reference in their entireties. At the end of processing, the concentrated cells may be loaded into a delivery device, such as a syringe, for placement into the recipient by any means known to one of ordinary skill in the art.

In some embodiments a population of cardiomyocytes can be applied alone or in combination with other cells, tissue, tissue fragments, growth factors such as VEGF and other known angiogenic or arteriogenic growth factors, biologically active or inert compounds, resorbable plastic scaffolds, or other additives intended to enhance the delivery, efficacy, tolerability, or function of the population. In some embodiments, a population of cardiomyocytes may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In another aspect, in some embodiments, a population of cardiomyocytes could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Publication No 2002/0182211, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiomyocytes of the invention.

Pharmaceutical compositions comprising effective amounts of a population of cardiomyocytes are also contemplated by the present invention. These compositions comprise an effective number of cardiomyocytes, optionally, in combination with a pharmaceutically acceptable carrier, additive or excipient. In certain aspects of the present invention, a population of cardiomyocytes is administered to the subject in need of a transplant in sterile saline. In other aspects of the present invention, a population of cardiomyocytes is administered in Hanks Balanced Salt Solution (HBSS) or Isolyte S, pH 7.4. Other approaches may also be used, including the use of serum free cellular media. In one embodiment, a population of cardiomyocytes is administered in plasma or fetal bovine serum, and DMSO. Systemic administration of a population of cardiomyocytes to the subject may be preferred in certain indications, whereas direct administration at the site of or in proximity to the diseased and/or damaged tissue may be preferred in other indications.

In some embodiments, a population of cardiomyocytes can optionally be packaged in a suitable container with written instructions for a desired purpose, such as the reconstitution or thawing (if frozen) of a population of cardiomyocytes prior to administration to a subject.

Methods of Identifying Cardiomyocyte Maturation Factors that Increase the Production of Mature Cardiomyocytes

Described herein is a method of identifying a cardiomyocyte maturation factor or agent that increases the production of cardiomyocytes (e.g., mature cardiomyocytes). In certain examples, a high content and/or high throughput screening method is provided. The method includes exposing at least one immature cardiomyocyte or a precursor thereof to at least one compound (e.g., a library compound or a compound described herein) and determining if the compound increases the production of cardiomyocytes, e.g., mature cardiomyocytes from the at least one immature cardiomyocyte or the precursor thereof. A cell can be identified as a cardiomyocyte (e.g., a mature cardiomyocyte) using one or more of the markers described herein. In some examples, the at least one immature cardiomyocyte or the precursor thereof may be differentiated prior to exposure to the library. In other examples, two or more compounds may be used, either individually or together, in the screening assay. In additional examples, the at least one immature cardiomyocyte or the precursor thereof may be placed in a multi-well plate, and a library of compounds may be screened by placing the various members of the library in different wells of the multi-well plate. Such screening of libraries can rapidly identify compounds that are capable of generating cardiomyocytes, e.g., mature cardiomyocytes, from the at least one immature cardiomyocyte or precursor thereof.

Also described herein is a method of identifying a cardiomyocyte maturation factor or agent that increases the production of cardiomyocytes (e.g., quiescent cardiomyocytes). In certain examples, a high content and/or high throughput screening method is provided. The method includes exposing at least one senescent cardiomyocyte to at least one compound (e.g., a library compound or a compound described herein) and determining if the compound increases the production of cardiomyocytes, e.g., quiescent cardiomyocytes from the at least one senescent cardiomyocyte. A cell can be identified as a cardiomyocyte (e.g., a quiescent cardiomyocyte) using one or more of the markers described herein. In other examples, two or more compounds may be used, either individually or together, in the screening assay. In additional examples, the at least one senescent cardiomyocyte may be placed in a multi-well plate, and a library of compounds may be screened by placing the various members of the library in different wells of the multi-well plate. Such screening of libraries can rapidly identify compounds that are capable of generating cardiomyocytes, e.g., quiescent cardiomyocytes, from the at least one senescent cardiomyocyte.

In some embodiments, the method further comprises isolating a population of the cardiomyocytes, e.g., mature cardiomyocytes (e.g., wherein at least 5%, 10%, 15%, 20%, 25%, 30%, 35%, 50%, 75% or greater are of the subject cell type).

In some embodiments, the method further comprises implanting the cardiomyocytes produced by the methods as disclosed herein into a subject (e.g., a subject having chronic heart failure). In some embodiments, the cardiomyocyte is derived from a stem cell obtained from a subject. In some embodiments, the cardiomyocyte is derived from a stem cell from a donor different than the subject, e.g., a relative of the subject.

In one aspect, the invention features a cardiomyocyte, e.g., a mature cardiomyocyte, made by a method described herein. In another aspect, the invention features a composition comprising a cardiomyocyte made by a method described herein.

In another aspect, the invention features a kit comprising: immature cardiomyocytes or precursors thereof; at least one cardiomyocyte maturation factor described herein; and instructions for using the immature cardiomyocytes or precursors thereof and the at least one cardiomyocyte maturation factor to produce a cardiomyocyte (e.g., a mature cardiomyocyte). In some embodiments, the kit further comprises: a component for the detection of a marker for a mature cardiomyocyte, e.g., for a marker described herein, e.g., a reagent for the detection of a marker of cardiomyocyte maturity, e.g., an antibody against the marker; and a mature cardiomyocyte, e.g., for use as a control.

In one aspect, the invention features a method of facilitating differentiation of immature cardiomyocytes or precursors thereof to cardiomyocytes comprising providing at least one immature cardiomyocyte or precursor thereof, and providing at least one cardiomyocyte maturation factor (e.g., 1, 2, 3, 4, 5, 6, 7, 8, 9, 10 or more cardiomyocyte maturation factors described herein) to mature or differentiate the at least one immature cardiomyocyte or precursor thereof to a cardiomyocyte (e.g., a mature cardiomyocyte), upon exposure of the stem cell to the at least one maturation factor. In some embodiments, the at least one immature cardiomyocyte or precursor thereof is from a mammal. In some embodiments, the at least one immature cardiomyocyte or precursor thereof is from mouse or human. In some embodiments, the at least one immature cardiomyocyte or precursor thereof derived from culturing an embryonic stem cell (e.g., a mammalian embryonic stem cell such as a mouse or human embryonic stem cell). In some embodiments, the at least one immature cardiomyocyte or precursor thereof derived from culturing an induced pluripotent stem cell (e.g., a mammalian iPs cell such as a mouse or human iPs cell).

In some embodiments, a plurality of immature cardiomyocytes or precursors thereof are differentiated or matured into a plurality of mature cardiomyocytes, for example, by contacting the plurality of immature cardiomyocytes or precursors thereof with at least one, at least two, at least three, or more of the cardiomyocyte maturation factors as described herein.

In some embodiments, a plurality of senescent cardiomyocytes is matured into a plurality of quiescent cardiomyocytes, for example, by contacting the plurality of senescent cardiomyocytes with at least one, at least two, at least three, or more of the cardiomyocyte maturation factors as described herein.

In some embodiments, the plurality of immature cardiomyocytes or precursors thereof are exposed to the cardiomyocyte maturation factors, for about 1, 2, 4, 6, 8, 10, 12, 14, 16, or more days. In some embodiments, the plurality of immature cardiomyocyte or precursors thereof are exposed to the cardiomyocyte maturation factors at a concentration of about 25 nM, 50 nM, 100 nM, 150 nM, 200 nM, 250 nM, 400 nM, 500 nM, 600 nM, 700 nM, 800 nM, 1 μM, 2 μM, 3 μM, 4 μM, 5 μM or 10 μM. In some embodiments, the plurality of immature cardiomyocytes or precursors thereof are exposed to the cardiomyocyte maturation factors at a concentration of about 250 nM, 400 nM, 500 nM, 600 nM, 700 nM, or 800 nM. In some embodiments, greater than about 20%, 30%, 40%, 50%, 60%, 70%, 80%, or 90% of the immature cardiomyocytes or precursors thereof are differentiated or matured into the mature cardiomyocytes.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the disclosure. Further, all patents, patent applications, and publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents

### EXAMPLES

### Example 1: Senescence Suppression for Maturation of Stem Cell-Derived Cardiomyocytes

### Background:

Stem cell approaches to treat chronic heart failure will require production of ventricular cardiomyocytes to improve systolic heart function and reduce the incidence of ventricular arrhythmias. However, cardiomyocytes derived from embryonic or induced pluripotent stem cells (ESCs or iPSCs, respectively) remain functionally immature using current differentiation protocols. These immature cardiomyocytes display automaticity or pacemaker-like activity which results in potentially life-threatening ventricular arrhythmias when delivered to adult animal models and also have a less organized sarcomere structure preventing adequate contractile force (1, 2). Successful translation of stem cell-derived therapies for treatment of cardiovascular disease will require developing improved methods for maturation of stem cell-derived cardiomyocytes.

At birth, mammals undergo significant physiologic changes, as the newborn adapts from deriving all oxygen and nutrients from the placenta to deriving oxygen via spontaneous respiration and nutrition via enteral feeding. The underlying molecular mechanisms by which these physiologic changes regulate cardiac phenotype remain unclear. In mice, cardiomyocytes retain the ability to regenerate following myocardial injury in the first few days after birth. (3) However, beyond this period, cardiomyocytes exit the cell cycle and become quiescent. (4) It was recently identified that inhibition of the mechanistic target of rapamycin (mTOR) pathway improves maturation of iPSC-CMs in two-dimensional (2D) culture via induction of cellular quiescence and suppression of cellular senescence (5).

Cellular quiescence is a resting state triggered by nutrient deprivation and is characterized by the ability to re-enter the cell cycle in response to appropriate stimuli (6). In contrast, cellular senescence is a state of irreversible cell cycle arrest associated with an aging or diseased phenotype, characterized by DNA damage, elevated reactive oxygen species levels, and expression of a senescence-associated secretory phenotype (SASP) that can lead to a pro-inflammatory phenotype in nearby cells (7). It was found that iPSC-CMs with a senescent phenotype are less mature, with lower expression of sarcomere or ion channel proteins seen in more mature cardiomyocytes. Because SASP proteins such as interleukin-6 (IL-6) can have detrimental effects on nearby cells due to a paracrine effect, it will be tested whether removal of senescent cells with a senolytic (compound that induces apoptosis of senescent cells) can enhance the maturation of surviving cells.

### Summary of Invention:

It has previously been suggested that inhibition of mTOR signaling with Torin1 during late differentiation both induces cellular quiescence and enhances maturation of cardiomyocytes. In addition, there was a suggestion that quiescence depth may facilitate maturation, and induction of quiescence via p53 upregulation may facilitate iPSC-CM maturation.

Preliminary data suggest that removal of senescent cells from 3D suspension culture may also facilitate maturation of iPSC-CMs. The senolytic, quercetin, is a flavonol found in foods such as apples that can induce apoptosis in senescent tumor cells (8). The preliminary data suggest that treatment of iPSC-CMs with quercetin can enhance iPSC-CM maturation.

### Methods:

The UCSD142i-86-1 cell line was used to generate preliminary data. UCSD142i-86-1 cells were differentiated into cardiomyocytes according to previously published protocols (9) adapted to three-dimensional culture in the laboratory. Two days after onset of beating (around day 9 of differentiation), cardiomyocytes were treated with vehicle (DMSO) or different concentrations of quercetin for different time periods. Cells were harvested for qPCR, western, or flow cytometry at different time points.

### Preliminary Results:

### Quercetin Decreases the Percentage of Live Cells but Increases the Percentage of Cardiomyocytes.

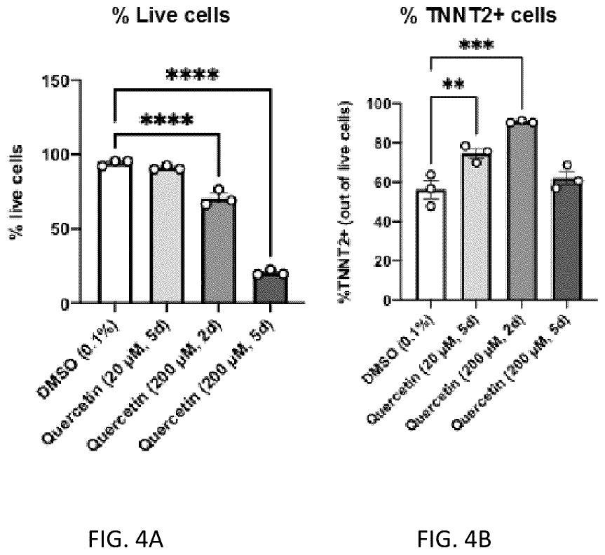

Quercetin treatment (200 μM) decreases the percentage of live cells with 2 or 5 day treatment as quantified by flow cytometry (FIG. 4A). However, 2 day treatment with quercetin at 20 or 200 μM increases the percentage of cardiac troponin T (TNNT2)-positive cardiomyocytes (FIG. 4B), which may suggest that there is purification of cardiomyocytes by cell death of non-cardiomyocytes.

### Quercetin Increases Expression of Kir2.1 and p53 in 3D Culture.

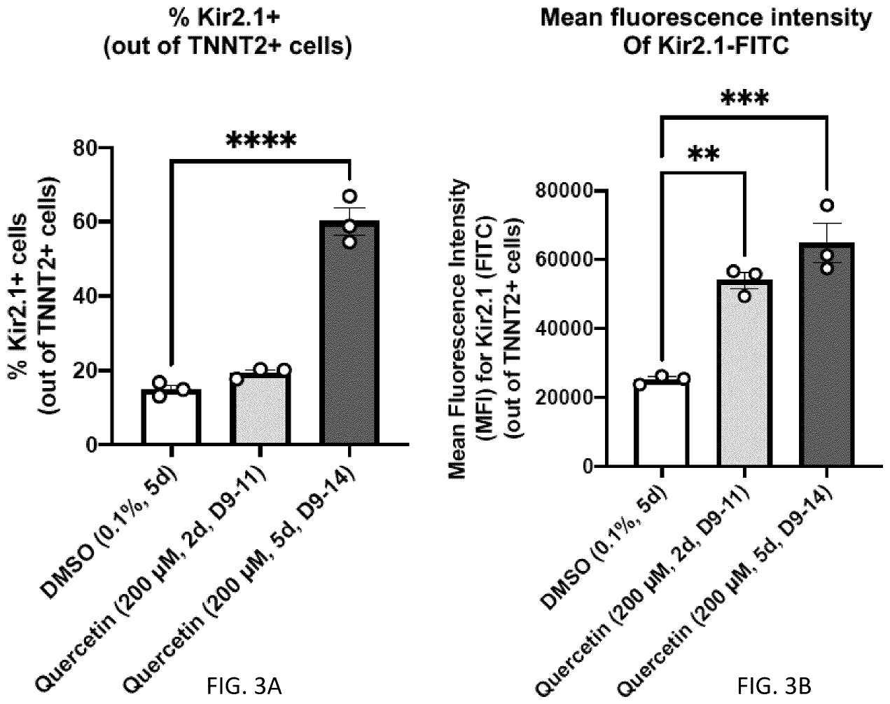

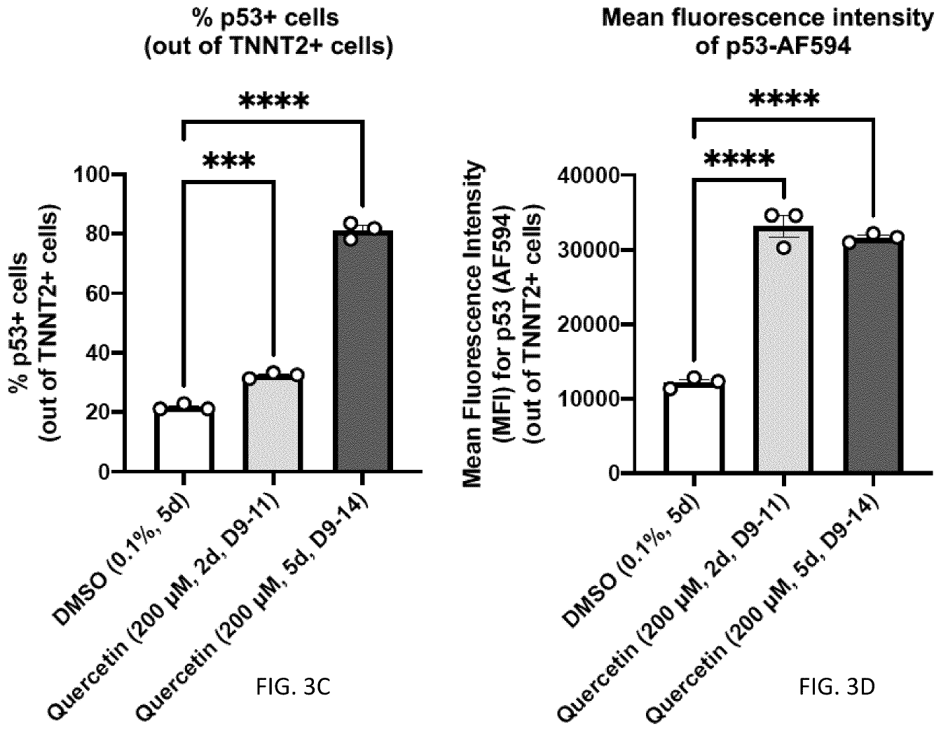

Quercetin treatment of iPSC-CMs increases expression of Kir2.1 (FIGS. 3A-3D), the ion channel largely responsible for maintaining the resting membrane potential via the inward rectifier current (I_{K1}); at lower levels, abnormal membrane depolarization can increase the risk of ventricular arrhythmias (10, 11). In addition, quercetin increases expression of p53, which may support induction of quiescence.

### Quercetin Increases Expression of PPARGC1a.

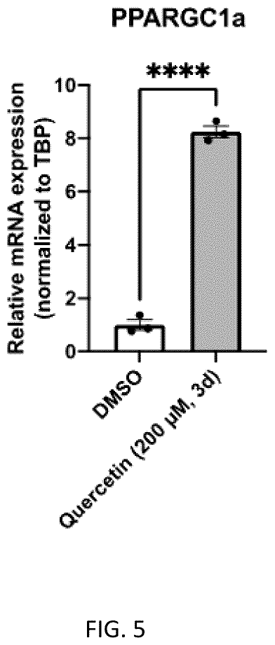

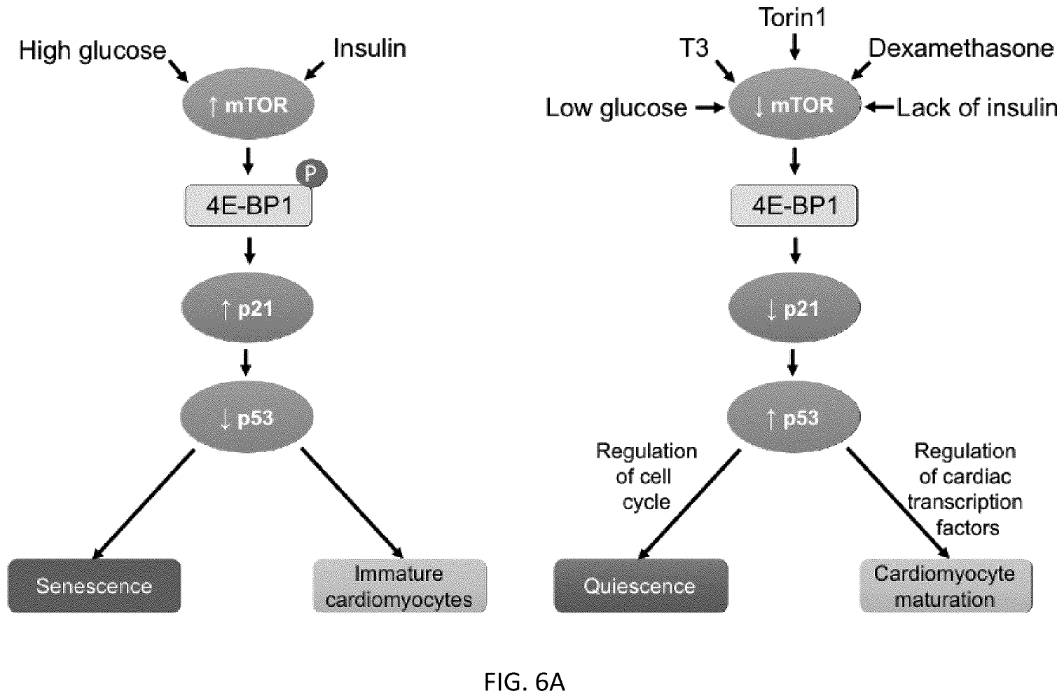

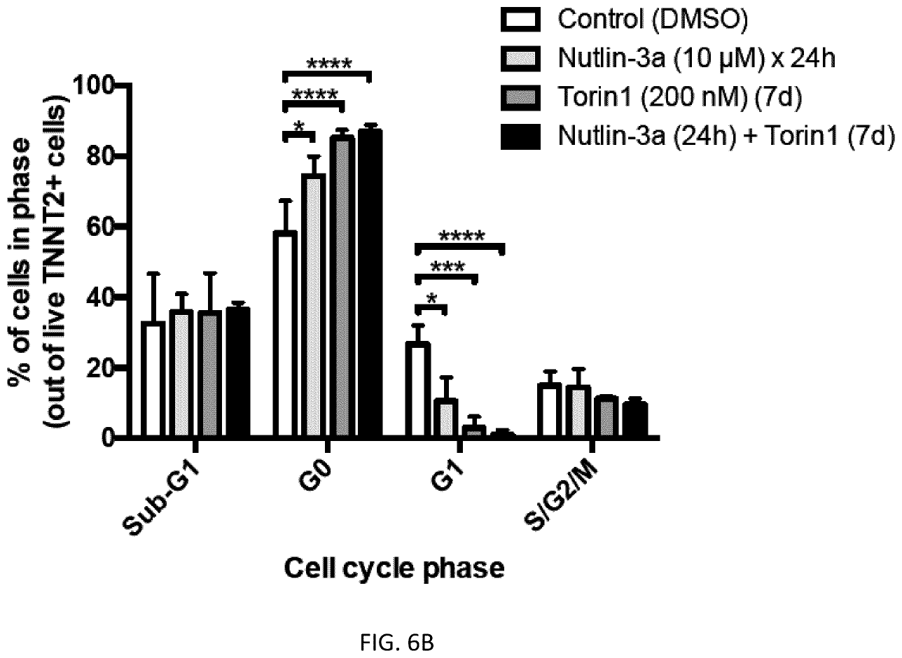

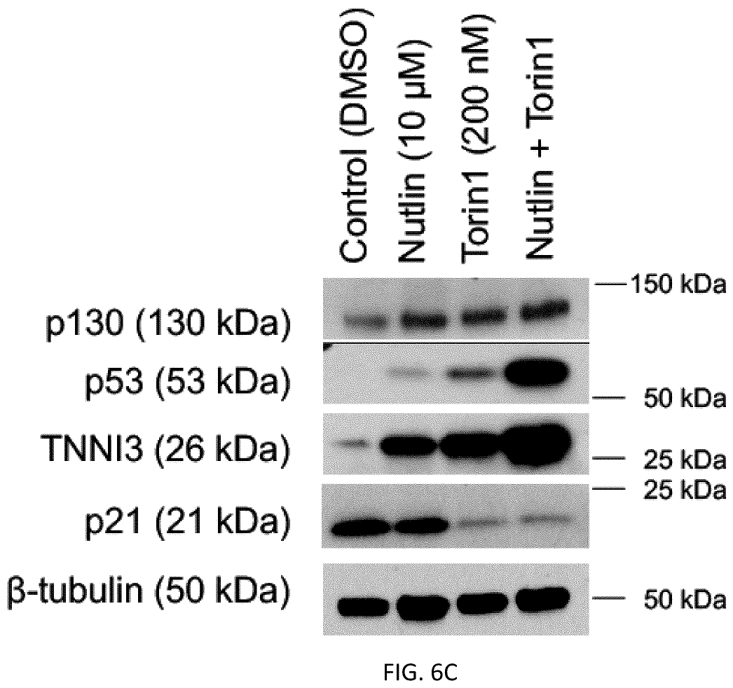

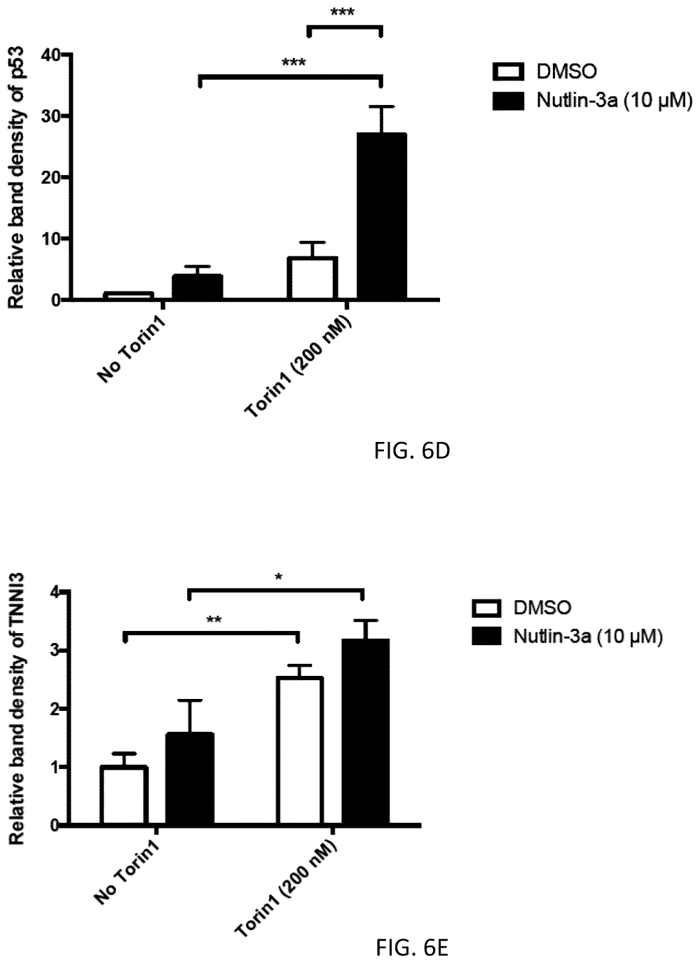

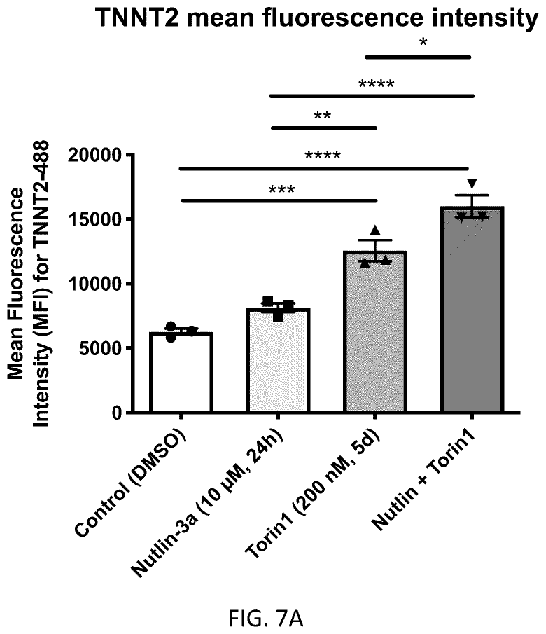

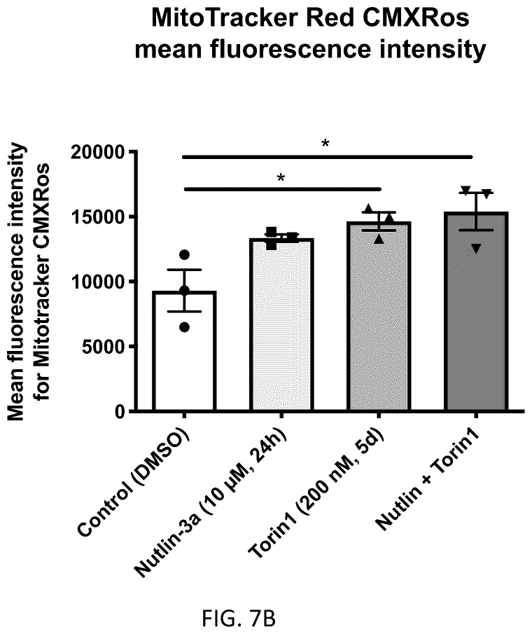

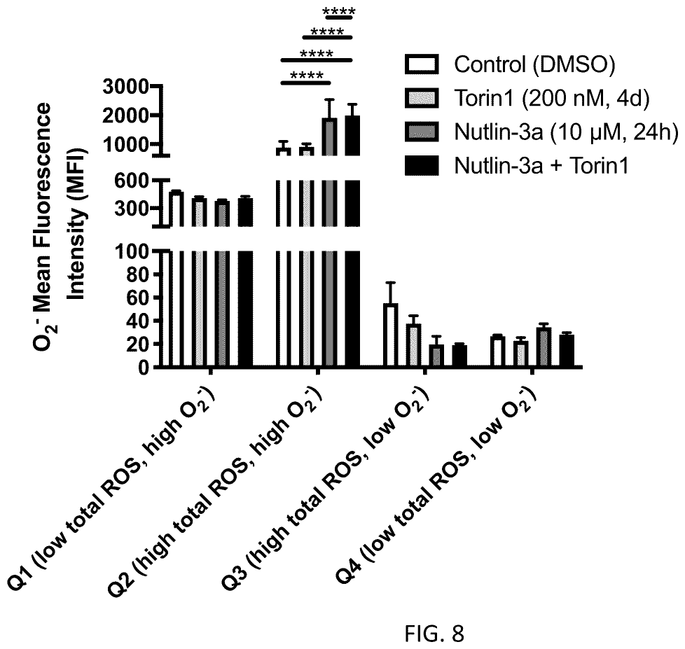

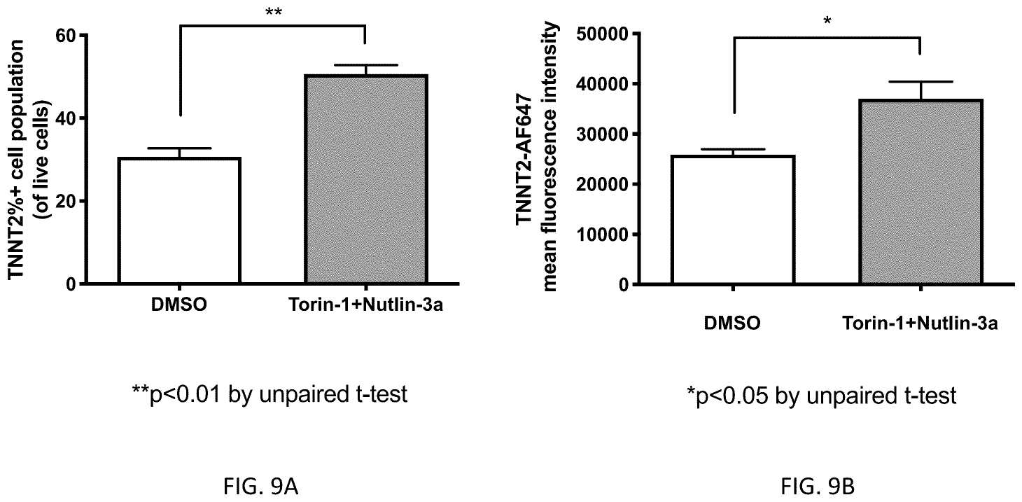

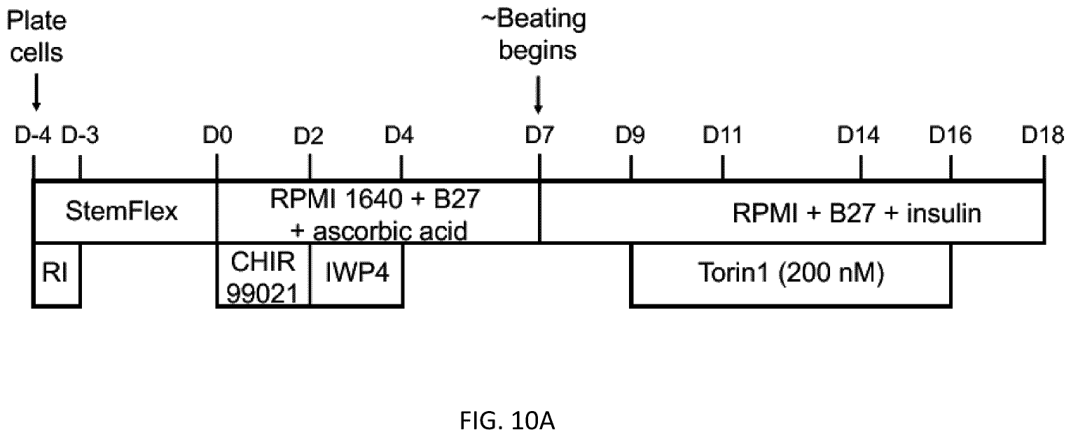

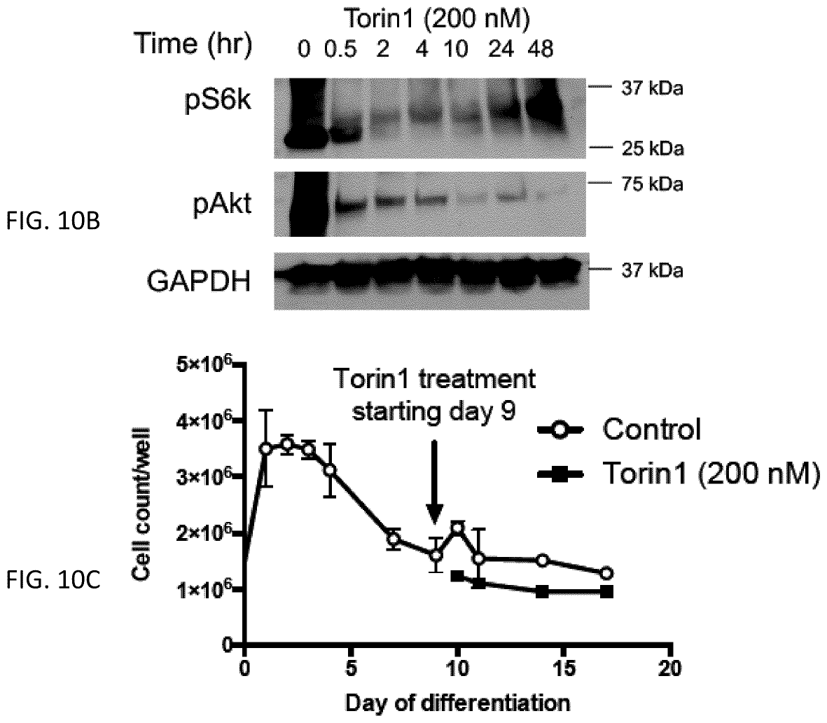

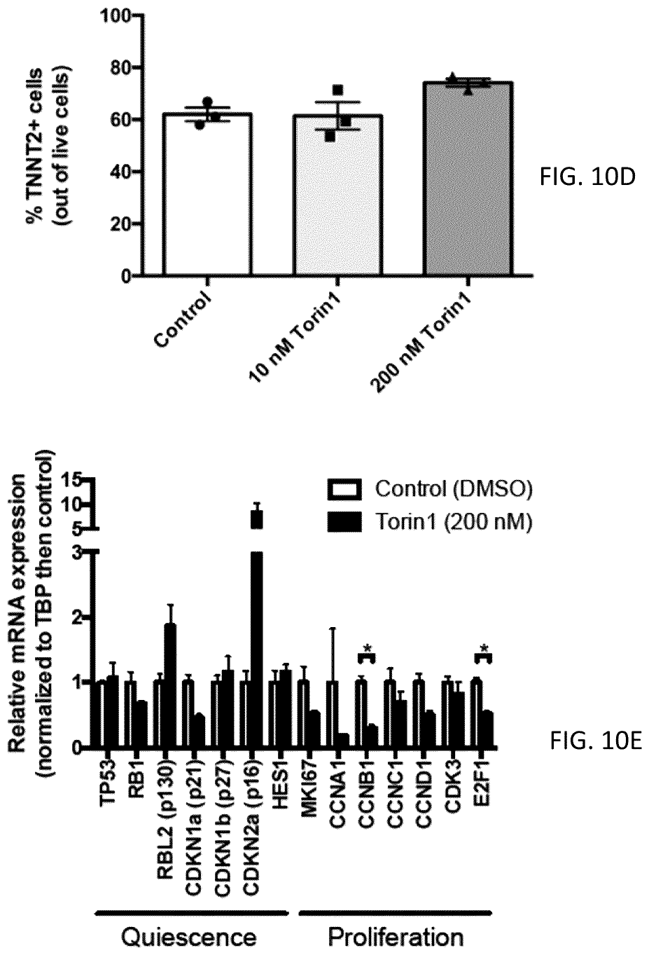

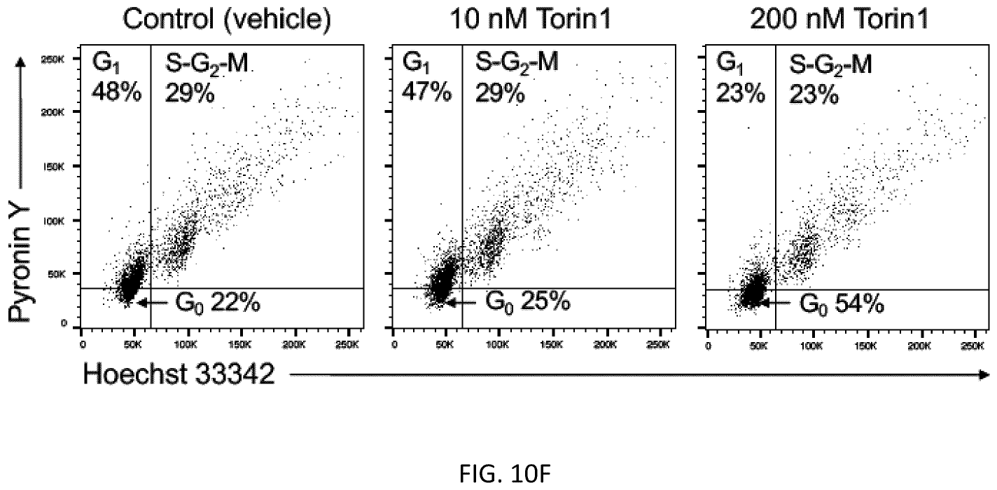

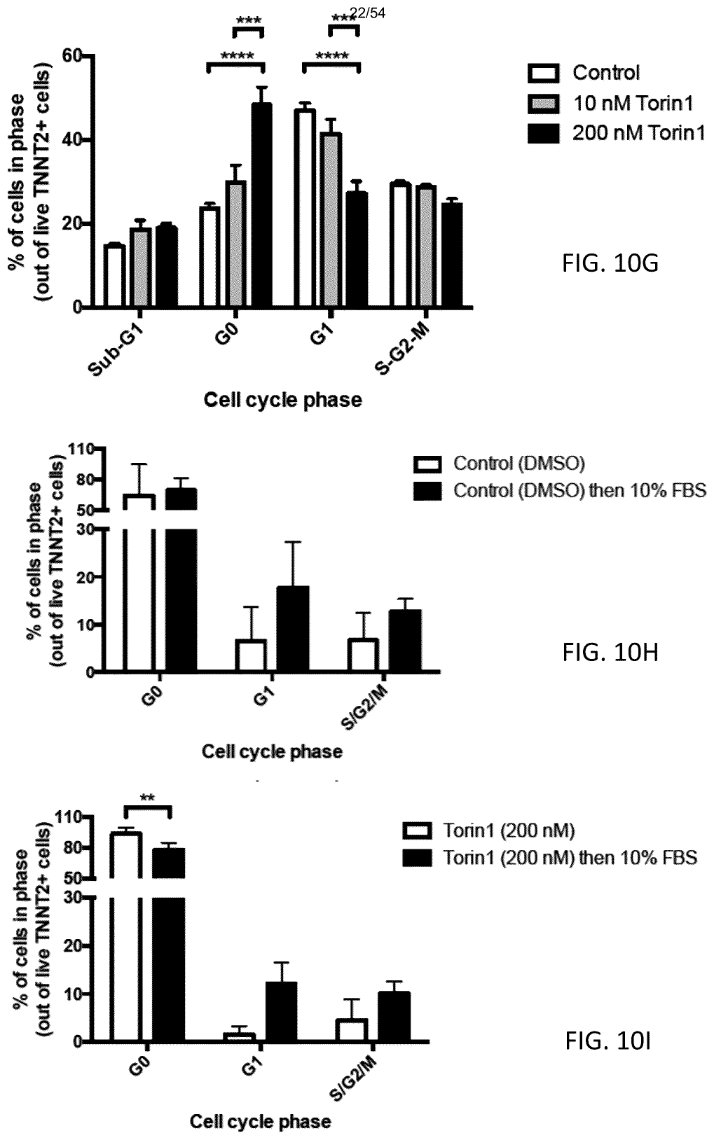

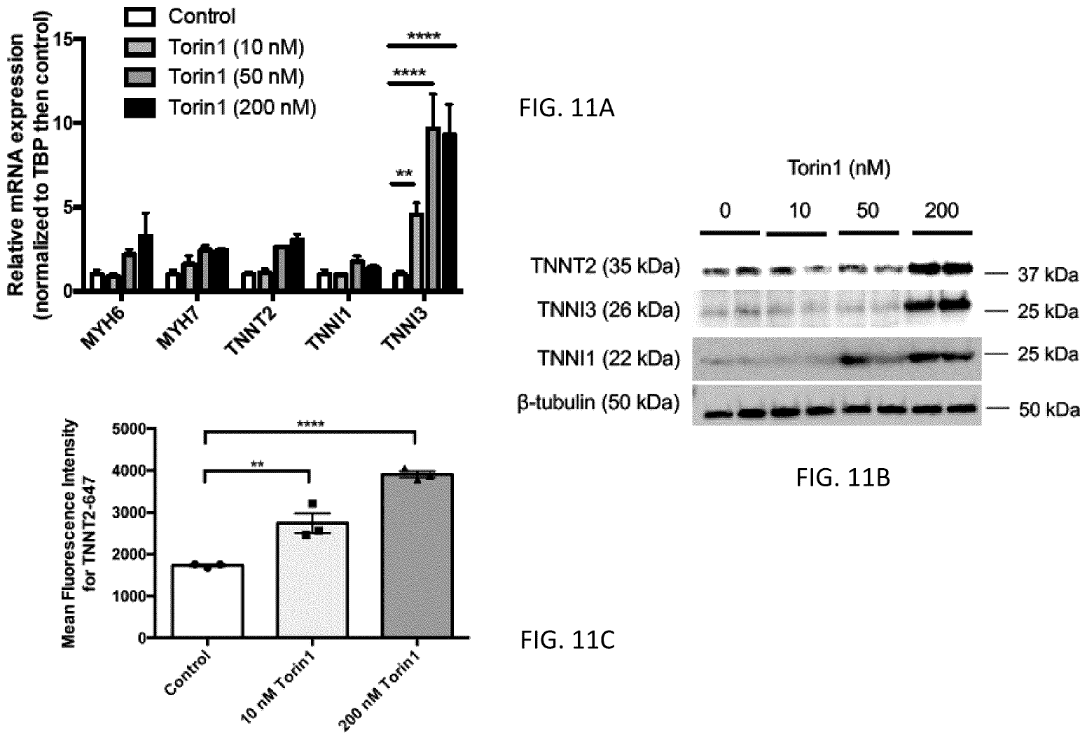

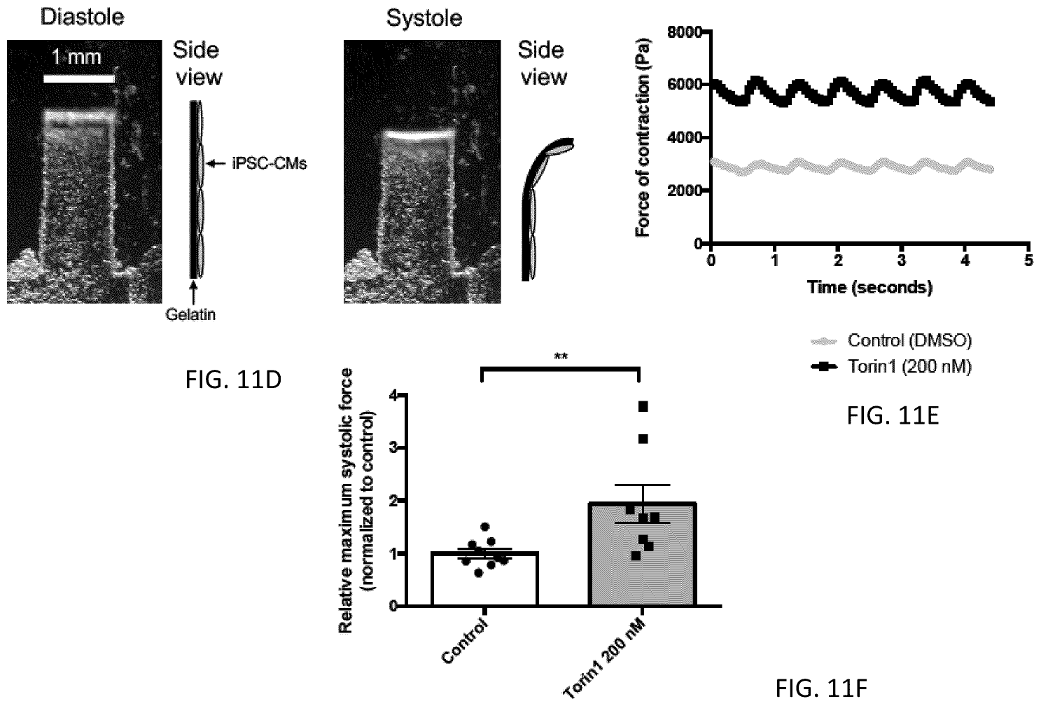

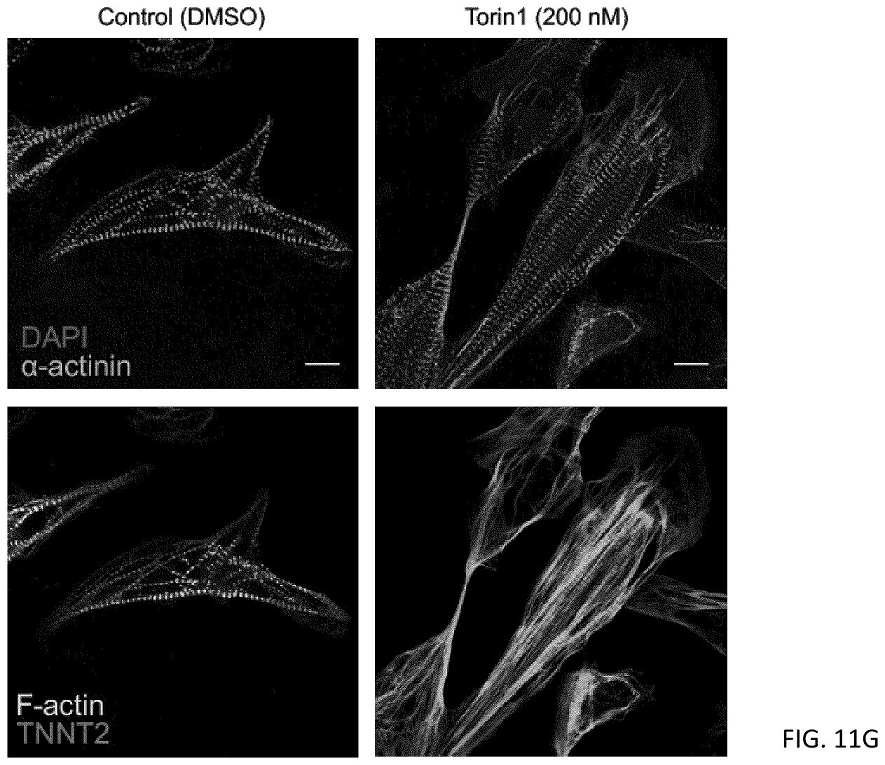

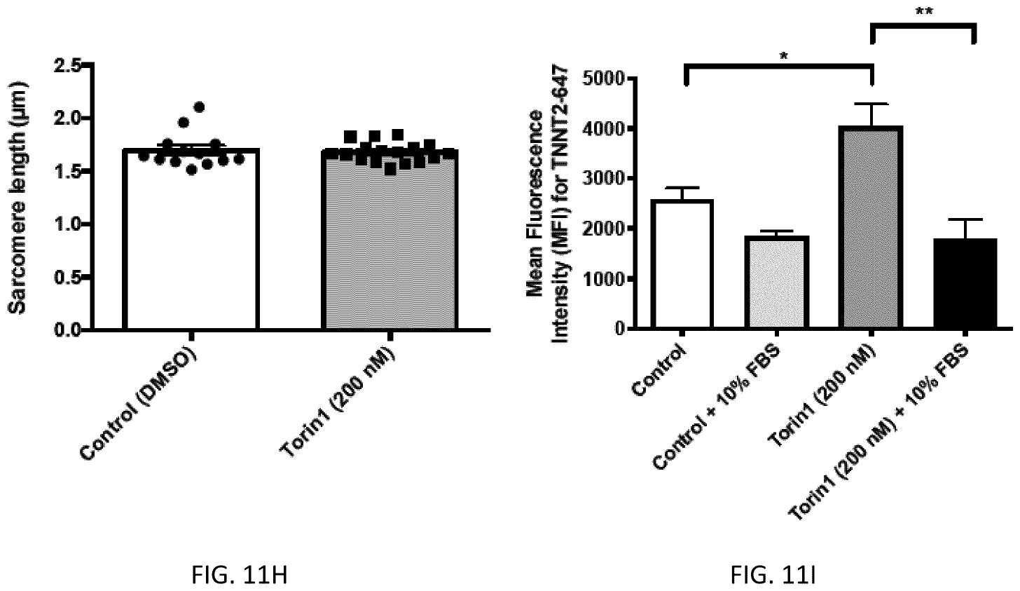

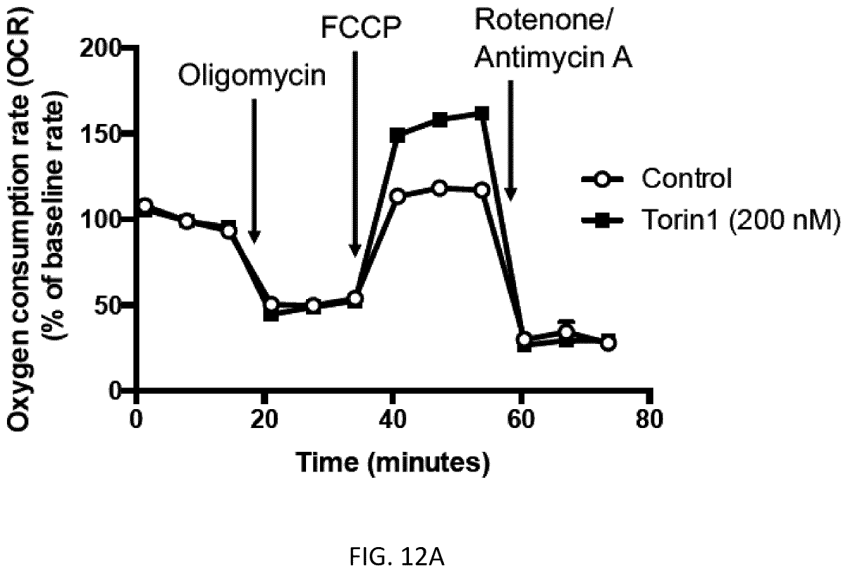

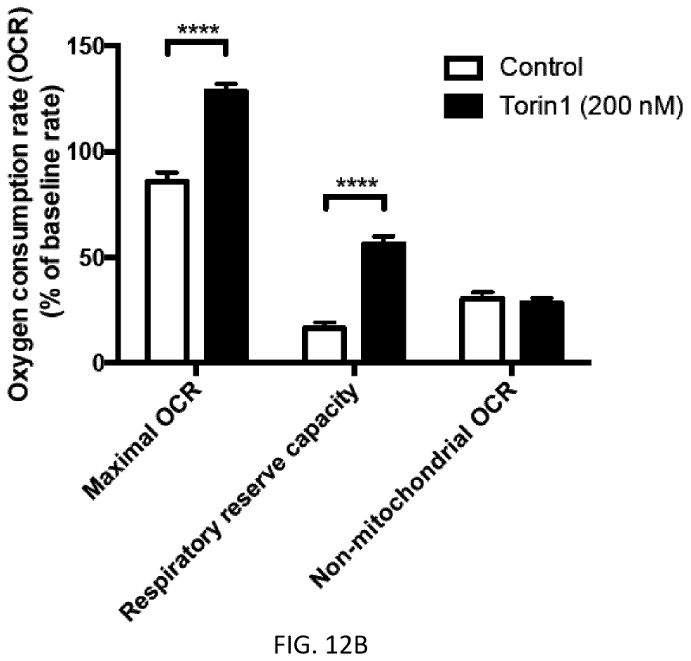

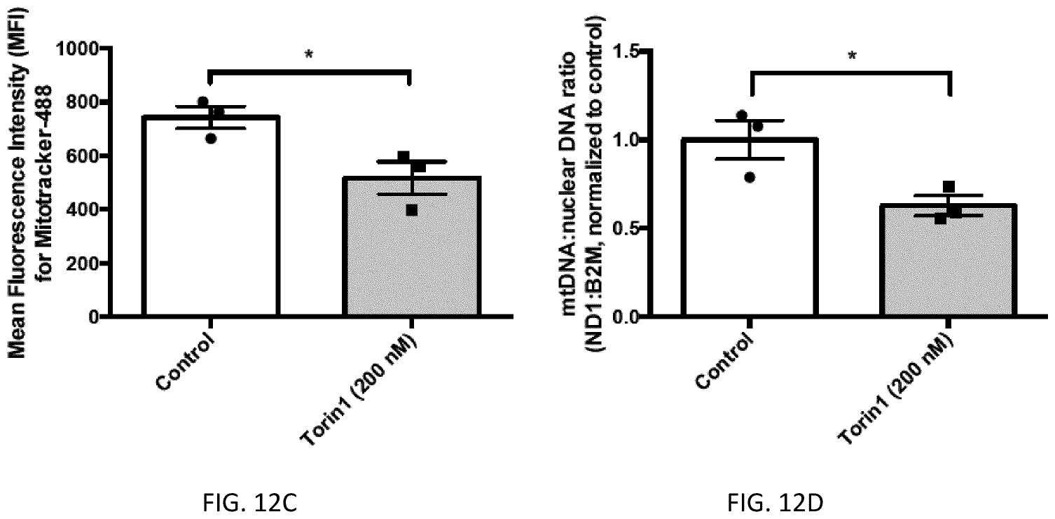

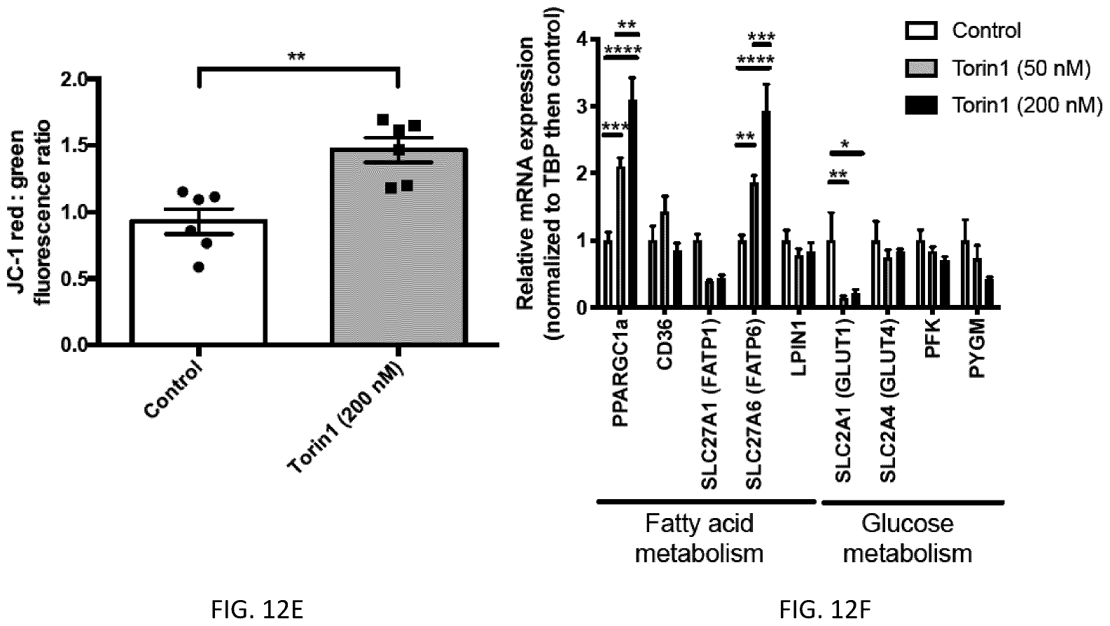

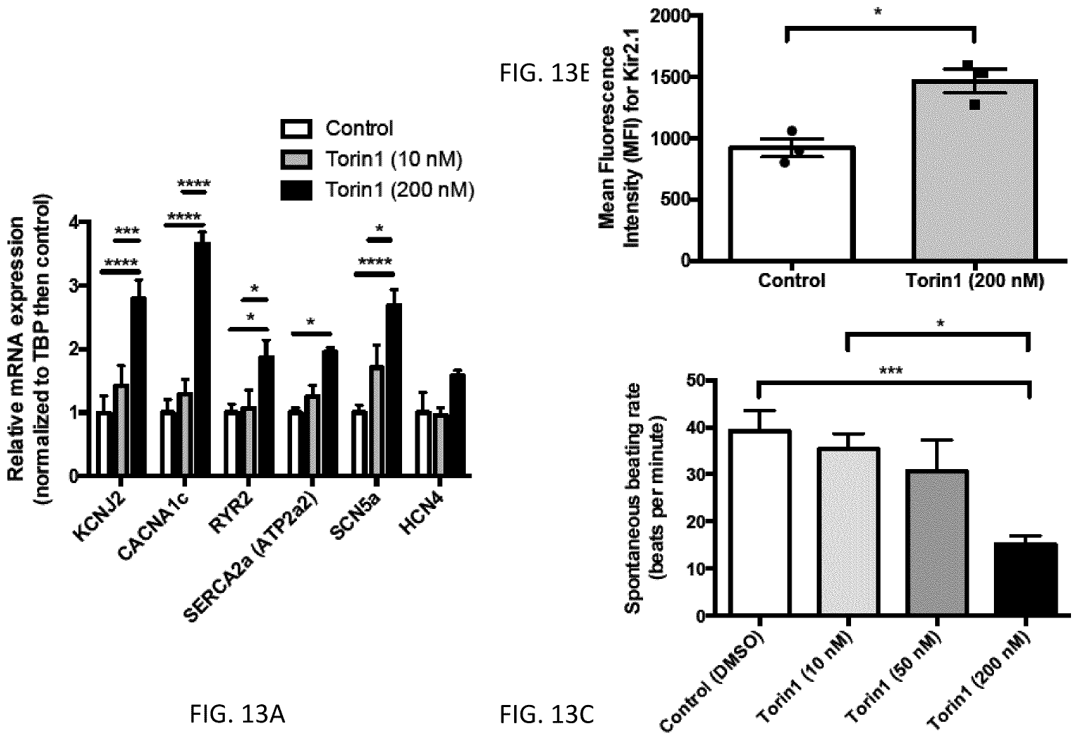

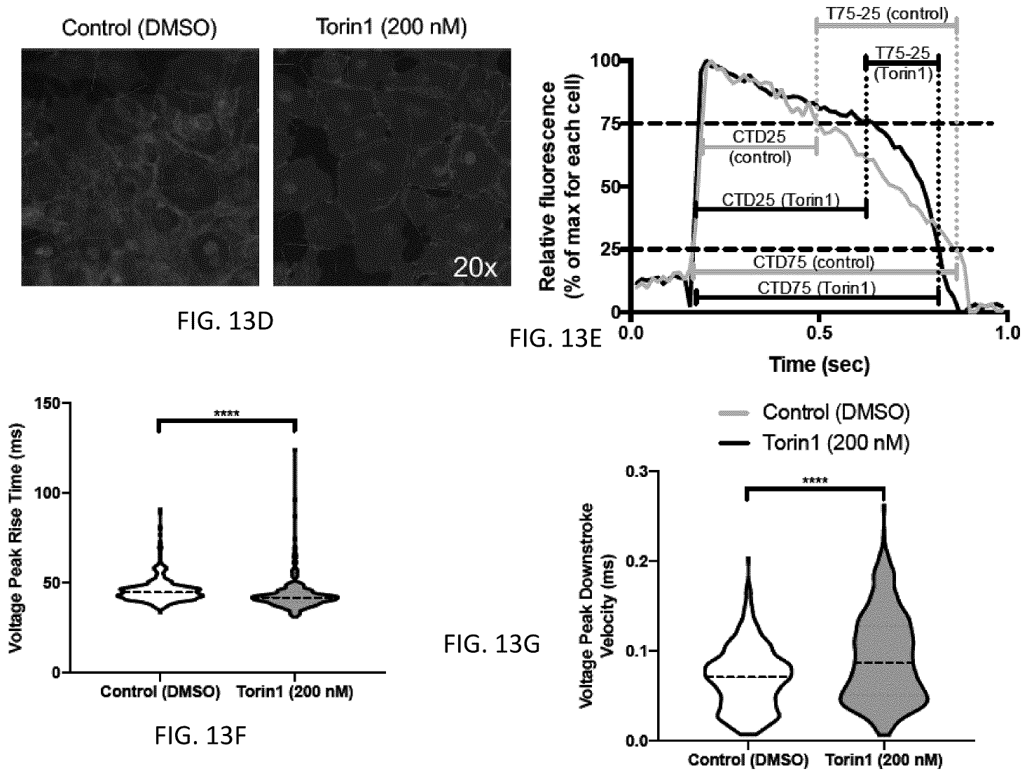

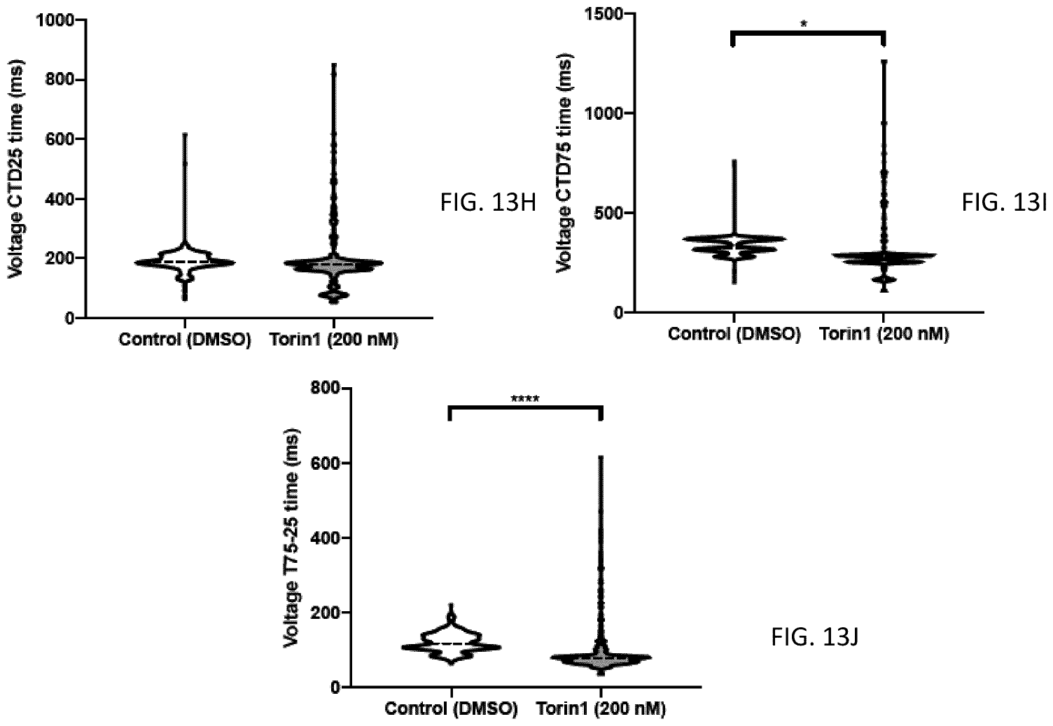

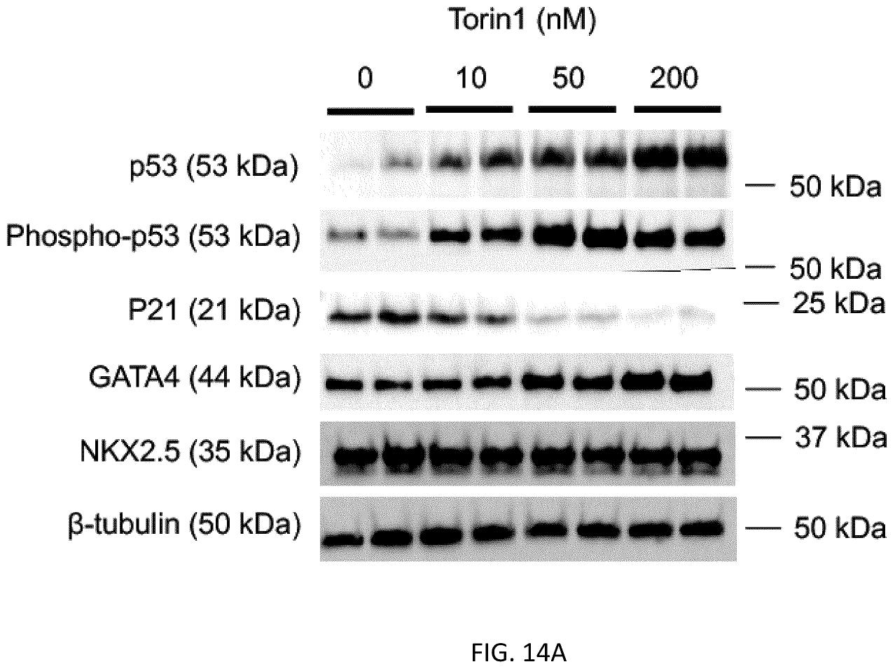

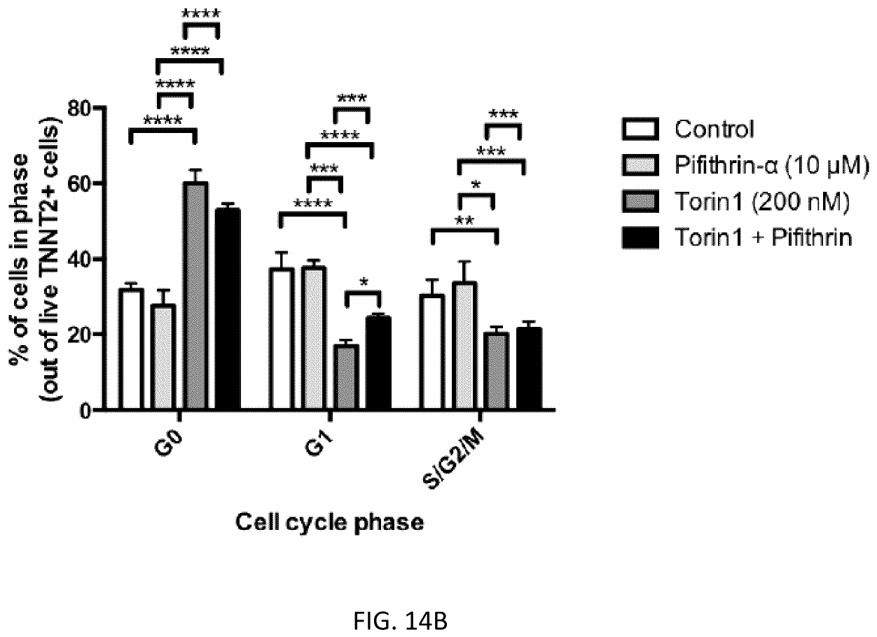

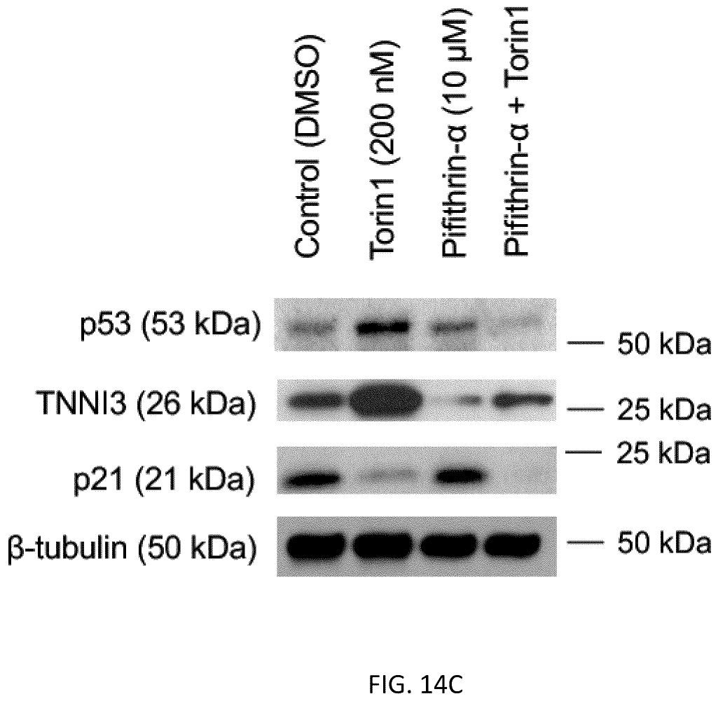

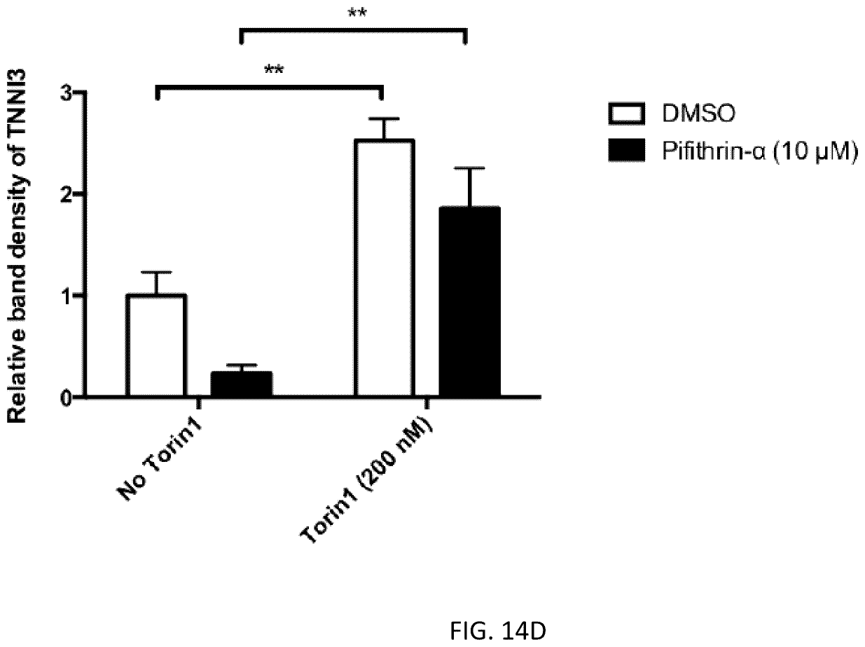

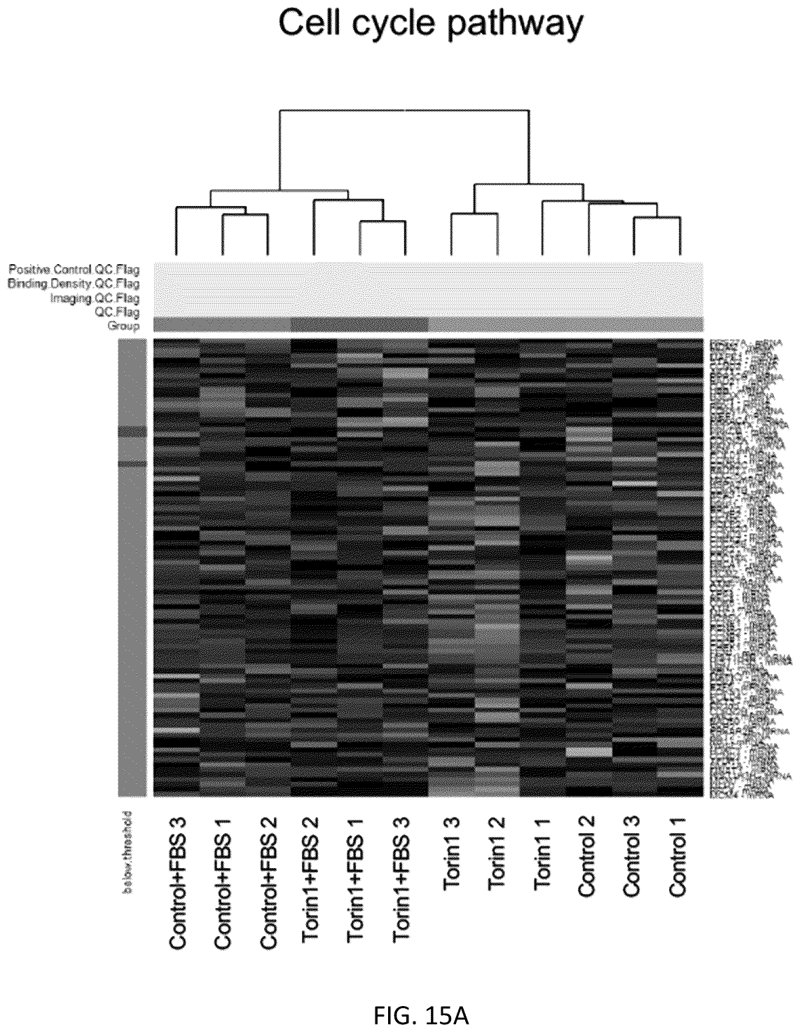

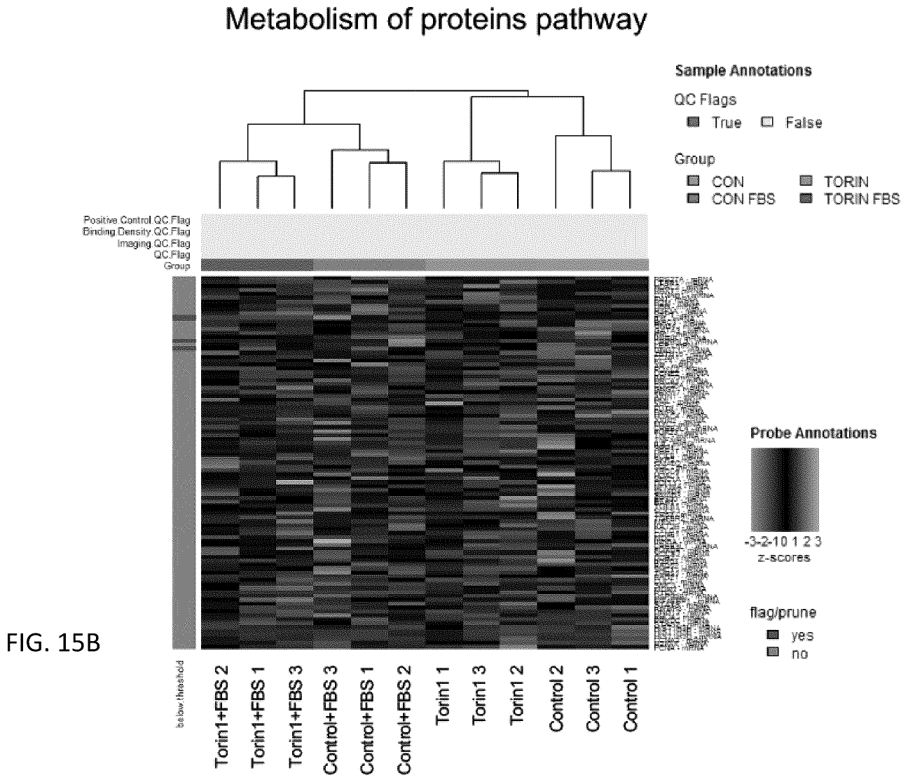

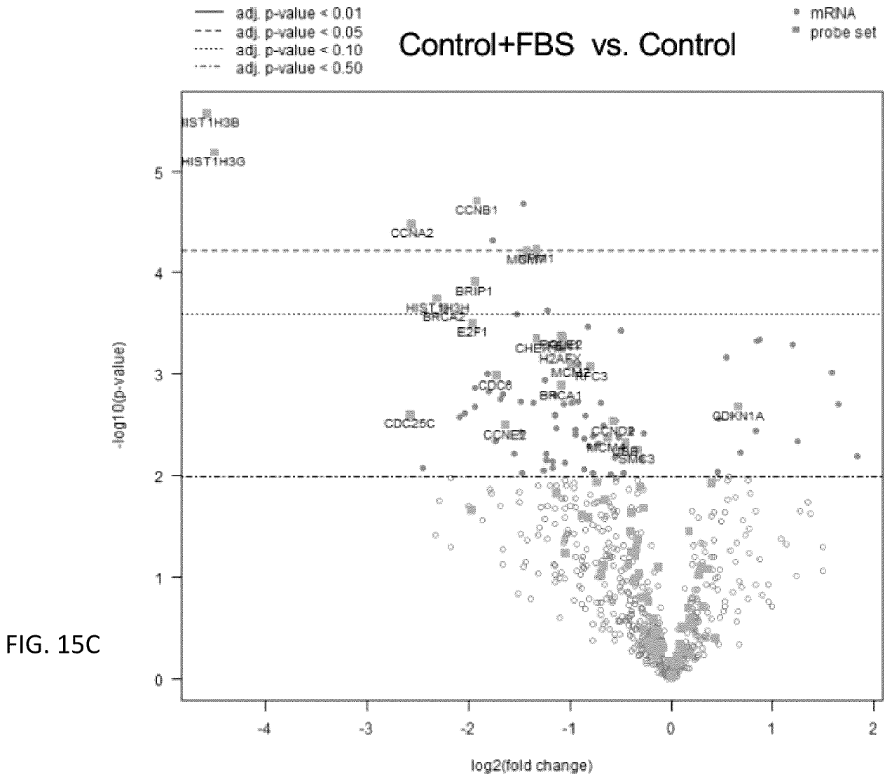

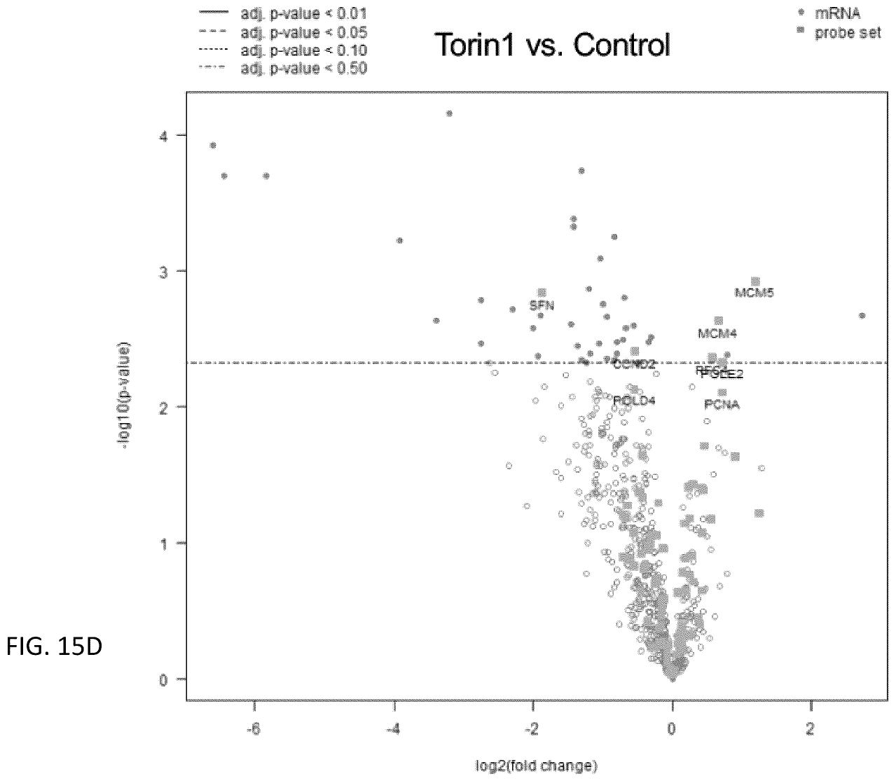

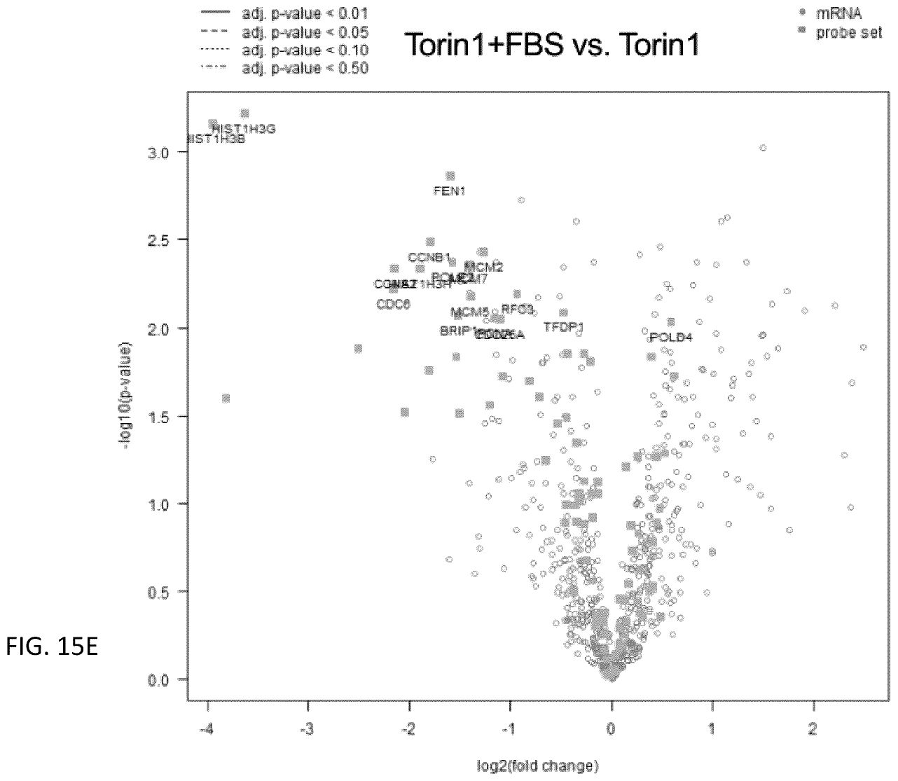

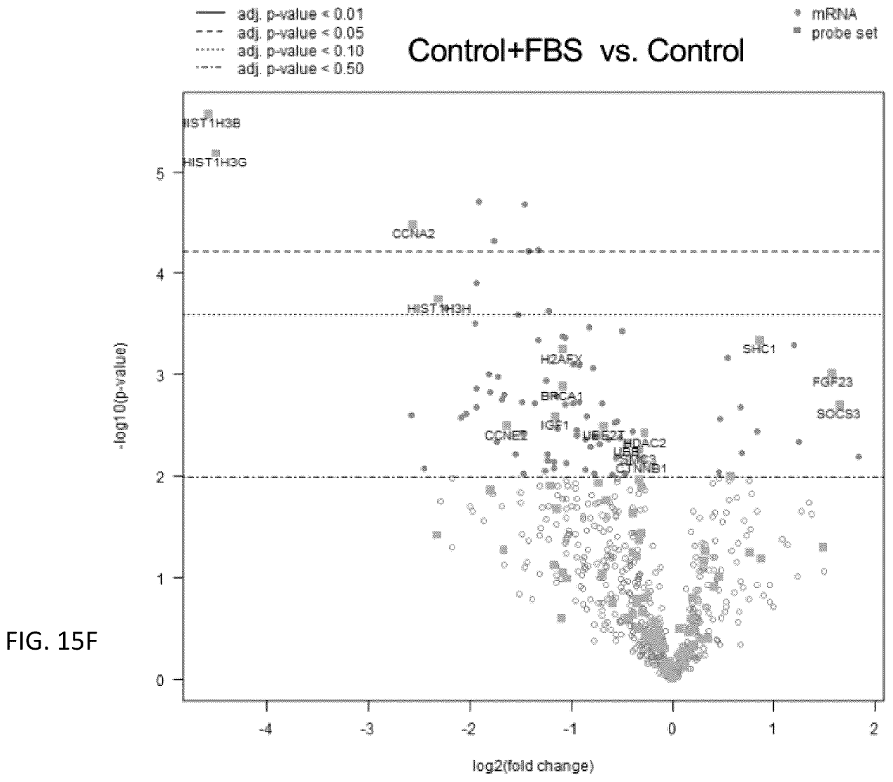

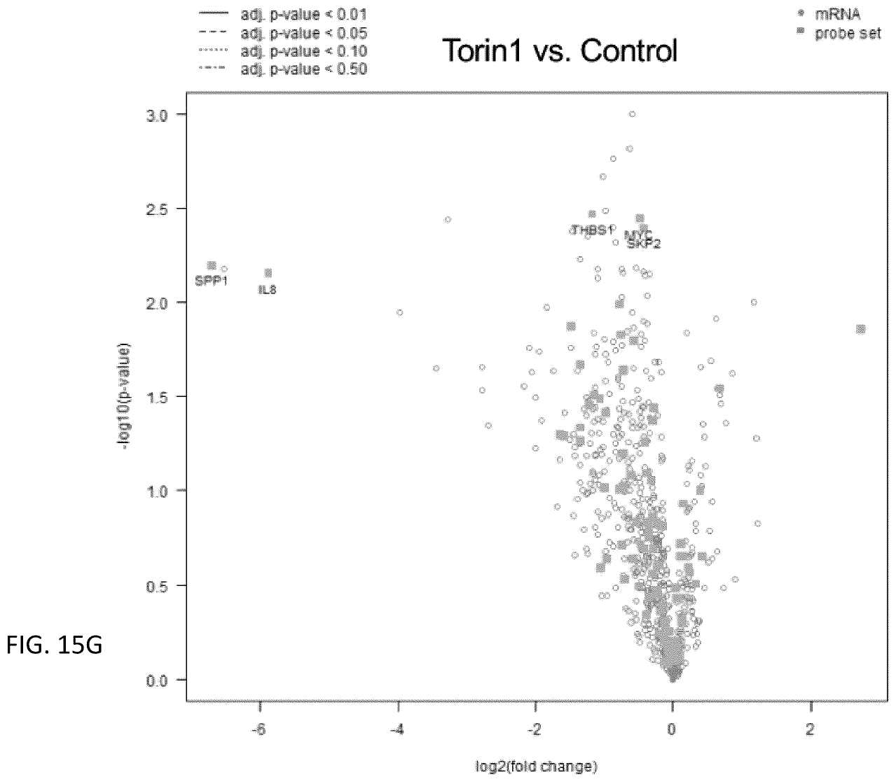

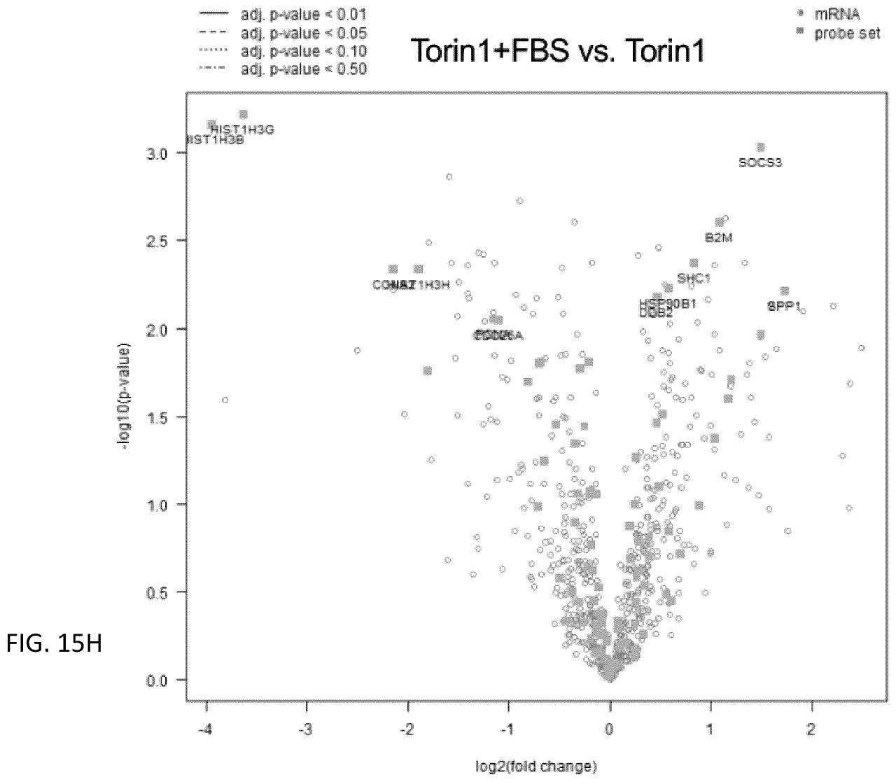

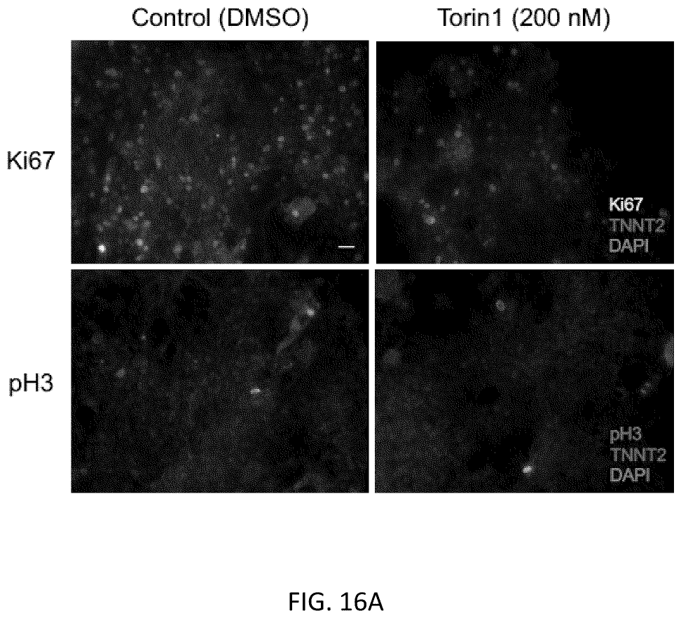

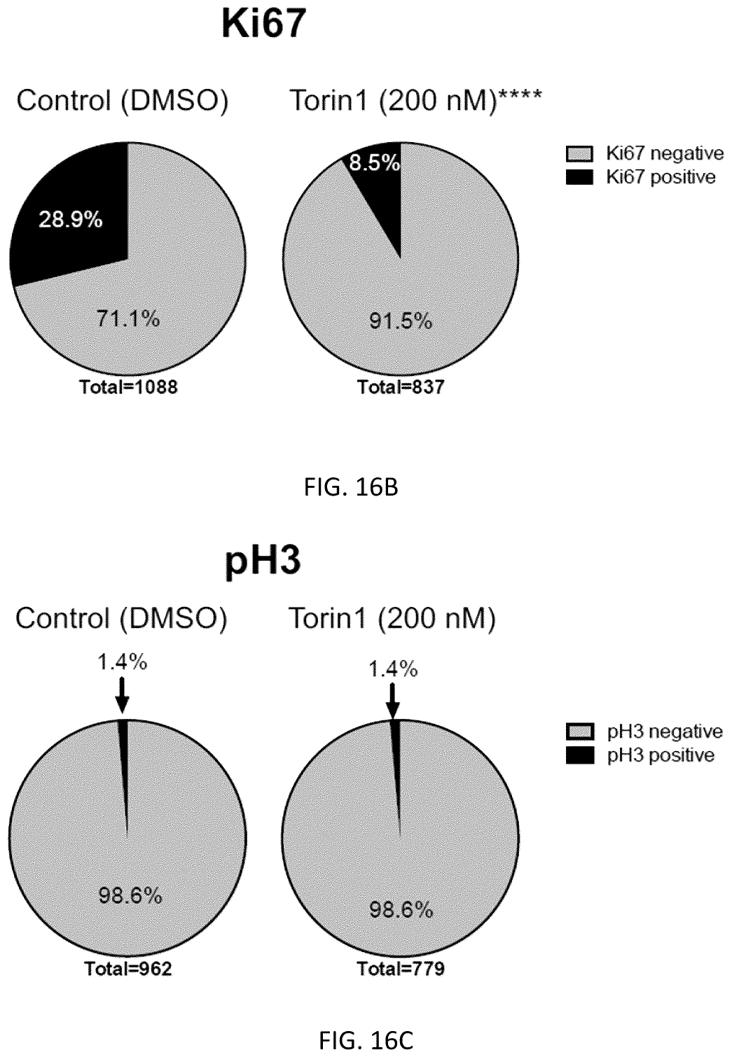

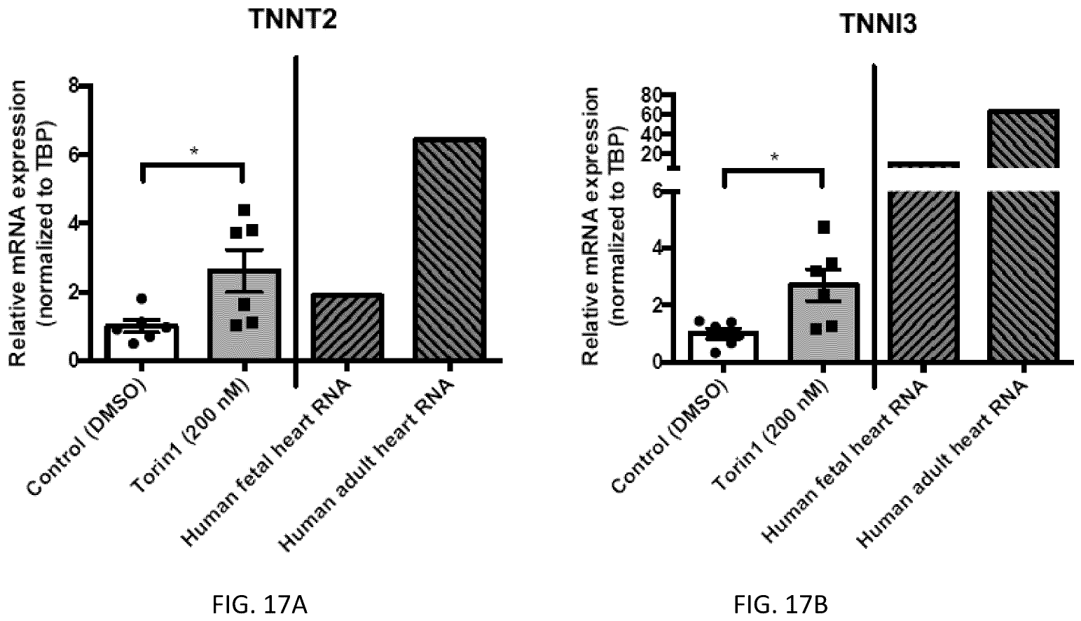

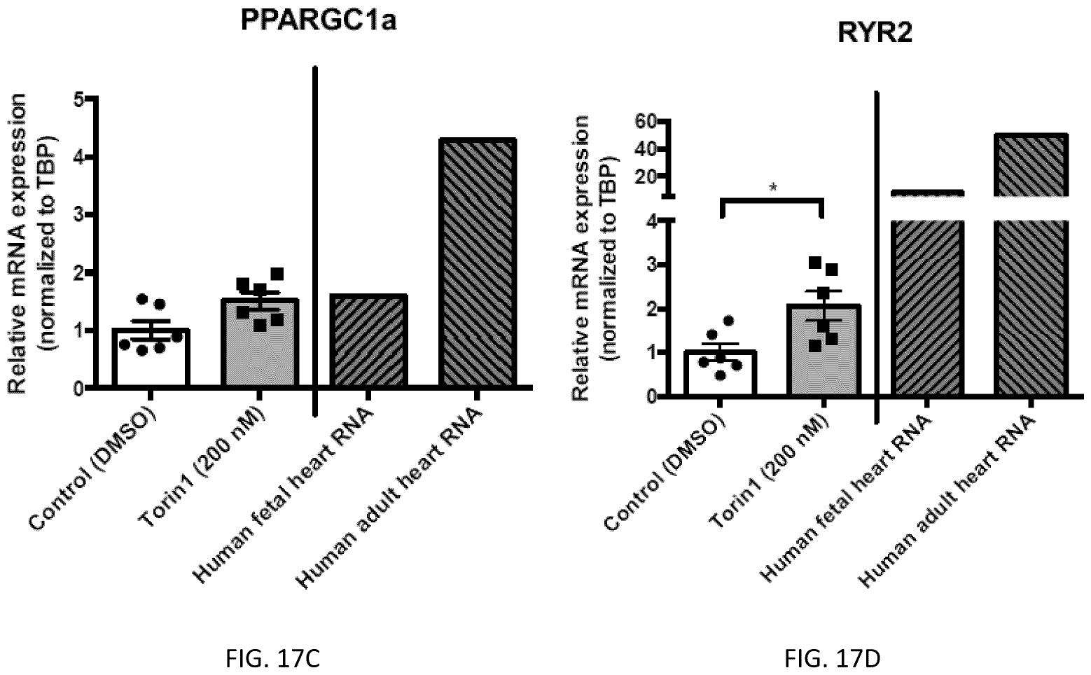

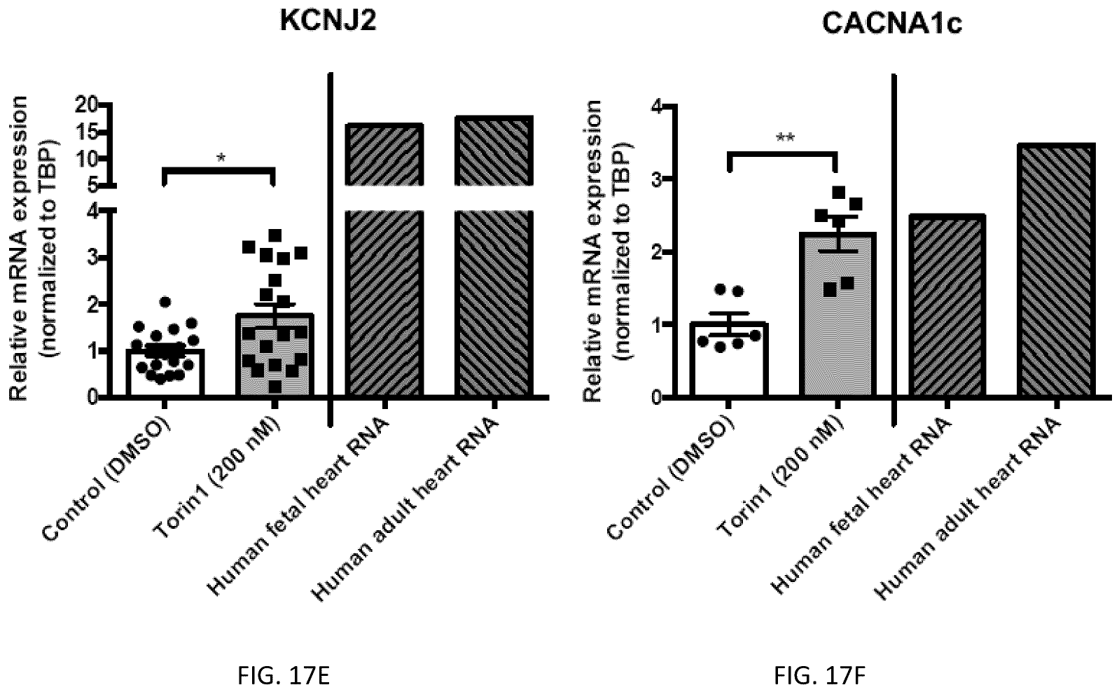

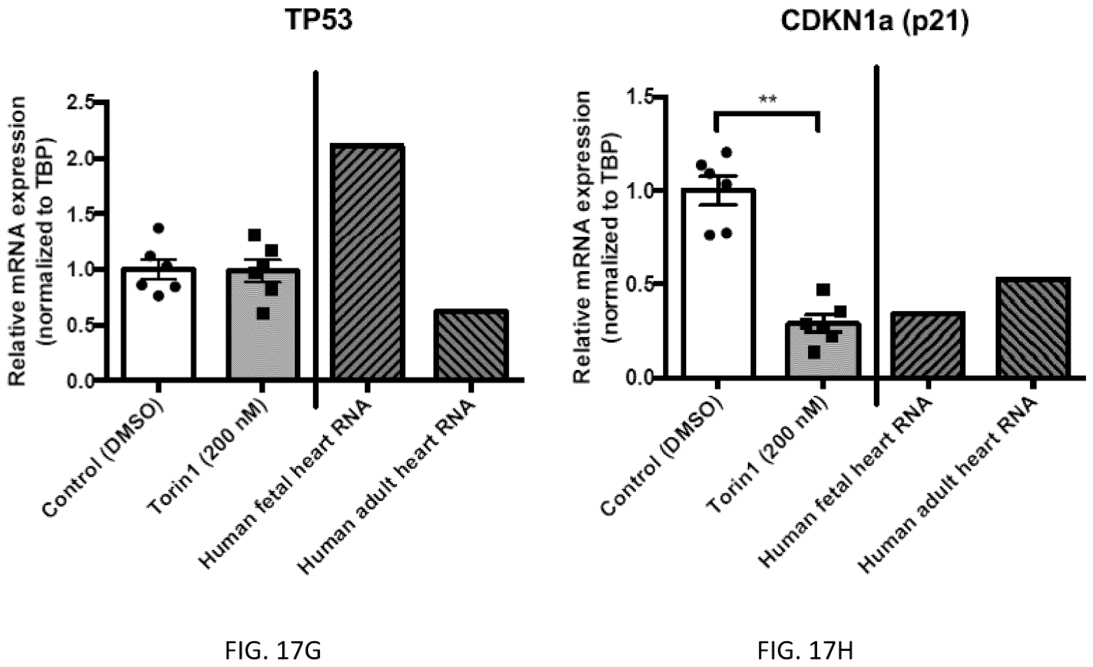

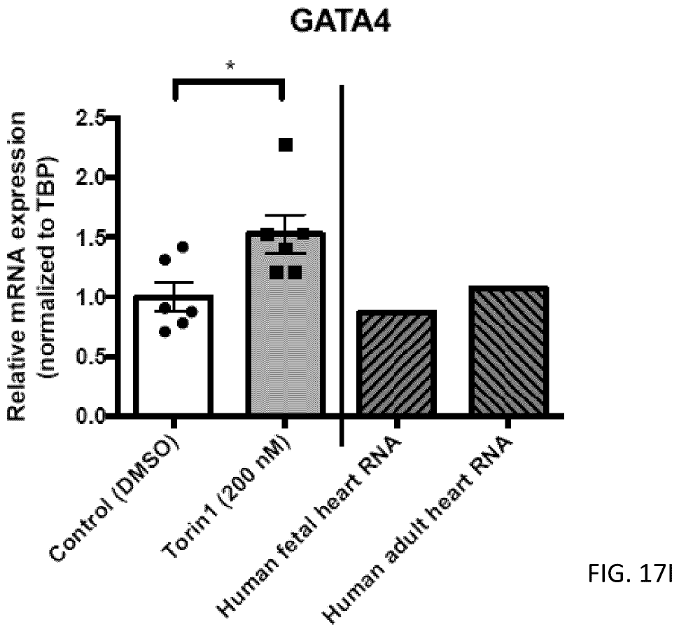

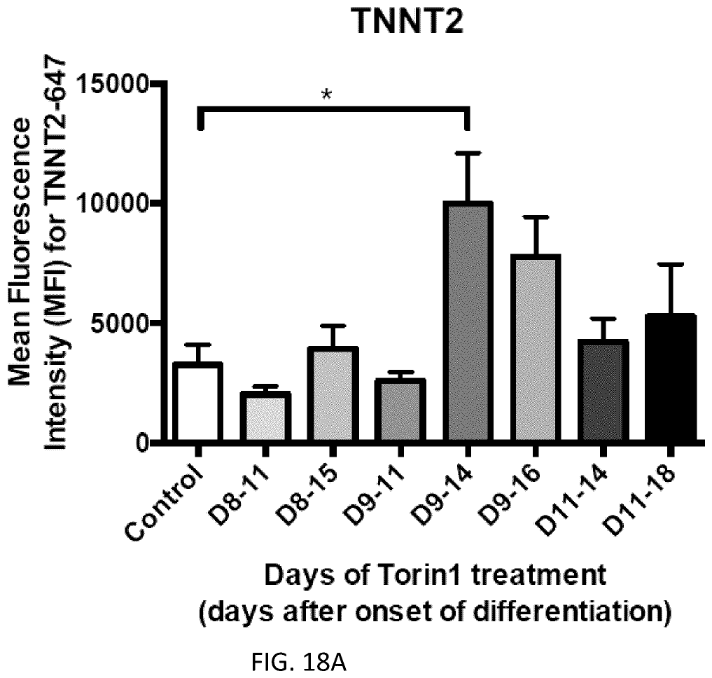

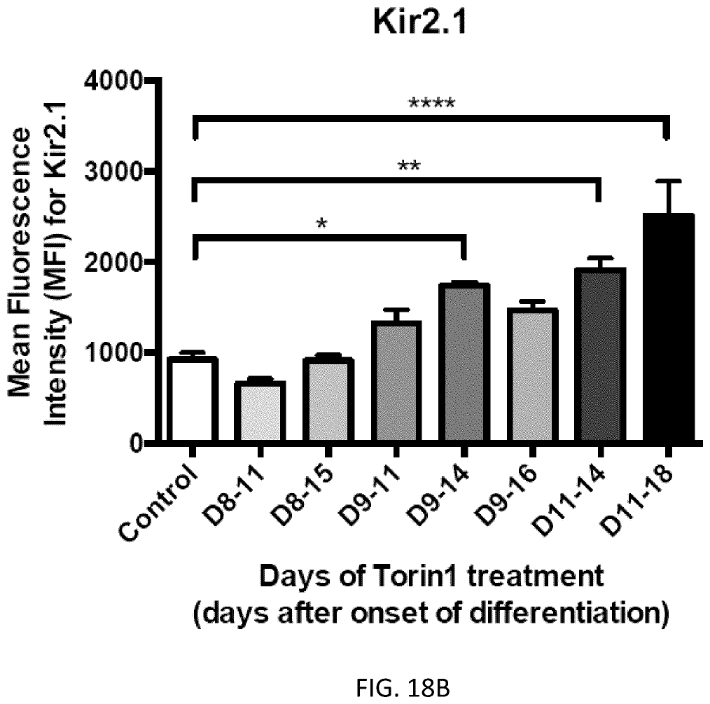

Treatment with 200 μM quercetin for 3 days significantly increased the mRNA expression of peroxisome proliferator-activated receptor gamma coactivator 1A (PPARGC1a) (FIG. 5), a regulator of mitochondrial biogenesis. PPARGC1a activation can stimulate cardiomyocyte maturation (10), suggesting that quercetin-treated cells may have a more mature metabolic phenotype.

### Summary

In summary, removal of senescent cells in 3D suspension culture using the senolytic, quercetin, increases expression of selected markers of PSC-CM maturation, including Kir2.1 and PPARGC1a. These results suggest that quercetin may improve maturation of surviving iPSC-CMs. Removal of senescent cells using senolytics may facilitate maturation of iPSC-CMs in culture prior to transplantation.

### REFERENCES

1. Chong J J, Yang X, Don C W, Minami E, Liu Y W, Weyers J J, Mahoney W M, Van Biber B, Cook S M, Palpant N J, Gantz J A, Fugate J A, Muskheli V, Gough G M, Vogel K W, Astley C A, Hotchkiss C E, Baldessari A, Pabon L, Reinecke H, Gill E A, Nelson V, Kiem H P, Laflamme M A, Murry C E. Human embryonic-stem-cell-derived cardiomyocytes regenerate non-human primate hearts. Nature. 2014; 510(7504):273-7. doi: 10.1038/nature13233. PubMed PMID: 24776797; PMCID: PMC4154594.
2. Yang X, Pabon L, Murry C E. Engineering adolescence: maturation of human pluripotent stem cell-derived cardiomyocytes. Circ Res. 2014; 114(3):511-23. doi: 10.1161/CIRCRESAHA.114.300558. PubMed PMID: 24481842; PMCID: PMC3955370.
3. Porrello E R, Mahmoud A I, Simpson E, Hill J A, Richardson J A, Olson E N, Sadek H A. Transient regenerative potential of the neonatal mouse heart. Science. 2011; 331(6020):1078-80. Epub 2011/02/26. doi: 10.1126/science.1200708. PubMed PMID: 21350179; PMCID: PMC3099478.
4. Yuan X, Braun T. Multimodal Regulation of Cardiac Myocyte Proliferation. Circ Res. 2017; 121(3):293-309. Epub 2017/07/22. doi: 10.1161/CIRCRESAHA.117.308428. PubMed PMID: 28729454.
5. Garbern J C, Helman A, Sereda R, Sarikhani M, Ahmed A, Escalante G O, Ogurlu R, Kim S L, Zimmerman J F, Cho A, MacQueen L, Bezzerides V J, Parker K K, Melton DA, Lee R T. Inhibition of mTOR Signaling Enhances Maturation of Cardiomyocytes Derived From Human-Induced Pluripotent Stem Cells via p53-Induced Quiescence. Circulation. 2020; 141(4):285-300. Epub 2019/11/12. doi: 10.1161/CIRCULATIONAHA.119.044205. PubMed PMID: 31707831; PMCID: PMC7009740.
6. Yao G. Modelling mammalian cellular quiescence. Interface Focus. 2014; 4(3):20130074. Epub 2014/06/07. doi: 10.1098/rsfs.2013.0074. PubMed PMID: 24904737; PMCID: PMC3996586.
7. Di Micco R, Krizhanovsky V, Baker D, d'Adda di Fagagna F. Cellular senescence in ageing: from mechanisms to therapeutic opportunities. Nat Rev Mol Cell Biol. 2021; 22(2):75-95. Epub 2020/12/18. doi: 10.1038/s41580-020-00314-w. PubMed PMID: 33328614.
8. Zhu Y, Tchkonia T, Pirtskhalava T, Gower A C, Ding H, Giorgadze N, Palmer A K, Ikeno Y, Hubbard G B, Lenburg M, O'Hara S P, LaRusso N F, Miller J D, Roos C M, Verzosa G C, LeBrasseur N K, Wren J D, Farr J N, Khosla S, Stout M B, McGowan S J, Fuhrmann-Stroissnigg H, Gurkar A U, Zhao J, Colangelo D, Dorronsoro A, Ling Y Y, Barghouthy A S, Navarro D C, Sano T, Robbins P D, Niedernhofer L J, Kirkland J L. The Achilles' heel of senescent cells: from transcriptome to senolytic drugs. Aging Cell. 2015; 14(4):644-58. doi: 10.1111/acel.12344.
9. Lian X, Zhang J, Azarin S M, Zhu K, Hazeltine L B, Bao X, Hsiao C, Kamp T J, Palecek S P. Directed cardiomyocyte differentiation from human pluripotent stem cells by modulating Wnt/beta-catenin signaling under fully defined conditions. Nat Protoc. 2013; 8(1):162-75. Epub 2012/12/22. doi: 10.1038/nprot.2012.150. PubMed PMID: 23257984; PMCID: PMC3612968.
10. Liu Y, Bai H, Guo F, Thai P N, Luo X, Zhang P, Yang C, Feng X, Zhu D, Guo J, Liang P, Xu Z, Yang H, Lu X. PGC-lalpha activator ZLN005 promotes maturation of cardiomyocytes derived from human embryonic stem cells. Aging (Albany NY). 2020; 12(8):7411-30. Epub 2020/04/29. doi: 10.18632/aging.103088. PubMed PMID: 32343674; PMCID: PMC7202542.

### Example 2: Electrical Maturation of Stem Cell-Derived Cardiomyocytes

### Executive Summary

Cardiovascular disease remains the leading cause of death worldwide. Allogeneic pluripotent stem cell PSC-derived cardiomyocytes (PSC-CMs) offer an off-the-shelf regenerative approach to treat heart failure. However, current protocols to differentiate human PSC-CMs generate immature cardiomyocytes that may increase the risk of life-threatening ventricular tachycardia (VT) likely due to a high resting membrane potential (RMP). In addition, adaptation of 2D protocols to three-dimensional (3D) suspension culture is necessary to facilitate scale-up, and this transition is not trivial. The goal of the current project is to adapt a 2D protocol into a 3D suspension culture protocol capable of generating electrically mature PSC-derived cardiomyocytes with a RMP≤−70 mV. Achieving this benchmark along with complete phenotypic characterization of 3D-generated cardiomyocytes is necessary to proceed with large animal testing and subsequent clinical translation and commercialization of this regenerative technology.

### Background

Following an acute myocardial infarction, the human heart loses approximately 1 billion or more cardiomyocytes (1). Allogeneic human pluripotent stem cell-derived cardiomyocytes (PSC-CMs) can offer an off-the-shelf regenerative approach to treat patients with systolic heart failure following a myocardial infarction. However, inadequate maturation of PSC-CMs may be a major barrier to clinical translation, as delivery of immature cardiomyocytes increases the risk of ventricular tachycardia (VT), a potentially life-threatening arrhythmia (2-4). It is thought that the increased risk of VT occurs because the PSC-CMs generated with most protocols are too immature, with PSC-CMs having a relatively high resting membrane potential (RMP) of around −50 to −60 mV (in contrast, adult ventricular cardiomyocytes have a RMP around −90 mV) (5). Inadequate electrical maturation of PSC-CMs is significantly hindering forward progress in developing PSC-CMs for clinical use, as strategies must first be developed to avoid the risk of VT.

Various strategies have been studied to enhance maturation, such as culture on substrates of different stiffness, long term culture (several months), electrical stimulation or incubation with small molecules (6-9). Achieving a RMP≤−70 mV in PSC-CMs in 3D suspension culture would overcome major barriers to clinical translation. Recently it was identified that induction of cellular quiescence via inhibition of the mechanistic target of rapamycin (mTOR) pathway with a single small molecule, Torin1, is capable of generating PSC-CMs in two-dimensional (2D) culture with a similar RMP in just 2 weeks (10), greatly simplifying the manufacturing process compared to other protocols that take 2 months to produce mature PSC-CMs (9).

### Significance

To develop replacement therapy using allogeneic human PSC-CMs as a viable therapeutic option, methods to culture large numbers of PSC-CMs with good manufacturing practices for off-the-shelf use must be developed. Maintenance of 2D cultures is labor-intensive with significant batch-to-batch variability (11). As such, many groups are transitioning to maintenance and differentiation of PSCs in three-dimensional (3D) bioreactor systems, which are more amenable to scale up, reduce labor time and small volume sampling allows for improved quality control (12). However, this shift from 2D to 3D culture alters the phenotype of the cells due to differential regulation of various signaling pathways in different culture geometries including mTOR (13). The field has yet to devise a uniform protocol that efficiently produces mature PSC-CMs in 3D. Overcoming this challenge is necessary before clinical translation and commercialization of such a therapy will be possible. Recently preliminary data suggest that mTOR inhibition has minimal effect on maturation in 3D culture. Additional preliminary data suggest that in 3D culture, other small molecules that can upregulate p53 and/or eliminate senescent cells may have a more pronounced effect on PSC-CM maturation.

The objectives of this project are:
1. To determine whether p53 activation with nutlin-3a enhances electrical maturation of iPSC-derived cardiomyocytes in 3D culture in vitro.
2. To determine whether elimination of senescent cells with the senolytic, quercetin, enhances electrical maturation of iPSC-derived cardiomyocytes in 3D culture in vitro.

### Preliminary Studies

### Nutlin-3a but not Torin1 Increases Expression of Kir2.1 and p53 in 3D Culture.

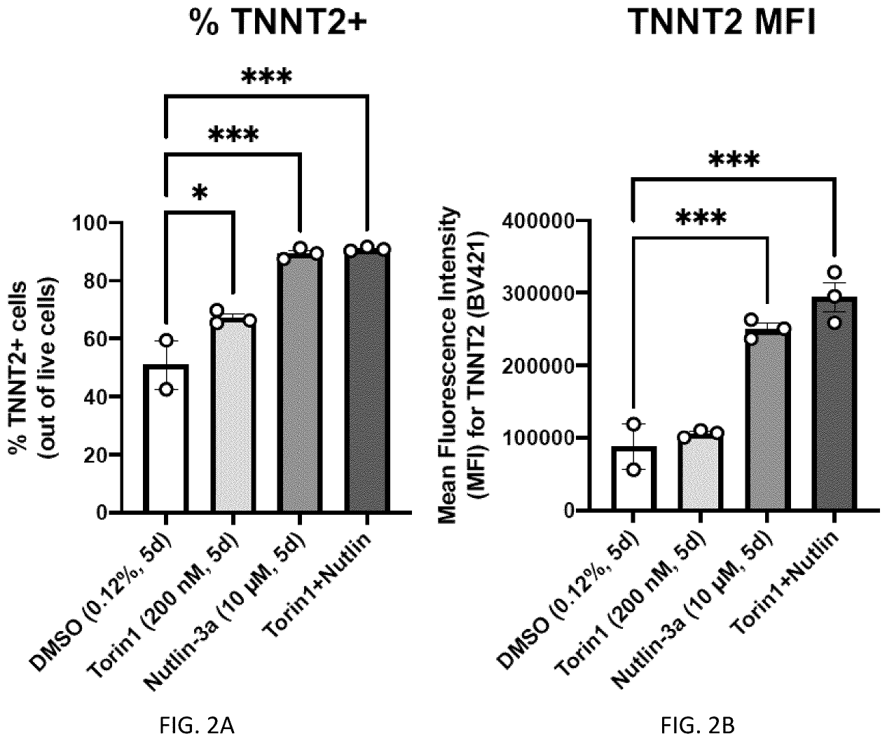

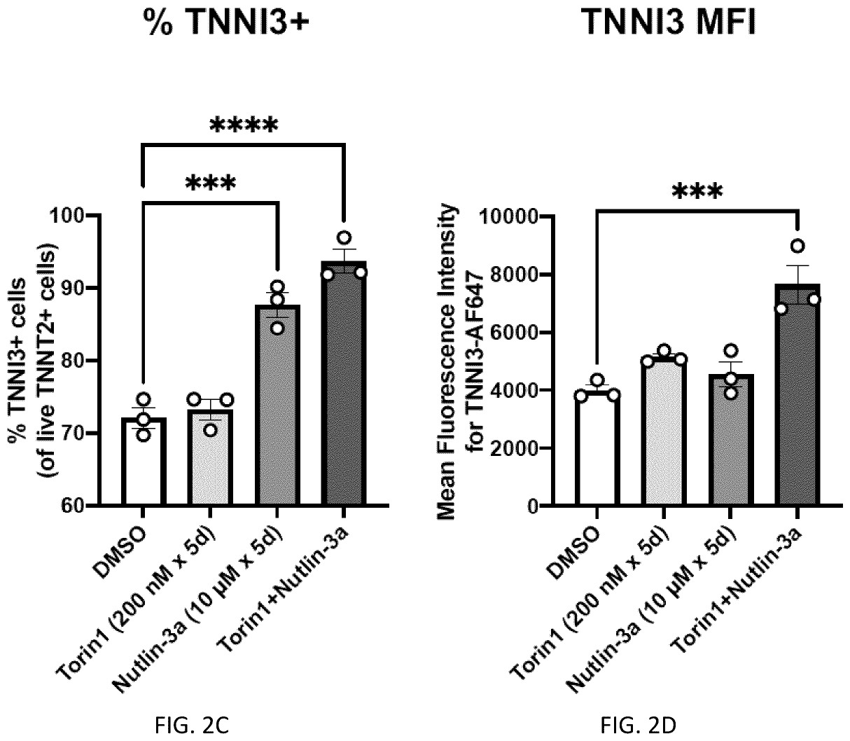

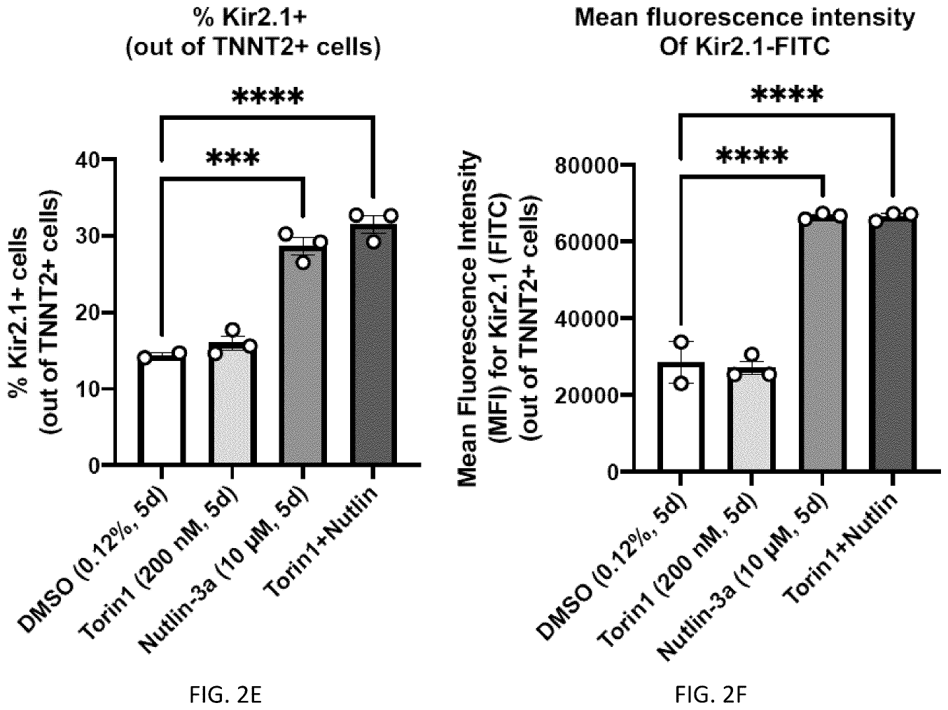

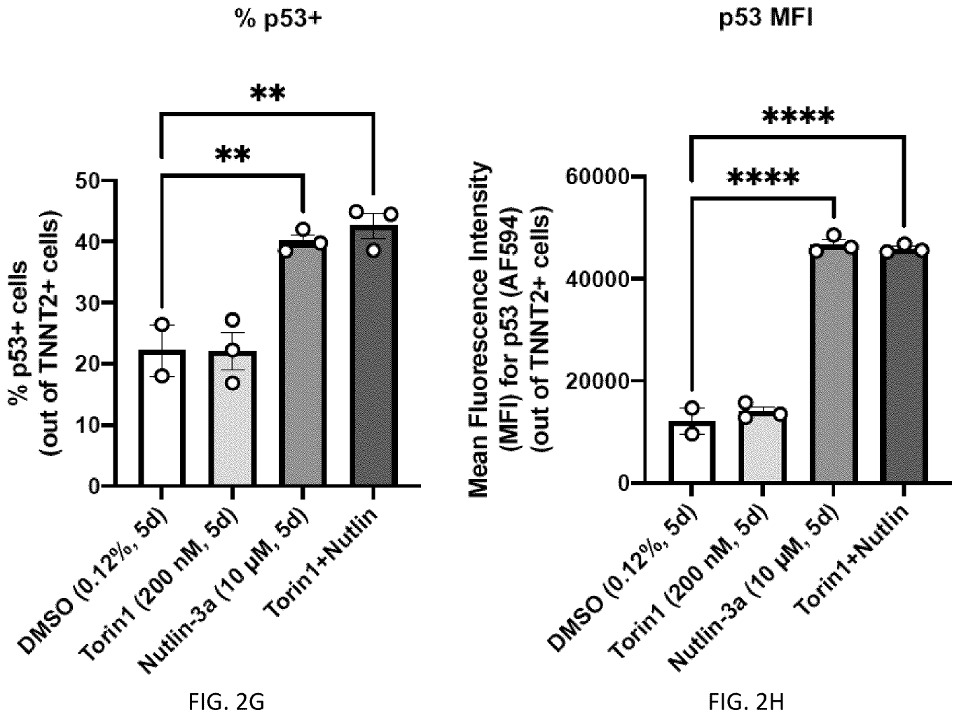

Kir2.1 is the ion channel largely responsible for maintaining the resting membrane potential via the inward rectifier current (IK1); at lower levels, abnormal membrane depolarization can increase the risk of ventricular arrhythmias (10, 11). Nutlin-3a but not Torin1 treatment of iPSC-CMs increases expression of Kir2.1 (FIGS. 2A-2H). Also, Nutlin-3a but not Torin1 increases expression of p53, which may support induction of a quiescent state (FIGS. 2A-2H). The preliminary data suggest that mTOR inhibition does not have a beneficial effect in 3D culture compared to 2D culture, possibly due to contact inhibition reducing mTOR activity (13).

### Quercetin Increases Expression of Kir2.1 and p53 in 3D Culture.

Quercetin treatment of iPSC-CMs increases expression of both Kir2.1 and p53 (FIGS. 3A-3D), providing evidence that quercetin may facilitate electrical maturation and quiescence of iPSC-CMs.

### Research Plan

It is proposed to develop a current Good Manufacturing Practice (cGMP)-complaint protocol capable of generating electrically-mature human PSC-CMs in 3D suspension culture through activation of p53 or elimination of senescent cells. A benchmark of RMP≤−70 mV will be used, which will demonstrate adequate improvement over current protocols to proceed with large animal studies to assess arrhythmia potential. Full phenotypic characterization will be performed including evaluating RNA and protein expression of sarcomere, ion channel and metabolic genes, quantifying contractility, electrophysiological properties, and oxygen consumption rate. A 3D suspension culture protocol has been optimized using Erlenmeyer flasks on a shaker platform. The protocol described herein can act as an adjunct to other differentiation protocols that can promote further maturation beyond the other protocols, with treatment starting after onset of PSC-CM beating.

### Rationale for Objective 1

Cellular quiescence is a resting state triggered by nutrient deprivation and is characterized by the ability to re-enter the cell cycle in response to appropriate stimuli (14). In contrast, cellular senescence is a state of irreversible cell cycle arrest associated with an aging or diseased phenotype, characterized by DNA damage, elevated reactive oxygen species levels, and expression of a senescence-associated secretory phenotype (SASP) that can lead to a pro-inflammatory phenotype in nearby cells (15). It was found that iPSC-CMs with a senescent phenotype are less mature, with lower expression of sarcomere or ion channel proteins seen in more mature cardiomyocytes. Upregulation of p53 may enhance expression maturation by supporting quiescence. It is proposed to generate electrically-mature human pluripotent stem cell-derived cardiomyocytes (PSC-CMs) through the use of the p53 activator, nutlin-3a, in 3D suspension culture.

### Rationale for Objective 2

Because SASP proteins such as interleukin-6 (IL-6) can have detrimental effects on nearby cells due to a paracrine effect, it will be tested whether removal of senescent cells with a senolytic can enhance the maturation of surviving cells. The senolytic, quercetin, is a flavonol found in foods such as apples that can induce apoptosis in senescent tumor cells (16). Whether quercetin can improve electrical maturation of iPSC-CMs by suppression of a senescent phenotype in 3D suspension culture will be evaluated.

### Experimental Design and Methods for Objectives 1 and 2

PSC-CMs will be treated in 3D culture with nutlin-3a (Objective 1) or quercetin (Objective 2). Contractile, metabolic, and electrical properties of the iPSC-CMs will be characterized to determine whether the proposed interventions improve maturation. The complete characterization of the electrical properties of the cells will be the focus. This will be important to understand whether the proposed interventions might reduce the risk of ventricular arrhythmias if these iPSC-CMs are implanted into a large animal in vivo model in future work.

Cell lines, differentiation and treatment. At least 3 human induced PSC (iPSC) lines (DiPS 1016 SevA, Gibco, UCSD142i-86-1) of at least 2 different origin cell types (e.g. fibroblast-derived or peripheral blood mononuclear cell-derived iPSCs) and from both male and female donors will be utilized for each experiment. This will provide confidence that our findings are robust across cell lines. Cell lines will be obtained from the Harvard Stem Cell Institute iPSC core facility (DiPS 1016 SevA), other academic cell distributors, established stem cell banks (such as WiCell for UCSD142i-86-1 line), or commercial vendors (Gibco). The differentiation protocol outlined by Lian et al (11) will be used and has been successfully adapted for both 2D and 3D culture. iPSC-CMs will be treated with either Nutlin-3a (Objective 1) or quercetin (Objective 2) at different time points and concentrations after onset of cardiomyocyte beating (˜day 7-9 of differentiation).

Cardiomyocyte characterization. To evaluate whether the proposed interventions affect iPSC-CM maturation, metabolic, contractile, and electrophysiological properties of iPSC-CMs will be quantified. mRNA expression will be evaluated via single cell RNAseq and protein expression of selected metabolic (GLUT1-4, FATP1-6, PPARa, PPARg, PGC1a), contractile (TNNT2, TNNI3, MYH6, and MYH7), and ion channel (KCNJ2, CACNA1c, SERCA2a, RYR2, and HCN4) proteins via western analysis. Mitochondrial membrane polarization will be quantified with MitoTracker Red CMXRos (Thermo). The Seahorse XFe96 Analyzer will be used to assess oxygen consumption rate (OCR) and extracellular acidification rate (ECAR). Contractility in 2D culture will be quantified using a video-based analysis and accompanying MATLAB script (17). Calcium transients will be analyzed using Fluo-4 (18) and action potential duration will be analyzed using FluoVolt technology (19) using a Vala kinetic image cytometer (KIC) (20). Patch clamp analysis will be performed to quantify resting membrane potential and I_{K1}.

### REFERENCE LIST

1. Laflamme M A, Murry C E. Regenerating the heart. Nat Biotechnol. 2005; 23(7):845-56. Epub 2005/07/09. doi: 10.1038/nbt1117. PubMed PMID: 16003373.
2. Chong J J, Yang X, Don C W, Minami E, Liu Y W, Weyers J J, Mahoney W M, Van Biber B, Cook S M, Palpant N J, Gantz J A, Fugate J A, Muskheli V, Gough G M, Vogel K W, Astley C A, Hotchkiss C E, Baldessari A, Pabon L, Reinecke H, Gill E A, Nelson V, Kiem H P, Laflamme M A, Murry C E. Human embryonic-stem-cell-derived cardiomyocytes regenerate non-human primate hearts. Nature. 2014; 510(7504):273-7. doi: 10.1038/nature13233. PubMed PMID: 24776797; PMCID: PMC4154594.
3. Liu Y W, Chen B, Yang X, Fugate J A, Kalucki F A, Futakuchi-Tsuchida A, Couture L, Vogel K W, Astley C A, Baldessari A, Ogle J, Don C W, Steinberg Z L, Seslar S P, Tuck S A, Tsuchida H, Naumova A V, Dupras S K, Lyu M S, Lee J, Hailey D W, Reinecke H, Pabon L, Fryer B H, MacLellan W R, Thies R S, Murry C E. Human embryonic stem cell-derived cardiomyocytes restore function in infarcted hearts of non-human primates. Nat Biotechnol. 2018; 36(7):597-605. Epub 2018/07/04. doi: 10.1038/nbt.4162. PubMed PMID: 29969440; PMCID: PMC6329375.
4. Romagnuolo R, Masoudpour H, Porta-Sanchez A, Qiang B, Barry J, Laskary A, Qi X, Masse S, Magtibay K, Kawajiri H, Wu J, Valdman Sadikov T, Rothberg J, Panchalingam K M, Titus E, Li R K, Zandstra P W, Wright G A, Nanthakumar K, Ghugre N R, Keller G, Laflamme M A. Human Embryonic Stem Cell-Derived Cardiomyocytes Regenerate the Infarcted Pig Heart but Induce Ventricular Tachyarrhythmias. Stem Cell Reports. 2019; 12:967-81. Epub 2019/05/06. doi: 10.1016/j.stemcr.2019.04.005. PubMed PMID: 31056479.
5. Karbassi E, Fenix A, Marchiano S, Muraoka N, Nakamura K, Yang X, Murry C E. Cardiomyocyte maturation: advances in knowledge and implications for regenerative medicine. Nat Rev Cardiol. 2020; 17(6):341-59. Epub 2020/02/06. doi: 10.1038/s41569-019-0331-x. PubMed PMID: 32015528; PMCID: PMC7239749.
6. Montessuit C, Palma T, Viglino C, Pellieux C, Lerch R. Effects of insulin-like growth factor-I on the maturation of metabolism in neonatal rat cardiomyocytes. Pflugers Arch. 2006; 452(4):380-6. doi: 10.1007/s00424-006-0059-4. PubMed PMID: 16586094.
7. Yang X, Rodriguez M, Pabon L, Fischer K A, Reinecke H, Regnier M, Sniadecki N J, Ruohola-Baker H, Murry C E. Tri-iodo-1-thyronine promotes the maturation of human cardiomyocytes-derived from induced pluripotent stem cells. J Mol Cell Cardiol. 2014; 72:296-304. doi: 10.1016/j.yjmcc.2014.04.005. PubMed PMID: 24735830; PMCID: PMC4041732.
8. Yang X, Pabon L, Murry C E. Engineering adolescence: maturation of human pluripotent stem cell-derived cardiomyocytes. Circ Res. 2014; 114(3):511-23. doi: 10.1161/CIRCRESAHA.114.300558. PubMed PMID: 24481842; PMCID: PMC3955370.
9. Feyen D A M, McKeithan W L, Bruyneel A A N, Spiering S, Hormann L, Ulmer B, Zhang H, Briganti F, Schweizer M, Hegyi B, Liao Z, Polonen R P, Ginsburg K S, Lam C K, Serrano R, Wahlquist C, Kreymerman A, Vu M, Amatya P L, Behrens C S, Ranjbarvaziri S, Maas R G C, Greenhaw M, Bernstein D, Wu J C, Bers D M, Eschenhagen T, Metallo C M, Mercola M. Metabolic Maturation Media Improve Physiological Function of Human iPSC-Derived Cardiomyocytes. Cell Rep. 2020; 32(3):107925. Epub 2020/07/23. doi: 10.1016/j.celrep.2020.107925. PubMed PMID: 32697997.
10. Garbern J C, Helman A, Sereda R, Sarikhani M, Ahmed A, Escalante G O, Ogurlu R, Kim S L, Zimmerman J F, Cho A, MacQueen L, Bezzerides V J, Parker K K, Melton D A, Lee R T. Inhibition of mTOR Signaling Enhances Maturation of Cardiomyocytes Derived From Human-Induced Pluripotent Stem Cells via p53-Induced Quiescence. Circulation. 2020; 141(4):285-300. Epub 2019/11/12. doi: 10.1161/CIRCULATIONAHA.119.044205. PubMed PMID: 31707831; PMCID: PMC7009740.
11. Lian X, Zhang J, Azarin S M, Zhu K, Hazeltine L B, Bao X, Hsiao C, Kamp T J, Palecek S P. Directed cardiomyocyte differentiation from human pluripotent stem cells by modulating Wnt/beta-catenin signaling under fully defined conditions. Nat Protoc. 2013; 8(1):162-75. Epub 2012/12/22. doi: 10.1038/nprot.2012.150. PubMed PMID: 23257984; PMCID: PMC3612968.
12. Kempf H, Kropp C, Olmer R, Martin U, Zweigerdt R. Cardiac differentiation of human pluripotent stem cells in scalable suspension culture. Nat Protoc. 2015; 10(9):1345-61. doi: 10.1038/nprot.2015.089. PubMed PMID: 26270394.
13. Riedl A, Schlederer M, Pudelko K, Stadler M, Walter S, Unterleuthner D, Unger C, Kramer N, Hengstschlager M, Kenner L, Pfeiffer D, Krupitza G, Dolznig H. Comparison of cancer cells in 2D vs 3D culture reveals differences in AKT-mTOR-S6K signaling and drug responses. J Cell Sci. 2017; 130(1):203-18. Epub 2016/09/25. doi: 10.1242/jcs.188102. PubMed PMID: 27663511.
14. Yao G. Modelling mammalian cellular quiescence. Interface Focus. 2014; 4(3):20130074. Epub 2014/06/07. doi: 10.1098/rsfs.2013.0074. PubMed PMID: 24904737; PMCID: PMC3996586.
15. Di Micco R, Krizhanovsky V, Baker D, d'Adda di Fagagna F. Cellular senescence in ageing: from mechanisms to therapeutic opportunities. Nat Rev Mol Cell Biol. 2021; 22(2):75-95. Epub 2020/12/18. doi: 10.1038/s41580-020-00314-w. PubMed PMID: 33328614.
16. Zhu Y, Tchkonia T, Pirtskhalava T, Gower A C, Ding H, Giorgadze N, Palmer A K, Ikeno Y, Hubbard G B, Lenburg M, O'Hara S P, LaRusso N F, Miller J D, Roos C M, Verzosa G C, LeBrasseur N K, Wren J D, Farr J N, Khosla S, Stout M B, McGowan S J, Fuhrmann-Stroissnigg H, Gurkar A U, Zhao J, Colangelo D, Dorronsoro A, Ling Y Y, Barghouthy A S, Navarro D C, Sano T, Robbins P D, Niedernhofer L J, Kirkland J L. The Achilles' heel of senescent cells: from transcriptome to senolytic drugs. Aging Cell. 2015; 14(4):644-58. doi: 10.1111/acel.12344.
17. Huebsch N, Loskill P, Mandegar M A, Marks N C, Sheehan A S, Ma Z, Mathur A, Nguyen T N, Yoo J C, Judge L M, Spencer C I, Chukka A C, Russell C R, So P L, Conklin B R, Healy K E. Automated Video-Based Analysis of Contractility and Calcium Flux in Human-Induced Pluripotent Stem Cell-Derived Cardiomyocytes Cultured over Different Spatial Scales. Tissue Eng Part C Methods. 2015; 21(5):467-79. Epub 2014/10/22. doi: 10.1089/ten.TEC.2014.0283. PubMed PMID: 25333967; PMCID: PMC4410286.
18. Ahola A, Polonen R P, Aalto-Setala K, Hyttinen J. Simultaneous Measurement of Contraction and Calcium Transients in Stem Cell Derived Cardiomyocytes. Ann Biomed Eng. 2018; 46(1):148-58. Epub 2017/10/05. doi: 10.1007/s10439-017-1933-2. PubMed PMID: 28975460; PMCID: PMC5754453.
19. Bedut S, Seminatore-Nole C, Lamamy V, Caignard S, Boutin J A, Nosjean O, Stephan J P, Coge F. Highthroughput drug profiling with voltage- and calcium-sensitive fluorescent probes in human iPSC-derived cardiomyocytes. Am J Physiol Heart Circ Physiol. 2016; 311(1):H44-53. Epub 2016/05/21. doi: 10.1152/ajpheart.00793.2015. PubMed PMID: 27199128.
20. Lu H R, Whittaker R, Price J H, Vega R, Pfeiffer E R, Cerignoli F, Towart R, Gallacher D J. High Throughput Measurement of Ca++ Dynamics in Human Stem Cell-Derived Cardiomyocytes by Kinetic Image Cytometery: A Cardiac Risk Assessment Characterization Using a Large Panel of Cardioactive and Inactive Compounds. Toxicol Sci. 2015; 148(2):503-16. Epub 2015/09/12. doi: 10.1093/toxsci/kfv201. PubMed PMID: 26358003.
21. Tisato V, Voltan R, Gonelli A, Secchiero P, Zauli G. MDM2/X inhibitors under clinical evaluation: perspectives for the management of hematological malignancies and pediatric cancer. J Hematol Oncol. 2017; 10(1):133. Epub 2017/07/05. doi: 10.1186/s13045-017-0500-5. PubMed PMID: 28673313; PMCID: PMC5496368.
22. Han M K, Barreto T A, Martinez F J, Comstock A T, Sajjan U S. Randomised clinical trial to determine the safety of quercetin supplementation in patients with chronic obstructive pulmonary disease. BMJ Open Respir Res. 2020; 7(1). Epub 2020/02/20. doi: 10.1136/bmjresp-2018-000392. PubMed PMID: 32071149; PMCID: PMC7047491.
23. Dehghani F, Sezavar Seyedi Jandaghi S H, Janani L, Sarebanhassanabadi M, Emamat H, Vafa M. Effects of quercetin supplementation on inflammatory factors and quality of life in post-myocardial infarction patients: A double blind, placebo-controlled, randomized clinical trial. Phytother Res. 2020. Epub 2020/11/21. doi: 10.1002/ptr.6955. PubMed PMID: 33216421.
24. Aucoin M, Cooley K, Saunders P R, Cardozo V, Remy D, Cramer H, Neyre Abad C, Hannan N. The effect of quercetin on the prevention or treatment of COVID-19 and other respiratory tract infections in humans: A rapid review. Adv Integr Med. 2020; 7(4):247-51. Epub 2020/08/25. doi: 10.1016/j.aimed.2020.07.007. PubMed PMID: 32837891; PMCID: PMC7392107.
25. Administration USFaD. Cellular & Gene Therapy Products 2021. Available from: https://www.fda.gov/vaccines-blood-biologics/cellular-gene-therapy-products.
26. Shiba Y, Fernandes S, Zhu W Z, Filice D, Muskheli V, Kim J, Palpant N J, Gantz J, Moyes K W, Reinecke H, Van Biber B, Dardas T, Mignone J L, Izawa A, Hanna R, Viswanathan M, Gold J D, Kotlikoff M I, Sarvazyan N, Kay M W, Murry C E, Laflamme M A. Human ES-cell-derived cardiomyocytes electrically couple and suppress arrhythmias in injured hearts. Nature. 2012; 489(7415):322-5. Epub 2012/08/07. doi: 10.1038/nature11317. PubMed PMID: 22864415; PMCID: PMC3443324.
27. Marin V, Cribioli E, Philip B, Tettamanti S, Pizzitola I, Biondi A, Biagi E, Pule M. Comparison of different suicide-gene strategies for the safety improvement of genetically manipulated T cells. Hum Gene Ther Methods. 2012; 23(6):376-86. Epub 2012/11/29. doi: 10.1089/hgtb.2012.050. PubMed PMID: 23186165; PMCID: PMC4015080.
28. Sulkowski M, Konieczny P, Chlebanowska P, Majka M. Introduction of Exogenous HSV-TK Suicide Gene Increases Safety of Keratinocyte-Derived Induced Pluripotent Stem Cells by Providing Genetic “Emergency Exit” Switch. Int J Mol Sci. 2018; 19(1). Epub 2018/01/10. doi: 10.3390/ijms19010197. PubMed PMID: 29315221; PMCID: PMC5796146.
29. Di Stasi A, Tey S K, Dotti G, Fujita Y, Kennedy-Nasser A, Martinez C, Straathof K, Liu E, Durett A G, Grilley B, Liu H, Cruz C R, Savoldo B, Gee A P, Schindler J, Krance R A, Heslop H E, Spencer D M, Rooney C M, Brenner M K. Inducible apoptosis as a safety switch for adoptive cell therapy. N Engl J Med. 2011; 365(18):1673-83. Epub 2011/11/04. doi: 10.1056/NEJMoa1106152. PubMed PMID: 22047558; PMCID: PMC3236370.
30. Han X, Wang M, Duan S, Franco P J, Kenty J H, Hedrick P, Xia Y, Allen A, Ferreira L M R, Strominger J L, Melton D A, Meissner T B, Cowan C A. Generation of hypoimmunogenic human pluripotent stem cells. Proc Natl Acad Sci USA. 2019; 116(21):10441-6. Epub 2019/05/02. doi: 10.1073/pnas.1902566116. PubMed PMID: 31040209; PMCID: PMC6535035.
31. Deuse T, Hu X, Gravina A, Wang D, Tediashvili G, De C, Thayer W O, Wahl A, Garcia J V, Reichenspurner H, Davis M M, Lanier L L, Schrepfer S. Hypoimmunogenic derivatives of induced pluripotent stem cells evade immune rejection in fully immunocompetent allogeneic recipients. Nat Biotechnol. 2019; 37(3):252-8. Epub 2019/02/20. doi: 10.1038/s41587-019-0016-3. PubMed PMID: 30778232; PMCID: PMC6419516.

1. A method of producing a mature cardiomyocyte from an immature cardiomyocyte comprising contacting the immature cardiomyocyte with at least one cardiomyocyte maturation factor in three-dimensional culture.

2. (canceled)

3. (canceled)

4. The method of claim 1, wherein the at least one cardiomyocyte maturation factor is selected from the group consisting of nutlin-3a, quercetin, Torin1, and combinations thereof.

5. (canceled)

6. (canceled)

7. (canceled)

8. The method of claim 1, wherein the at least one cardiomyocyte maturation factor is contacted with the immature cardiomyocyte via pulse treatment or continuous treatment.

9. (canceled)

10. The method of claim 1, wherein the immature cardiomyocyte is derived from an iPS cell, an ES cell, a T cell, or a fibroblast.

11. (canceled)

12. (canceled)

13. (canceled)

14. The method of claim 1, wherein the immature cardiomyocyte resembles a fetal cardiomyocyte.

15. The method of claim 1, wherein the immature cardiomyocyte is contacted with the at least one cardiomyocyte maturation factor after the immature cardiomyocyte begins beating.

16. (canceled)

17. The method of claim 1, wherein the mature cardiomyocyte exhibits increased expression of one or more genes of maturation as compared to an immature cardiomyocyte.

18. (canceled)

19. The method of claim 1, wherein the mature cardiomyocyte exhibits increased expression of one or more sarcomeric proteins as compared to an immature cardiomyocyte.

20. (canceled)

21. The method of claim 1, wherein the mature cardiomyocyte exhibits increased expression of one or more ion channel genes as compared to an immature cardiomyocyte.

22. (canceled)

23. The method of claim 1, wherein the mature cardiomyocyte exhibits increased expression of REST and/or GATA4 as compared to an immature cardiomyocyte, exhibits a decreased beating rate as compared to an immature cardiomyocyte, exhibits a resting membrane potential of ≤−70 mV, exhibits a spontaneous beating rate of <40 beats per minute, and/or exhibits an upstroke velocity of >200 V/sec.

24. (canceled)

25. (canceled)

26. (canceled)

27. (canceled)

28. The method of claim 1, wherein the mature cardiomyocyte is an electrically mature cardiomyocyte, is a contractility mature cardiomyocyte, and/or a metabolically mature cardiomyocyte.

29. (canceled)

30. (canceled)

31. (canceled)

32. A non-naturally occurring cardiomyocyte produced by the methods of claim 1.

33. The non-naturally occurring cardiomyocyte of claim 32, wherein the non-naturally occurring cardiomyocyte exhibits increased expression of one or more genes of maturation, one or more sarcomeric proteins, and/or one or more ion channel genes as compared to an immature cardiomyocyte.

34.-39. (canceled)

40. The non-naturally occurring cardiomyocyte of claim 32, wherein the non-naturally occurring cardiomyocyte exhibits a decreased beating rate as compared to an immature cardiomyocyte exhibits a resting membrane potential of ≤−70 mV, exhibits a spontaneous beating rate of <40 beats per minute, and/or exhibits an upstroke velocity of >200 V/sec.

41. (canceled)

42. (canceled)

43. (canceled)

44. The non-naturally occurring cardiomyocyte of claim 32, wherein the non-naturally occurring cardiomyocyte is an electrically mature cardiomyocyte, is a contractility mature cardiomyocyte, and/or a metabolically mature cardiomyocyte.

45. (canceled)

46. (canceled)

47. (canceled)

48. A method of treatment comprising administering to a subject in need thereof a composition comprising a non-naturally occurring cardiomyocyte according to claim 32.

49. The method of claim 48, wherein the subject has, or is at risk of developing, a ventricular arrhythmia, decreased systolic heart function, chronic heart failure, congenital heart disease, or other heart disease.

50. (canceled)

51. (canceled)

52. Use of a composition in the manufacture of a medicament for treatment of a heart condition, wherein the treatment comprises administration of the medicament to a subject in need thereof, wherein the composition comprises at least one non-naturally occurring cardiomyocyte according to claim 32.

53. (canceled)

54. A method of inducing quiescence of a cardiomyocyte comprising contacting a senescent cardiomyocyte with a cardiomyocyte maturation factor in three-dimensional culture, thereby inducing the senescent cardiomyocyte to transition into a quiescent cardiomyocyte.

55.-66. (canceled)

67. A method of producing a mature cardiomyocyte from an immature cardiomyocyte comprising contacting the immature cardiomyocyte with at least one cardiomyocyte maturation factor selected from the group consisting of nutlin-3a, quercetin, Torin1, and combinations thereof.

68.-72. (canceled)
